(19) United States
(12) Patent Application Publication
(10) Pub. No.: US 2016/0029943 A1
(43) Pub. Date: Feb. 4, 2016

(54) INFORMATION ANALYSIS DEVICE, EXERCISE ANALYSIS SYSTEM, INFORMATION ANALYSIS METHOD, ANALYSIS PROGRAM, IMAGE GENERATION DEVICE, IMAGE GENERATION METHOD, IMAGE GENERATION PROGRAM, INFORMATION DISPLAY DEVICE, INFORMATION DISPLAY SYSTEM, INFORMATION DISPLAY PROGRAM, AND INFORMATION DISPLAY METHOD

(71) Applicant: Seiko Epson Corporation, Tokyo (JP)

(72) Inventors: Shunichi MIZUOCHI, Matsumoto-shi (JP); Shuji Uchida, Shiojiri-shi (JP); Ken Watanabe, Matsumoto-shi (JP); Akinobu Sato, Fujimi-machi (JP); Daisuke Sugiya, Chino-shi (JP)

(21) Appl. No.: 14/814,488

(22) Filed: Jul. 30, 2015

(30) Foreign Application Priority Data
Jul. 31, 2014 (JP) 2014-157206
Jul. 31, 2014 (JP) 2014-157209
Jul. 31, 2014 (JP) 2014-157210
Jun. 5, 2015 (JP) 2015-115212

(51) Int. Cl. A61B 5/22; A61B 5/00; A61B 5/103

(52) U.S. Cl. CPC A61B 5/222; A61B 5/1038; A61B 5/0022; A61B 5/7246; A61B 5/7405; A61B 5/7455; A61B 5/743; A61B 5/6823; A61B 5/486; A61B 2562/0219; A61B 2503/10

(57) ABSTRACT

An information analysis device including an exercise analysis information acquisition unit that acquires a plurality of pieces of exercise analysis information that are results of analyzing exercise of a plurality of users, and an analysis information generation unit that generates analysis information from which exercise capabilities of the plurality of users can be compared, using the plurality of pieces of exercise analysis information.

### BACKGROUND

1. Technical Field

The present invention relates to an information analysis device, an exercise analysis system, an information analysis method, an analysis program, an image generation device, an image generation method, an image generation program, an information display device, an information display system, an information display program, and an information display method.

2. Related Art

JP-T-2011-516210 discloses a system capable of measuring exercise data (for example, time, and running distance) of race participants, sorting the measured exercise data according to, for example, age or sex, and performing ranking display. According to this system, each participant can compare his or her result with results of other participants with the same age or sex.

Further, JP-A-2008-289866 describes a system in which a user wears a suit having a number of orientation measurement units embedded therein, and an operation of a person can be tracked with high precision using measurement data of the orientation measurement units. By using information obtained by this system, for example, a three-dimensional image indicating exercise of the user can be expected to be rendered with high accuracy.

Further, in a walking operation or a running operation, it is important to take steps in an appropriate form. A device that indexes exercise so that a user can confirm his or her form has been developed.

For example, JP-T-2013-537436 discloses a device for analyzing biomechanical parameters of a stride of a runner.

However, in the system described in JP-T-2011-516210, each participant can compare a result such as the time or the running distance with that of the other participants, but cannot directly compare exercise capability causing the result of the time or the running distance with that of the other participants. Therefore, the participant (user) cannot obtain valuable information for what to do in order to improve a record and to prevent injury. Further, in the system described in JP-T-2011-516210, the participant (user) can set a target of the time or the running distance of the next race while viewing the time or the running distance of the participant or other participants, but cannot set a target value of each index according to the running capability since no information on various indexes related to the running capability is presented.

Further, in the system described in JP-A-2008-289866, since a large number of orientation measurement units (sensors) are necessary, accurate tracking cannot be performed if relative relationship among positions of all the sensors are not accurately recognized and measurement time of all the sensors is not accurately synchronized. That is, as the number of sensors increases, there is a possibility of tracking motion of various portions of the user more accurately. However, since is difficult to synchronize the sensors, sufficient tracking accuracy is not obtained. Further, for the purpose of evaluating the exercise capability while viewing an image indicating the exercise of the user, states of portions closely related to the exercise capability are desired to be accurately reproduced, but it may not be unnecessary to accurately reproduce states of other portions, and a system requiring a large number of sensors leads to an unnecessary increase in costs.

Also, forms are normally different according to a running environment such as a state of inclination of a running road, or running speed. In JP-T-2013-537436, since there is a possibility of an index of a different form being treated as the same index, there may be a problem in accuracy or usefulness as the index.

### SUMMARY

An advantage of some aspects of the invention is to provide an information analysis device, an exercise analysis system, an information analysis method, and an analysis program capable of presenting information from which exercise capabilities of a plurality of users are comparable. Another advantage of some aspects of the invention is to provide an information analysis device, an exercise analysis system, an information analysis method, and an analysis program that enable a user to appropriately set index values related to exercise capability.

Still another advantage of some aspects of the invention is to provide an image generation device, an exercise analysis system, an image generation method, and an image generation program capable of generating image information for accurately reproducing a running state of the user using information obtained from detection results of a small number of sensors. Yet another advantage of some aspects of the invention is to provide an image generation device, an exercise analysis system, an image generation method, and an image generation program capable of generating image information for accurately reproducing a state of a portion closely related to exercise capability using information obtained from detection results of a small number of sensors.

Still yet another advantage of some aspects of the invention is to provide an information display device, an information display system, an information display program, and an information display method capable of accurately recognizing indexes regarding running of a user can be provided.

The invention can be implemented as the following aspects or application examples.

### APPLICATION EXAMPLE 1

An information analysis device according to this application example includes: an exercise analysis information acquisition unit that acquires a plurality of pieces of exercise analysis information that are results of analyzing exercise of a plurality of users; and an analysis information generation unit that generates analysis information from which exercise capabilities of the plurality of users can be compared, using the plurality of pieces of exercise analysis information.

The exercise capability, for example, may be skill power or may be endurance power.

Each of the plurality of pieces of exercise analysis information may be a result of analyzing the exercise of a plurality of users using a detection result of the inertial sensor. For example, each of the plurality of pieces of exercise analysis information may be generated by one exercise analysis device or may be generated by a plurality of exercise analysis devices.

According to the information analysis device of this application example, it is possible to generate analysis information from which exercise capabilities of the plurality of users are comparable, using the exercise analysis information of the plurality of users, and present the analysis information. Each user can compare the exercise capability of the user with the exercise capabilities of other users using the presented analysis information.

### APPLICATION EXAMPLE 2

In the information analysis device according to the application example, the analysis information generation unit may generate the analysis information from which exercise capabilities of the plurality of users are comparable each time the plurality of users perform the exercise.

Each time the exercise is performed may be, for example, daily, monthly, or a unit determined by the user.

According to the exercise analysis device of this application example, each user can recognize a transition of a difference in exercise capability with another user from presented analysis information.

### APPLICATION EXAMPLE 3

In the information analysis device according to the application example, the plurality of users may be classified into a plurality of groups, and the analysis information generation unit may generate the analysis information from which exercise capabilities of the plurality of users are comparable for each group.

According to the exercise analysis device of this application example, each user can compare exercise capability of the user with exercise capability of another user belonging to the same group as the user using the presented analysis information.

### APPLICATION EXAMPLE 4

In the information analysis device according to the application example, each of the plurality of pieces of exercise analysis information may include a value of an index regarding exercise capability of each of the plurality of users, and the analysis information generation unit may generate the analysis information from which exercise capability of the first user included in the plurality of users is relatively evaluable, using the values of the indexes of the plurality of users.

According to the information analysis device according to this application example, the first user can relatively evaluate exercise capability of the first user among the plurality of users using the presented analysis information.

### APPLICATION EXAMPLE 5

In the information analysis device according to the application example, each of the plurality of pieces of exercise analysis information may include a value of an index regarding exercise capability of each the plurality of users, the information analysis device may include a target value acquisition unit that acquires a target value of the index of the first user included in the plurality of users, and the analysis information generation unit may generate the analysis information from which the value of the index of the first user is comparable with the target value.

According to the exercise analysis device of this application example, the first user can appropriately set the target value for each index according to the exercise capability of the user while viewing the analysis information presented by the information analysis device. The first user can recognize a difference between the exercise capability of the user and the target value using the presented analysis information.

### APPLICATION EXAMPLE 6

In the information analysis device according to the application example, the index may be at least one of ground time, stride, energy, a directly-under landing rate, propulsion efficiency, a flow of a leg, an amount of brake at the time of landing, and landing shock.

### APPLICATION EXAMPLE 7

In the information analysis device according to the application example, the exercise capability may be skill power or endurance power.

### APPLICATION EXAMPLE 8

An exercise analysis system according to this application example includes: an exercise analysis device that analyzes exercise of a user using a detection result of an inertial sensor and generates exercise analysis information that is information on an analysis result; and the information analysis device according to any of the application examples described above.

According to the exercise analysis system of this application example, analysis information from which exercise capabilities of the plurality of users can be compared can be generated using a plurality of pieces of exercise analysis information as a result of accurately analyzing the exercises of the plurality of users using the detection result of the inertial sensor, and presented. Thus, each user can compare exercise capability of the user with exercise capability of another user using the presented analysis information.

### APPLICATION EXAMPLE 9

In the exercise analysis system according to the application example, the information analysis device may further including: a reporting device that reports information on an exercise state during exercise of a first user included in the plurality of users, the information analysis device transmits the target value to the reporting device, the exercise analysis device transmits a value of the index to the reporting device during exercise of the first user, and the reporting device receives the target value and the value of the index, compares the value of the index with the target value, and reports information on the exercise state according to a comparison result.

According to the exercise analysis system of this application example, the first user can exercise while recognizing the difference between the index value during exercise and an appropriate target value based on the analysis information of past exercise.

### APPLICATION EXAMPLE 10

In the information analysis device according to the application example, the reporting device may report information on the exercise state through sound or vibration.

Reporting through sound or vibration has small influence on the exercise state, and thus, according to the exercise analysis system of this application example, the first user can recognize the exercise state without obstruction of the exercise.

### APPLICATION EXAMPLE 11

An information analysis method according to this application example includes: acquiring a plurality of pieces of exercise analysis information that is a result of analyzing exercises of a plurality of users using a detection result of an inertial sensor; and generating analysis information from which exercise capabilities of the plurality of users can be compared, using the plurality of pieces of exercise analysis information.

According to the exercise analysis method of this application example, analysis information from which exercise capabilities of the plurality of users can be compared can be generated using a plurality of pieces of exercise analysis information as a result of accurately analyzing the exercises of the plurality of users using the detection result of the inertial sensor, and presented. Thus, each user can compare exercise capability of the user with exercise capability of another user using the presented analysis information.

### APPLICATION EXAMPLE 12

An analysis program according to this application example causes a computer to execute: acquisition of a plurality of pieces of exercise analysis information that is a result of analyzing exercises of a plurality of users using a detection result of an inertial sensor; and generation of analysis information from which exercise capabilities of the plurality of users can be compared, using the plurality of pieces of exercise analysis information.

According to the analysis program of this application example, analysis information from which exercise capabilities of the plurality of users can be compared can be generated using a plurality of pieces of exercise analysis information as a result of accurately analyzing the exercises of the plurality of users using the detection result of the inertial sensor, and presented. Thus, each user can compare exercise capability of the user with exercise capability of another user using the presented analysis information.

### APPLICATION EXAMPLE 13

An image generation device according to this application example includes: an exercise analysis information acquisition unit that acquires exercise analysis information of a user at the time of running, the exercise analysis information being generated using a detection result of an inertial sensor; and an image information generation unit that generates image information in which the exercise analysis information is associated with image data of a user object indicating running of the user.

Since an inertial sensor can detect a fine motion of a portion of a user wearing the inertial sensor, it is possible to accurately generate the exercise analysis information of the user at the time of running using detection results of a small number (for example, one) of inertial sensors. Therefore, according to the image generation device of this application example, it is possible to generate, for example, image information for accurately reproducing a running state of the user using the exercise analysis information of the user obtained from the detection results of the small number of sensors.

### APPLICATION EXAMPLE 14

In the image generation device according to the application example, the exercise analysis information may include a value of at least one index regarding exercise capability of the user.

The exercise capability, for example, may be skill power or may be endurance power.

### APPLICATION EXAMPLE 15

In the image generation device according to the application example, the image information generation unit may calculate a value of at least one index regarding exercise capability of the user using the exercise analysis information.

According to the image generation device of this application example, it is possible to generate, for example, image information for accurately reproducing a state of a portion closely related to exercise capability of the user using a value of at least one index regarding the exercise capability of the user. Therefore, the user can visually clearly recognize, for example, a state of a most desired portion using the image information although the user does not recognize a motion of the entire body.

### APPLICATION EXAMPLE 16

In the image generation device according to the application example, the exercise analysis information may include information on the posture angle of the user, and the image information generation unit may generate the image information using the value of the index and the information on the posture angle.

According to the exercise analysis device of this application example, it is possible to generate image information for accurately reproducing states of more portions using the information on the posture angle.

### APPLICATION EXAMPLE 17

In the image generation device according to the application example, the image information generation unit may generate comparison image data for comparison with the image data, and generates the image information including the image data and the comparison image data.

According to the image generation device according to this application example, a user can easily compare an exercise state of the user with an exercise state of a comparison target and objectively evaluate exercise capability of the user.

### APPLICATION EXAMPLE 18

In the image generation device according to the application example, the image data may be image data indicating an exercise state at a feature point of the exercise of the user.

Information on the feature point of the exercise of the user may be included in the exercise analysis information, and the image information generation unit may detect the feature point of the exercise of the user using the exercise analysis information.

According to the image generation device of this application example, it is possible to generate image information for accurately reproducing a state of a portion that is closely related to exercise capability at a feature point that is particularly important to evaluation of exercise capability.

### APPLICATION EXAMPLE 19

In the image generation device according to the application example, the feature point may be a time at when a foot of the user lands, a time of mid-stance, or a time of kicking.

According to the image generation device of this application example, it is possible to generate image information for accurately reproducing a state of a portion that is closely related to exercise capability or the like at a timing of landing, mid-stance, and kicking that are particularly important to evaluation of running capability.

### APPLICATION EXAMPLE 20

In the image generation device according to the application example, the image information generation unit may generate the image information including a plurality of pieces of image data respectively indicating exercise states at multiple types of feature points of the exercise of the user.

According to the image generation device of this application example, it is possible to generate image information for accurately reproducing a state of a portion that is closely related to exercise capability at multiple types of feature points that are particularly important to evaluation of exercise capability.

### APPLICATION EXAMPLE 21

In the image generation device according to the application example, at least one of multiple types of feature points may be a time at when a foot of the user lands, a time of mid-stance, or a time of kicking.

### APPLICATION EXAMPLE 22

In the image generation device according to the application example, in the image information, the plurality of pieces of image data may be arranged side by side on a time axis or a space axis.

According to the image generation device of this application example, it is possible to generate image information for reproducing a relationship of a time or a position between a plurality of states at multiple types of feature points of a portion closely related to the exercise capability.

### APPLICATION EXAMPLE 23

In the image generation device according to the application example, the image information generation unit may generate a plurality of pieces of supplement image data for supplementing the plurality of pieces of image data on a time axis or on a spatial axis, and may generate the image information including moving image data having the plurality of pieces of image data and the plurality of pieces of supplement image data.

According to the image generation device according to this application example, it is possible to generate image information for accurately reproducing a continuous motion of a portion closely related to exercise capability.

### APPLICATION EXAMPLE 24

In the image generation device according to the application example, the inertial sensor may be mounted to a torso of the user.

According to the image generation device according to this application example, it is possible to generate image information for accurately reproducing a state of a torso closely related to exercise capability in multiple types of exercises using the information obtained from the detection result of one inertial sensor. Further, it is possible to also estimate a state of another portion, such as a leg or an arm from the state of the torso, and thus, according to the image generation device of the application example, it is possible to generate image information for accurately reproducing the state of multiple portions using the information obtained from the detection result of one inertial sensors.

### APPLICATION EXAMPLE 25

An exercise analysis system according to this application example includes: the image generation device according to any of the application examples described above; and an exercise analysis device that generates the exercise analysis information.

### APPLICATION EXAMPLE 26

An image generation method according to this application example includes: acquiring exercise analysis information of a user at the time of running, the exercise analysis information being generated using a detection result of an inertial sensor; and generating image information in which the exercise analysis information is associated with image data of a user object indicating running of the user.

According to the image generation method of this application example, it is possible to generate, for example, image information for accurately reproducing a running state of the user using exercise analysis information that is accurately generated using a detection result of an inertial sensor capable of detecting a fine motion of a user.

### APPLICATION EXAMPLE 27

An image generation program according to this application example causes a computer to execute: acquisition of exercise analysis information of a user at the time of running, the exercise analysis information being generated using a detection result of an inertial sensor; and generation of image information in which the exercise analysis information is associated with image data of a user object indicating running of the user.

According to the image generation program of this application example, it is possible to generate, for example, image information for accurately reproducing a running state of the user using exercise analysis information that is accurately generated using a detection result of an inertial sensor capable of detecting a fine motion of a user.

### APPLICATION EXAMPLE 28

An information display device according to this application example includes: a display unit that displays running state information that is information on at least one of running speed and a running environment of a user, and an index regarding running of the user calculated using a detection result of an inertial sensors in association with each other.

According to the information display device of this application example, since running speed or a running environment that easily affects the form, and indexes regarding running of the user are displayed in association with each other, indexes of different forms primarily caused by a difference in the running state can be divided and displayed. Therefore, it is possible to implement an information display device capable of accurately recognizing indexes regarding the running of the user.

### APPLICATION EXAMPLE 29

In the information display device according to the application example, the running environment may be a state of a slope of a running road.

According to the information display device of this application example, indexes of different forms primarily caused by a difference in the running state can be divided and displayed by adopting a state of a slope of a running road that easily affects the form, as a running state. Therefore, it is possible to implement an information display system capable of accurately recognizing indexes regarding the running of the user.

### APPLICATION EXAMPLE 30

In the information display device according to the application example, the index may be any one of directly-under landing, propulsion efficiency, a flow of a leg, a running pitch, and landing shock.

According to the information display device of this application example, it is possible to provide information useful for improvement of the exercise to the user.

### APPLICATION EXAMPLE 31

An information display system according to this application example includes: a calculation unit that calculates an index regarding running of a user using a detection result of an inertial sensor; and a display unit that displays running state information that is information on at least one of running speed and a running environment of the user, and the index in association with each other.

According to the information display system of this application example, since running speed or a running environment that easily affects the form, and indexes regarding running of the user are displayed in association with each other, indexes of different forms primarily caused by a difference in the running state can be divided and displayed. Therefore, it is possible to implement an information display system capable of accurately recognizing indexes regarding the running of the user.

### APPLICATION EXAMPLE 32

In the information display system according to the application example, the information display system may further include a determination unit that measures at least one of the running speed and the running environment.

According to this application example, since the measurement unit measures at least one of the running speed and the running environment of the user, it is possible to implement an information display system capable of reducing input manipulations of the user.

### APPLICATION EXAMPLE 33

An information display program according to this application example causes a computer to execute: displaying of running state information that is information on at least one of running speed and a running environment of a user, and an index regarding running of the user calculated using a detection result of an inertial sensors in association with each other.

According to the information display program of this application example, since running speed or a running environment that easily affects the form, and indexes regarding running of the user are displayed in association with each other, indexes of different forms primarily caused by a difference in the running state can be divided and displayed. Therefore, it is possible to implement an information display program capable of accurately recognizing indexes regarding the running of the user.

### APPLICATION EXAMPLE 34

An information display method according to this application example includes: displaying running state information that is information on at least one of running speed and a running environment of a user, and an index regarding running of the user calculated using a detection result of an inertial sensors in association with each other.

According to the information display method of this application example, since running speed or a running environment that easily affects the form, and indexes regarding running of the user are displayed in association with each other, indexes of different forms primarily caused by a difference in the running state can be divided and displayed. Therefore, it is possible to implement an information display method capable of accurately recognizing indexes regarding the running of the user.

### BRIEF DESCRIPTION OF THE DRAWINGS

The invention will be described with reference to the accompanying drawings, wherein like numbers reference like elements.

FIG. 1 is a diagram illustrating an example of a configuration of an exercise analysis system of a first embodiment.

FIG. 2 is an illustrative diagram of an overview of the exercise analysis system of the first embodiment.

FIG. 3 is a functional block diagram illustrating an example of a configuration of an exercise analysis device in the first embodiment.

FIG. 4 is a diagram illustrating an example of a configuration of a sensing data table.

FIG. 5 is a diagram illustrating an example of a configuration of a GPS data table.

FIG. 6 is a diagram illustrating an example of a configuration of a geomagnetic data table.

FIG. 7 is a diagram illustrating an example of a configuration of an operation data table.

FIG. 8 is a functional block diagram illustrating an example of a configuration of a processing unit of the exercise analysis device of the first embodiment.

FIG. 9 is a functional block diagram illustrating an example of a configuration of an inertial navigation operation unit.

FIG. 10 is an illustrative diagram of a posture at the time of running of a user.

FIG. 11 is an illustrative diagram of a yaw angle at the time of running of the user.

FIG. 12 is a diagram illustrating an example of 3-axis acceleration at the time of running of the user.

FIG. 13 is a functional block diagram illustrating an example of a configuration of the exercise analysis device in the first embodiment.

FIG. 14 is a flowchart diagram illustrating an example of a procedure of an exercise analysis process.

FIG. 15 is a flowchart diagram illustrating an example of a procedure of an inertial navigation operation process.

FIG. 16 is a flowchart diagram illustrating an example of a procedure of a running detection process.

FIG. 17 is a flowchart diagram illustrating an example of a procedure of an exercise analysis information generation process in the first embodiment.

FIG. 18 is a functional block diagram illustrating an example of a configuration of a reporting device.

FIGS. 19A and 19B are diagrams illustrating examples of information displayed on a display unit of the reporting device.

FIG. 20 is a flowchart diagram illustrating an example of a procedure of a reporting process in the first embodiment.

FIG. 21 is a functional block diagram illustrating an example of a configuration of the information analysis device.

FIG. 22 is a flowchart diagram illustrating an example of a procedure of an analysis process.

FIG. 23 is a diagram illustrating an example of a screen displayed on a display unit of the information analysis device.

FIG. 24 is a diagram illustrating an example of a screen displayed on a display unit of the information analysis device.

FIG. 25 is a diagram illustrating an example of a screen displayed on a display unit of the information analysis device.

FIG. 26 is a diagram illustrating an example of a screen displayed on a display unit of the information analysis device.

FIG. 27 is a diagram illustrating an example of a screen displayed on a display unit of the information analysis device.

FIG. 28 is a diagram illustrating an example of a screen displayed on a display unit of the information analysis device.

FIG. 29 is a diagram illustrating an example of a screen displayed on a display unit of the information analysis device.

FIG. 30 is a diagram illustrating an example of a screen displayed on a display unit of the information analysis device.

FIG. 31 is a diagram illustrating an example of a screen displayed on a display unit of the information analysis device.

FIG. 32 is a diagram illustrating an example of a screen displayed on a display unit of the information analysis device.

FIG. 33 is a diagram illustrating an example of a screen displayed on a display unit of the information analysis device.

FIG. 34 is a diagram illustrating an example of a configuration of an exercise analysis system of a second embodiment.

FIG. 35 is a functional block diagram illustrating an example of a configuration of an image generation device.

FIGS. 36A to 36C are diagrams illustrating examples of image data (user object) at the time of landing.

FIGS. 37A to 37C are diagrams illustrating examples of comparison image data (comparison object) at the time of landing.

FIGS. 38A to 38C are diagrams illustrating examples of image data (user object) at the time of mid-stance.

FIGS. 39A to 39C are diagrams illustrating examples of comparison image data (comparison object) of the mid-stance.

FIGS. 40A to 40C are diagrams illustrating examples of image data (user object) at the time of kicking.

FIGS. 41A to 41C are diagrams illustrating examples of comparison image data (comparison user object) at the time of kicking.

FIG. 42 is a diagram illustrating an example of an image displayed on the display unit of the image generating device.

FIG. 43 is a diagram illustrating another example of an image displayed on the display unit of the image generating device.

FIG. 44 is a diagram illustrating another example of an image displayed on the display unit of the image generating device.

FIG. 45 is a flowchart diagram illustrating an example of a procedure of the image generation process.

FIG. 46 is a flowchart diagram illustrating an example of a procedure of an image generation and display process of mode 1.

FIG. 47 is a flowchart diagram illustrating an example of a procedure of an image generation and display process of mode 2.

FIG. 48 is a flowchart diagram illustrating an example of a procedure of an image generation and display process of mode 3.

FIG. 49 is a flowchart diagram illustrating an example of a procedure of an image generation and display process of mode 4.

FIG. 50 is a flowchart diagram illustrating an example of a procedure of an image data generation process at the time of landing.

FIG. 51 is a flowchart diagram illustrating an example of a procedure of an image data generation process at the time of mid-stance.

FIG. 52 is a flowchart diagram illustrating an example of a procedure of an image data generation process at the time of kicking.

FIG. 53 illustrates an example of a configuration of an information display system according to a third embodiment.

FIG. 54 is a functional block diagram illustrating an example of a configuration of an exercise analysis system of the third embodiment.

FIG. 55 is a functional block diagram illustrating an example of a configuration of a processing unit of the exercise analysis system in the third embodiment.

FIG. 56 is a functional block diagram illustrating an example of a configuration of the exercise analysis device in the third embodiment.

FIG. 57 is a diagram illustrating an example of a configuration of a data table of running result information and exercise analysis information.

FIG. 58 is a flowchart diagram illustrating an example of a procedure of an exercise analysis information generation process in the third embodiment.

FIG. 59 is a functional block diagram illustrating an example of a configuration of the reporting device.

FIG. 60 is a flowchart diagram illustrating an example of a procedure of a reporting process in the third embodiment.

FIG. 61 is a functional block diagram illustrating an example of a configuration of the information display device.

FIG. 62 is a flowchart diagram illustrating an example of a procedure of a display process performed by a processing unit of the information display device.

FIG. 63 is a diagram illustrating an example of exercise analysis information displayed on a display unit of the information display device.

### DESCRIPTION OF EXEMPLARY EMBODIMENTS

An exercise analysis system of the present embodiment includes an exercise analysis device that analyzes exercise of the user using a detection result of an inertial sensor and generates exercise analysis information that is information on an analysis result, and information analysis device, and the information analysis device includes an exercise analysis information acquisition unit that acquires a plurality of pieces of exercise analysis information that are results of analyzing exercise of a plurality of users, and an analysis information generation unit that generates analysis information from which exercise capabilities of the plurality of users can be compared, using the plurality of pieces of exercise analysis information.

The exercise capability, for example, may be skill power or may be endurance power.

Each of the plurality of pieces of exercise analysis information may be generated by one exercise analysis device or may be generated by a plurality of exercise analysis devices.

According to the exercise analysis system of the present embodiment, since the inertial sensor can also detect a fine motion of the user, the exercise analysis device can accurately analyze the exercise of the user using a detection result of the inertial sensor. Therefore, according to the exercise analysis system of the present embodiment, the information analysis device can generate analysis information from which exercise capabilities of the plurality of users are comparable, using the exercise analysis information of the plurality of users, and present the analysis information. Each user can compare the exercise capability of the user with the exercise capabilities of other users using the presented analysis information.

In the exercise analysis system of the present embodiment, the analysis information generation unit may generate the analysis information from which exercise capabilities of the plurality of users are comparable each time the plurality of users perform the exercise.

Each time the exercise is performed may be, for example, daily, monthly, or a unit determined by the user.

According to the exercise analysis system of the present embodiment, each user can recognize a transition of a difference in exercise capability with another user from presented analysis information.

In the exercise analysis system of the present embodiment, the plurality of users may be classified into a plurality of groups, and the analysis information generation unit may generate the analysis information from which exercise capabilities of the plurality of users are comparable for each group.

According to the exercise analysis system of the present embodiment, each user can compare exercise capability of the user with exercise capability of another user belonging to the same group as the user using the presented analysis information.

In the exercise analysis system of the present embodiment, each of the plurality of pieces of exercise analysis information may include a value of the index regarding exercise capability of each of the plurality of users, and the analysis information generation unit may generate the analysis information from which exercise capability of the first user included in the plurality of users is relatively evaluable, using the values of the indexes of the plurality of users.

According to the exercise analysis system of the present embodiment, the first user can relatively evaluate exercise capability of the first user among the plurality of users using the presented analysis information.

In the exercise analysis system of the present embodiment, each of the plurality of pieces of exercise analysis information may include a value of an index regarding exercise capability of each the plurality of users, the information analysis device may include a target value acquisition unit that acquires a target value of an index of a first user included in the plurality of users, and the analysis information generation unit may generate the analysis information from which the value of the index of the first user is comparable with the target value.

According to the exercise analysis system of the present embodiment, the first user can appropriately set the target value for each index according to the exercise capability of the user while viewing the analysis information presented by the information analysis device. The first user can recognize a difference between the exercise capability of the user and the target value using the presented analysis information.

The exercise analysis system of the present embodiment may include a reporting device that reports the information on the exercise state during the exercise of the first user, the information analysis device may transmit the target value to the reporting device, the exercise analysis device may transmit a value of the index to the reporting device during exercise of the first user, and the reporting device may receive the target value and the value of the index, compare the value of the index with the target value, and report information on the exercise state according to a comparison result.

According to the exercise analysis system of the present embodiment, the first user can exercise while recognizing the difference between the index value during exercise and an appropriate target value based on the analysis information of past exercise.

In the exercise analysis system of the present embodiment, the reporting device may report information on the exercise state through sound or vibration.

Reporting through sound or vibration has small influence on the exercise state, and thus, according to the exercise analysis system of the present embodiment, the first user can recognize the exercise state without obstruction of the exercise.

In the exercise analysis system of the present embodiment, the exercise capability may be skill power or endurance power.

In the exercise analysis system of the present embodiment, the index may be at least one of ground time, stride, energy, a directly-under landing rate, propulsion efficiency, a flow of a leg, an amount of brake at the time of landing, and landing shock.

The information analysis device of the present embodiment includes an exercise analysis information acquisition unit that acquires a plurality of pieces of exercise analysis information that are results of analyzing exercise of a plurality of users using the detection result of the inertial sensor, and an analysis information generation unit that generates analysis information from which exercise capabilities of the plurality of users can be compared, using the plurality of pieces of exercise analysis information.

According to the information analysis device of the present embodiment, analysis information from which exercise capabilities of the plurality of users can be compared can be generated using a plurality of pieces of exercise analysis information as a result of accurately analyzing the exercises of the plurality of users using the detection result of the inertial sensor, and presented. Thus, each user can compare exercise capability of the user with exercise capability of another user using the presented analysis information.

An information analysis method of the present embodiment includes acquiring a plurality of pieces of exercise analysis information as a result of analyzing the exercises of the plurality of users using the detection result of the inertial sensor, and generating analysis information from which exercise capabilities of the plurality of users can be compared.

According to the information analysis method of the present embodiment, analysis information from which exercise capabilities of the plurality of users can be compared can be generated using a plurality of pieces of exercise analysis information as a result of accurately analyzing the exercises of the plurality of users using the detection result of the inertial sensor, and presented. Thus, each user can compare exercise capability of the user with exercise capability of another user using the presented analysis information.

A program of the present embodiment causes a computer to implement acquisition of a plurality of pieces of exercise analysis information as a result of analyzing the exercises of the plurality of users using the detection result of the inertial sensor, and generation of analysis information from which exercise capabilities of the plurality of users can be compared using the plurality of pieces of exercise analysis information.

According to the program of the present embodiment, analysis information from which exercise capabilities of the plurality of users can be compared can be generated using the plurality of pieces of exercise analysis information as a result of accurately analyzing the exercises of the plurality of users using the detection result of the inertial sensor, and presented. Thus, each user can compare exercise capability of the user with exercise capability of another user using the presented analysis information.

The image generation device of the present embodiment includes an image information generation unit that generates image information including image data indicating an exercise state of the user using the value of the index regarding the exercise capability of the user obtained by analyzing the exercise of the user using the detection result of the inertial sensor.

The exercise capability, for example, may be skill power or may be endurance power.

Since an inertial sensor can detect a fine motion of a portion of a user wearing the inertial sensor, it is possible to accurately calculate a value of an index regarding exercise capability of the user using detection results of a small number (for example, one) of inertial sensors. Therefore, according to the image generation device of the present embodiment, it is possible to generate image information for accurately reproducing a state of a portion closely related to exercise capability using the value of the index related to the exercise capability of the user obtained from the detection results of the small number of sensors. Therefore, the user can visually clearly recognize a state of a most desired portion using the image information although the user does not recognize a motion of the entire body.

The image generation device of the present embodiment includes an exercise analysis information acquisition unit that acquires exercise analysis information that is information on a result of analyzing the exercise of the user using the detection result of the inertial sensor, and the image information generation unit may generate the image information using the exercise analysis information.

In the image generation device of the present embodiment, the exercise analysis information may include a value of at least one index.

In the image generation device of the present embodiment, the image information generation unit may calculate a value of at least one index using the exercise analysis information.

In the image generation device of the present embodiment, the exercise analysis information may include information on the posture angle of the user, and the image information generation unit may generate the image information using the value of the index and the information on the posture angle.

According to the exercise analysis device of the present embodiment, it is possible to generate image information for accurately reproducing states of more portions using the information on the posture angle.

In the image generation device of the present embodiment, the image information generation unit may generate comparison image data for comparison with the image data and generate the image information including the image data and the comparison image data.

According to the image generation device of the present embodiment, the user can easily compare an exercise state of the user with an exercise state of a comparison target and objectively evaluate exercise capability of the user.

In the image generation device of the present embodiment, the image data may be image data indicating an exercise state at a feature point of the exercise of the user.

Information on the feature point of the exercise of the user may be included in the exercise analysis information, and the image information generation unit may detect the feature point of the exercise of the user using the exercise analysis information.

According to the image generation device of the present embodiment, it is possible to generate image information for accurately reproducing a state of a portion that is closely related to exercise capability at a feature point that is particularly important to evaluation of exercise capability.

In the image generation device of the present embodiment, the feature point may be a time when the foot of the user lands, a time of mid-stance, or a time when the user kicks.

According to the image generation device of the present embodiment, it is possible to generate image information for accurately reproducing a state of a portion that is closely related to exercise capability or the like at a timing of landing, mid-stance, and kicking that are particularly important to evaluation of running capability.

In the image generation device of the present embodiment, the image information generation unit may generate the image information including a plurality of pieces of image data indicating exercise states at multiple types of feature points of the exercise of the user.

According to the image generation device of the present embodiment, it is possible to generate image information for accurately reproducing a state of a portion that is closely related to exercise capability at multiple types of feature points that are particularly important to evaluation of exercise capability.

In the image generation device of the present embodiment, at least one of the multiple types of feature points may be a time when the foot of the user lands, a time of mid-stance, or a time when the user kicks.

In the image generation device of the present embodiment, in the image information, the plurality of pieces of image data may be arranged side by side on a time axis or a space axis.

According to the image generation device of the present embodiment, it is possible to generate image information for reproducing a relationship of a time or a position between a plurality of states at multiple types of feature points of a portion closely related to the exercise capability.

In the image generation device of the present embodiment, the image information generation unit may generate a plurality of pieces of supplement image data for supplementing the plurality of pieces of image data on a time axis or on a spatial axis, and may generate the image information including image data having the plurality of pieces of image data and the plurality of pieces of supplement image data.

According to the image generation device of the present embodiment, it is possible to generate image information for accurately reproducing a continuous motion of a portion closely related to exercise capability.

In the image generation device of the present embodiment, the inertial sensor may be mounted on a torso of the user.

According to the image generation device of the present embodiment, it is possible to generate image information for accurately reproducing a state of a torso closely related to exercise capability in multiple types of exercises using the information obtained from the detection result of one inertial sensor. Further, it is possible to also estimate a state of another portion, such as a leg or an arm from the state of the torso, and thus, according to the image generation device of the present embodiment, it is possible to generate image information for accurately reproducing the state of multiple portions using the information obtained from the detection result of one inertial sensors.

The motion analysis system of the present embodiment includes any one of the image generation devices described above, and an exercise analysis device that calculates the value of the index.

The image generation method of the present embodiment includes generating image information including image data indicating an exercise state of the user using the value of the index regarding the exercise capability of the user obtained by analyzing the exercise of the user using the detection result of the inertial sensor.

According to the image generation method of the present embodiment, it is possible to generate image information for accurately reproducing a state of a portion closely related to the exercise capability using the value of the index related to exercise capability accurately calculated using the detection result of the inertial sensor capable of detecting fine motion of the user.

The program of the present embodiment causes a computer to execute generation of image information including image data indicating an exercise state of the user using the value of the index regarding the exercise capability of the user obtained by analyzing the exercise of the user using the detection result of the inertial sensor.

According to the program of the present embodiment, it is possible to generate image information for accurately reproducing a state of a portion closely related to the exercise capability using the value of the index related to exercise capability accurately calculated using the detection result of the inertial sensor capable of detecting fine motion of the user.

The information display system of the present embodiment is an information display system including a calculation unit that calculates an index regarding exercise of the user based on the output of the inertial sensor mounted on the user, and a display unit that displays running state information that is information on a running state of the user, and the index in association with each other.

According to the information display system of the present embodiment, since the running state information and the index are displayed in association with each other, indexes of different forms primarily caused by a difference in the running state can be divided and displayed. Therefore, it is possible to implement an information display system capable of accurately recognizing indexes regarding the exercise of the user.

The information display system of the embodiment may further include a determination unit that measures the running state.

According to the information display system of the present embodiment, since the determination unit measures the running state, it is possible to implement an information display system capable of reducing input manipulations of the user.

In the information display system according to the embodiment, the running state may be at least one of running speed and running environment.

In the information display system according to the embodiment, the running environment may be a state of inclination of a running road.

According to the information display system of the embodiment, indexes of different forms primarily caused by a difference in the running state can be divided and displayed by adopting the running speed or a state of a slope of a running road that easily affects a form, as a running state. Therefore, it is possible to implement an information display system capable of accurately recognizing indexes regarding the exercise of the user.

In the information display system of the embodiment, the index may be any one of directly-under landing, propulsion efficiency, a flow of a leg, a running pitch, and landing shock.

According to the information display system of the embodiment, it is possible to provide the user with information useful for improving the exercise.

The information display device of the present embodiment is an information display device including a calculation unit that calculates an index regarding exercise of the user based on the output of the inertial sensor mounted on the user, and a display unit that displays running state information that is information on a running state of the user, and the index in association with each other.

According to the information display device of the present embodiment, since the running state information and the index are displayed in association with each other, indexes of different forms primarily caused by a difference in the running state can be divided and displayed. Therefore, it is possible to implement an information display device capable of accurately recognizing indexes regarding the exercise of the user.

An information display program of the present embodiment is an information display program that causes a computer to function as a calculation unit that calculates an index regarding exercise of the user based on the output of the inertial sensor mounted on the user, and a display unit that displays running state information that is information on a running state of the user, and the index in association with each other.

According to the information display program of the present embodiment, since the running state information and the index are displayed in association with each other, indexes of different forms primarily caused by a difference in the running state can be divided and displayed. Therefore, it is possible to implement an information display program capable of accurately recognizing indexes regarding the exercise of the user.

The information display method of the present embodiment is an information display method including a calculation step of calculating an index regarding exercise of the user based on the output of the inertial sensor mounted on the user, a display step of displaying running state information that is information on the running state of the user, and the index in association with each other.

According to the information display method of the present embodiment, since the running state information and the index are displayed in association with each other, indexes of different forms primarily caused by a difference in the running state can be divided and displayed. Therefore, it is possible to implement an information display method capable of accurately recognizing indexes regarding the exercise of the user.

Hereinafter, preferred embodiments of the invention will be described in detail with reference to the accompanying drawings. Also, the embodiments described hereinafter do not unfairly limit the content of the invention described in the appended claims. Further, not all of configurations described hereinafter are essential configuration requirements of the invention.

### 1. First Embodiment

### 1-1. Configuration of Exercise Analysis System

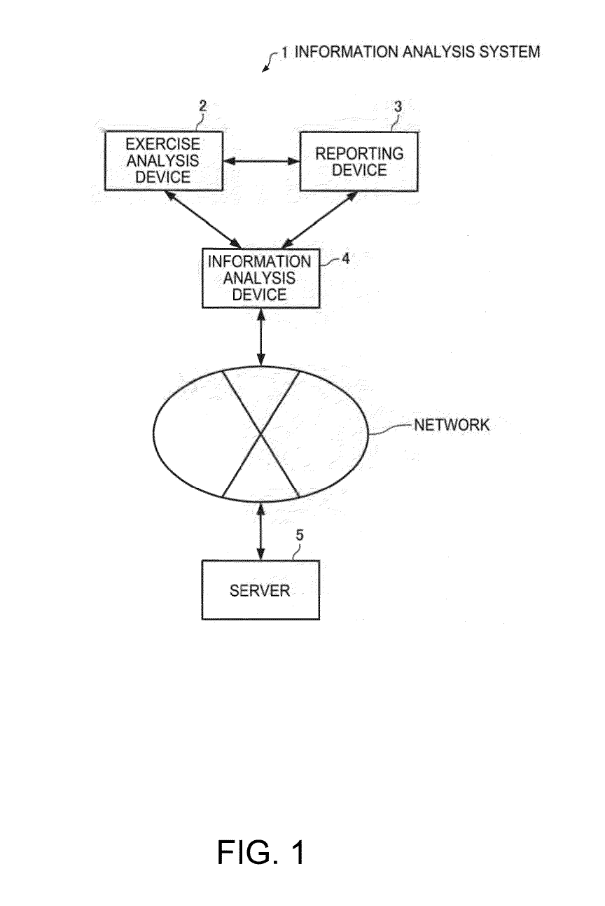

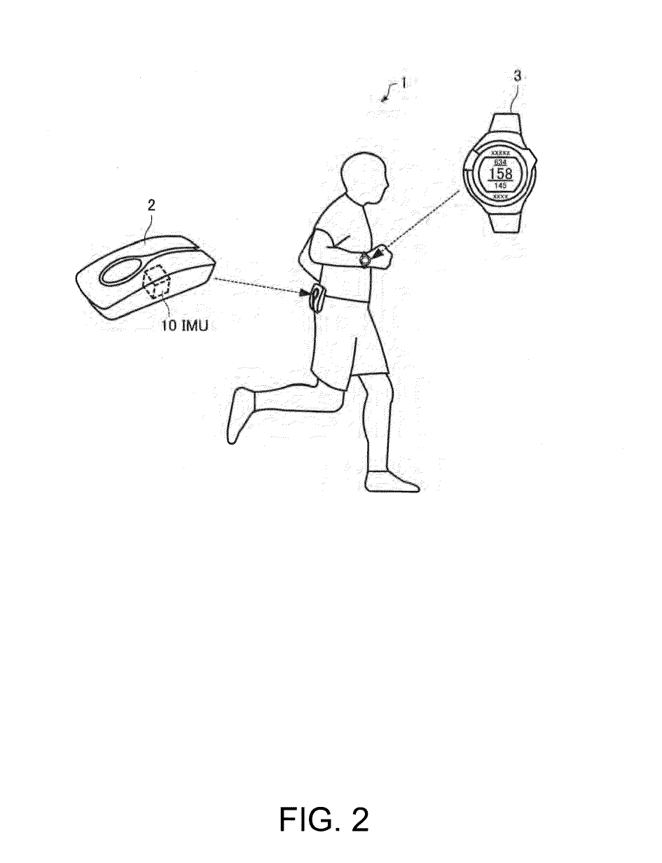

Hereinafter, an exercise analysis system that analyzes exercise in running (including walking) of a user will be described by way of example, but an exercise analysis system of a first embodiment may be an exercise analysis system that analyzes exercise other than running FIG. 1 is a diagram illustrating an example of a configuration of an exercise analysis system 1 of the first embodiment. As illustrated in FIG. 1, the exercise analysis system 1 of the first embodiment includes an exercise analysis device 2, a reporting device 3, and an information analysis device 4. The motion analysis device 2 is a device that analyzes exercise during running of the user, and the reporting device 3 is a device that notifies the user of information on a state during running of the user or a running result. The information analysis device 4 is a device that analyzes and presents a running result after running of the user ends. In the present embodiment, as illustrated in FIG. 2, the exercise analysis device 2 includes an inertial measurement unit (IMU) 10, and is mounted to a torso portion (for example, a right waist, a left waist, or a central portion of waist) of the user so that one detection axis (hereinafter referred to as a z-axis) of the inertial measurement unit (IMU) 10 substantially matches a gravitational acceleration direction (vertically downward) in a state in which the user is at rest. Further, the reporting device 3 is a wrist type (wristwatch type) portable information device, and is mounted on, for example, the wrist of the user. However, the reporting device 3 may be a portable information device, such as a head mount display (HMD) or a smartphone.

The user operates the reporting device 3 at the time of running start to instruct the exercise analysis device 2 to start measurement (inertial navigation operation process and exercise analysis process to be described below), and operates the reporting device 3 at the time of running end to instruct to end the measurement in the exercise analysis device 2. The reporting device 3 transmits a command for instructing start or end of the measurement to the exercise analysis device 2 in response to the operation of the user.

When the exercise analysis device 2 receives the measurement start command, the exercise analysis device 2 starts the measurement using an inertial measurement unit (IMU) 10, calculates values for various exercise indexes which are indexes regarding running capability (an example of exercise capability) of the user using a measurement result, and generates exercise analysis information including the values of the various exercise indexes as information on the analysis result of the running operation of the user. The exercise analysis device 2 generates information to be output during running of the user (output information during running) using the generated exercise analysis information, and transmits the information to the reporting device 3. The reporting device 3 receives the output information during running from the exercise analysis device 2, compares the values of various exercise indexes included in the output information during running with respective previously set target values, and reports goodness or badness of the exercise indexes to the user through sound or vibration. Thus, the user can run while recognizing the goodness or badness of each exercise index.

Further, when the exercise analysis device 2 receives the measurement end command, the exercise analysis device 2 ends the measurement of the inertial measurement unit (IMU) 10, generates user running result information (running result information: running distance and running speed), and transmits the user running result information to the reporting device 3. The reporting device 3 receives the running result information from the exercise analysis device 2, and notifies the user the running result information as a text or an image. Accordingly, the user can recognize the running result information immediately after the running end. Alternatively, the reporting device 3 may generate the running result information based on the output information during running, may notify running of the usering result information as a text or an image.

Also, data communication between the exercise analysis device 2 and the reporting device 3 may be wireless communication or may be wired communication.

Further, in the present embodiment, the exercise analysis system 1 includes a server 5 connected to a network, such as the Internet or local area network (LAN), as illustrated in FIG. 1. The information analysis device 4 is, for example, an reporting device such as a personal computer or a smart phone, and can perform data communication with the server 5 over the network. The information analysis device 4 acquires the exercise analysis information in past running of the user from the exercise analysis device 2, and transmits the exercise analysis information to the server 5 over the network. However, a device different from the information analysis device 4 may acquire the exercise analysis information from the exercise analysis device 2 and transmit the exercise analysis information to the server 5 or the exercise analysis device 2 may directly transmit the exercise analysis information to the server 5. The server 5 receives this exercise analysis information and stores the exercise analysis information in a database built in a storage unit (not illustrated). In the present embodiment, a plurality of users wear the same or different exercise analysis devices 2 and perform running, and the exercise analysis information of each user is stored in the database of the server 5.

The information analysis device 4 acquires the exercise analysis information of a plurality of users from the database of the server 5 via the network, generates analysis information from which running capabilities of the plurality of users are comparable, and displays the analysis information on a display unit (not illustrated in FIG. 1). From the analysis information displayed on the display unit of the information analysis device 4, it is possible to relatively evaluate running capability of a specific user by comparing the running capability of the specific user with running capabilities with other users, or appropriately set the target value of each exercise index. When the user sets the target value of each exercise index, the information analysis device 4 transmits the setup information of the target value of each exercise index to the reporting device 3. The reporting device 3 receives the setup information of the target values of each exercise index from the information analysis device 4, and updates each target value used for comparison with the value of each exercise index described above.

In the exercise analysis system 1, the exercise analysis device 2, the reporting device 3, and the information analysis device 4 may be separately provided, the exercise analysis device 2 and the reporting device 3 may be integrally provided and the information analysis device 4 may be separately provided, the reporting device 3 and the information analysis device 4 may be integrally provided and the exercise analysis device 2 may be separately provided, the exercise analysis device 2 and the information analysis device 4 may be integrally provided and the reporting device 3 may be separately provided, or the exercise analysis device 2, the reporting device 3, and the information analysis device 4 may be integrally provided. The exercise analysis device 2, the reporting device 3, and the information analysis device 4 may be any combination.

### 1-2. Coordinate System

Coordinate systems required in the following description are defined.

Earth centered earth fixed frame (e frame): A right-handed, three-dimensional orthogonal coordinate system in which a center of the Earth is an origin, and a z axis is parallel to a rotation axis

Navigation frame (n frame): A three-dimensional orthogonal coordinate system in which a mobile body (user) is an origin, an x axis is north, a y axis is east, and a z axis is a gravity direction

Body frame (b frame): A three-dimensional orthogonal coordinate system in which a sensor (inertial measurement unit (IMU) 10) is a reference.

Moving Frame (m frame): A right-handed, three-dimensional orthogonal coordinate system in which a mobile body (user) is an origin, and a running direction of the mobile body (user) is an x axis.

### 1-3. Motion Analysis Device

### 1-3-1. Configuration of the Exercise Analysis Device

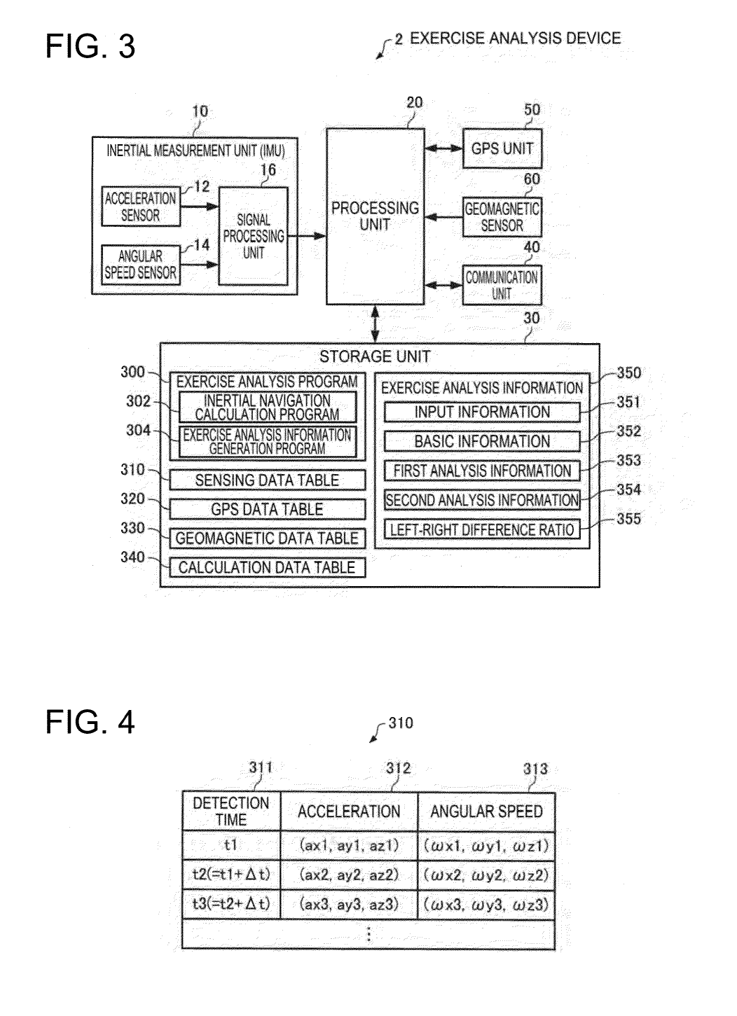

FIG. 3 is a functional block diagram illustrating an example of a configuration of an exercise analysis device 2 in the first embodiment. As illustrated in FIG. 3, the exercise analysis device 2 includes an inertial measurement unit (IMU) 10, a processing unit 20, a storage unit 30, a communication unit 40, a global positioning system (GPS) unit 50, and a geomagnetic sensor 60. However, in the exercise analysis device 2 of the present embodiment, some of these components may be removed or changed, or other components may be added.

The inertial measurement unit 10 (an example of the inertial sensor) includes an acceleration sensor 12, an angular speed sensor 14, and a signal processing unit 16.

The acceleration sensor 12 detects respective accelerations in 3-axis directions crossing one another (ideally, orthogonal to one another), and outputs a digital signal (acceleration data) according to magnitudes and directions of the detected 3-axis accelerations.

The angular speed sensor 14 detects respective angular speeds in 3-axis directions crossing one another (ideally, orthogonal to one another), and outputs a digital signal (angular speed data) according to magnitudes and directions of the detected 3-axis angular speed.

The signal processing unit 16 receives the acceleration data and the angular speed data from the acceleration sensor 12 and the angular speed sensor 14, attaches time information to the acceleration data and the angular speed data, stores the acceleration data and the angular speed data in a storage unit (not illustrated), generates sensing data obtained by causing the stored acceleration data, angular speed data, and time information to conform to a predetermined format, and outputs the sensing data to the processing unit 20.

The acceleration sensor 12 and the angular speed sensor 14 are ideally attached so that the three axes match three axes of a sensor coordinate system (b frame) relative to the inertial measurement unit 10, but an error of an attachment angle is actually generated. Therefore, the signal processing unit 16 performs a process of converting the acceleration data and the angular speed data into data of the sensor coordinate system (b-frame) using a correction parameter calculated according to the attachment angle error in advance. Also, the processing unit 20 to be described below may perform the conversion process in place of the signal processing unit 16.

Further, the signal processing unit 16 may perform a temperature correction process for the acceleration sensor 12 and the angular speed sensor 14. Also, the processing unit 20 to be described below may perform the temperature correction process in place of the signal processing unit 16, or a temperature correction function is incorporated into the acceleration sensor 12 and the angular speed sensor 14.

The acceleration sensor 12 and the angular speed sensor 14 may output analog signals. In this case, the signal processing unit 16 may perform A/D conversion on the output signal of the acceleration sensor 12 and the output signal of the angular speed sensor 14 to generate the sensing data.

The GPS unit 50 receives a GPS satellite signal transmitted from a GPS satellite which is a type of a position measurement satellite, performs position measurement calculation using the GPS satellite signal to calculate a position and a speed (a vector including magnitude and direction) of the user in the n frame, and outputs GPS data in which time information or position measurement accuracy information is attached to the position and the speed, to the processing unit 20. Also, since a method of generating the position and the speed using the GPS or a method of generating the time information is well known, a detailed description thereof will be omitted.

The geomagnetic sensor 60 detects respective geomagnetism in 3-axis directions crossing one another (ideally, orthogonal to one another), and outputs a digital signal (geomagnetic data) according to magnitudes and directions of the detected 3-axis geomagnetism. However, the geomagnetic sensor 60 may output an analog signal. In this case, the processing unit 20 may perform A/D conversion on the output signal of the geomagnetic sensor 60 to generate the geomagnetic data.

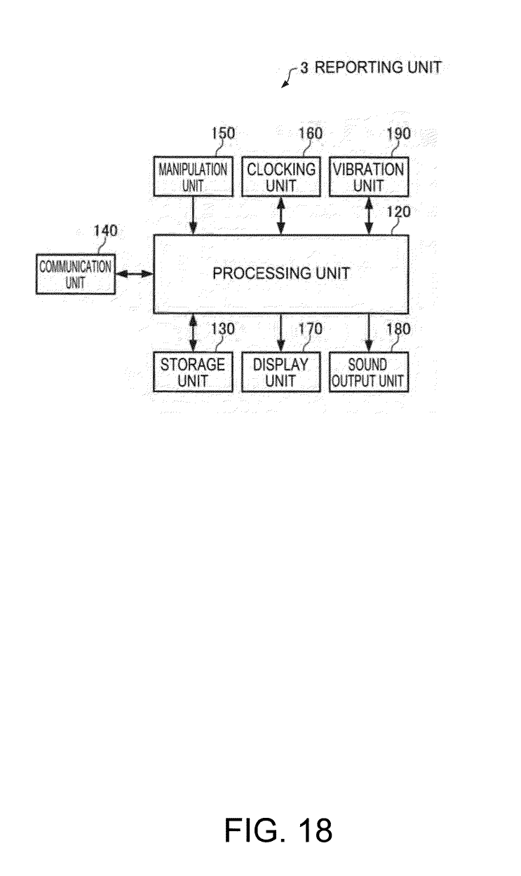

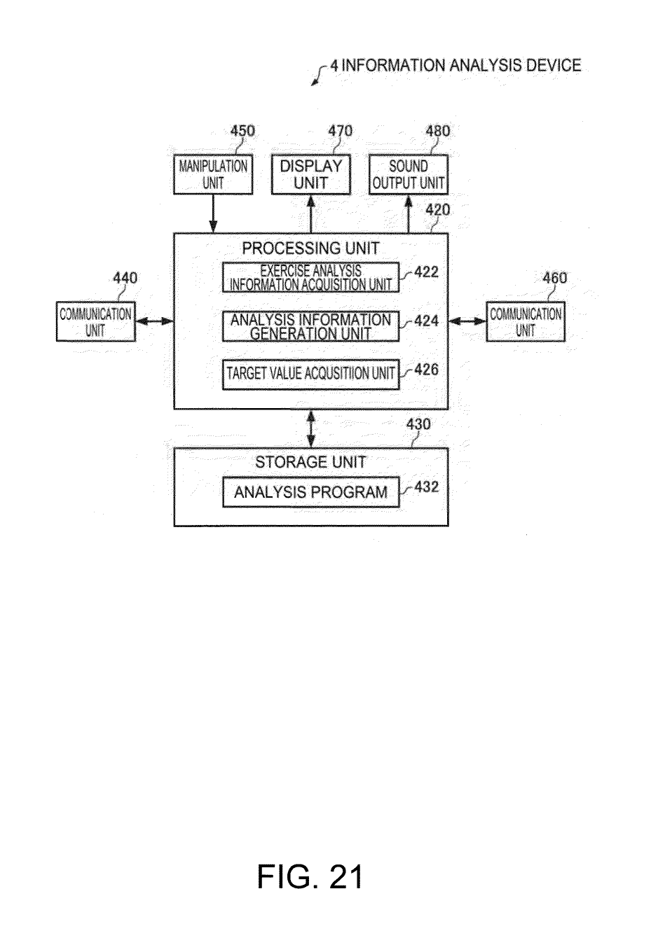

The communication unit 40 is a communication unit that performs data communication with the communication unit 140 of the reporting device 3 (see FIG. 18) or the communication unit 440 of the information analysis device 4 (see FIG. 21), and performs, for example, a process of receiving a command (for example, measurement start/measurement end command) transmitted from the communication unit 140 of the reporting device 3 and sending the command to the processing unit 20, a process of receiving the output information during running or the running result information generated by the processing unit 20 and transmitting the information to the communication unit 140 of the reporting device 3, or a process of receiving a transmission request command for exercise analysis information from the communication unit 440 of the information analysis device 4, sending the transmission request command to the processing unit 20, receiving the exercise analysis information from the processing unit 20, and transmitting the exercise analysis information to the communication unit 440 of the information analysis device 4.

The processing unit 20 includes, for example, a central processing unit (CPU), a digital signal processor (DSP), or an application specific integrated circuit (ASIC), and performs various operation processes or control processes according to various programs stored in the storage unit 30 (storage medium). In particular, when the processing unit 20 receives a measurement start command from the reporting device 3 via the communication unit 40, the processing unit 20 receives the sensing data, the GPS data, and the geomagnetic data from the inertial measurement unit 10, the GPS unit 50, and the geomagnetic sensor 60 and calculates, for example, the speed or the position of the user, and the posture angle of the torso using these data until receiving a measurement end command. Further, the processing unit 20 performs various operation processes using the calculated information, analyzes the exercise of the user to generate a variety of exercise analysis information to be described below, and stores the information in the storage unit 30. Further, the processing unit 20 performs a process of generating the output information during running or the running result information using the generated exercise analysis information, and sending the information to the communication unit 40.

Further, when the processing unit 20 receives the transmission request command for the exercise analysis information from the information analysis device 4 via the communication unit 40, the processing unit 20 performs a process of reading the exercise analysis information designated by the transmission request command from the storage unit 30, and sending the exercise analysis information to the communication unit 440 of the information analysis device 4 via the communication unit 40.

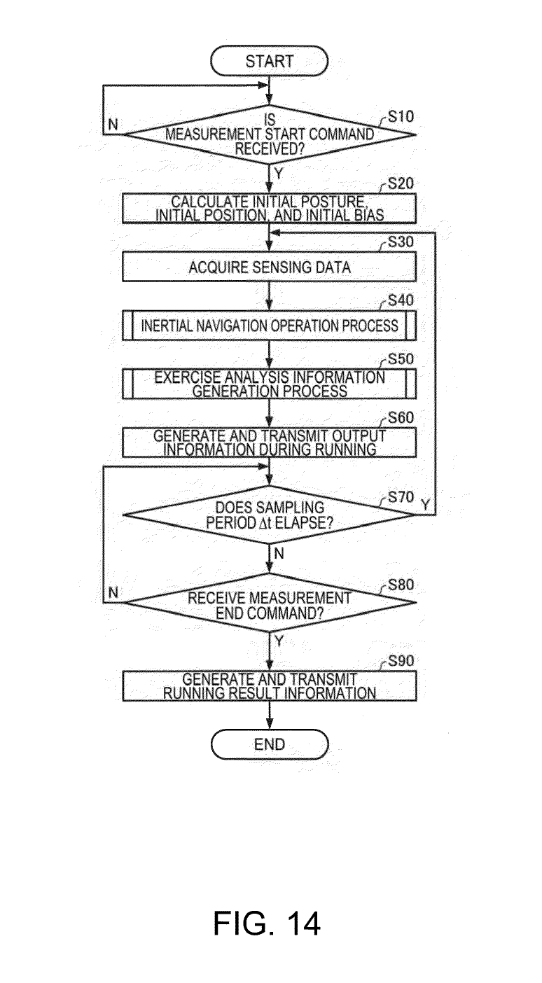

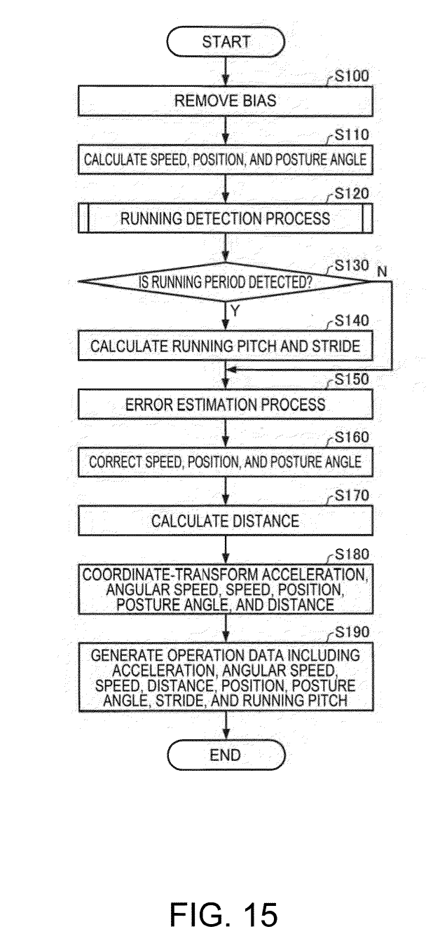

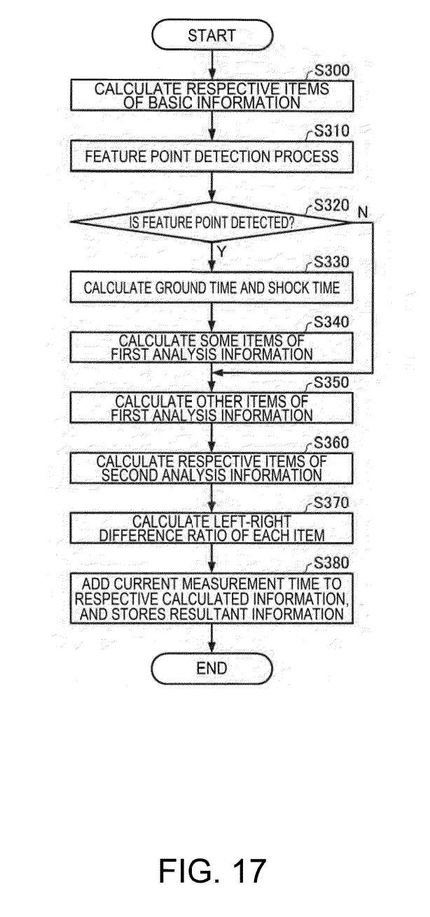

The storage unit 30 includes, for example, a recording medium that stores a program or data, such as a read only memory (ROM), a flash ROM, a hard disk, or a memory card, or a random access memory (RAM) that is a work area of the processing unit 20. A exercise analysis program 300 read by the processing unit 20, for executing the exercise analysis process (see FIG. 14) is stored in the storage unit 30 (one of the recording media). The exercise analysis program 300 includes an inertial navigation operation program 302 for executing an inertial navigation operation process (see FIG. 15), and an exercise analysis information generation program 304 for executing the exercise analysis information generation process (see FIG. 17) as subroutines.

Further, for example, a sensing data table 310, a GPS data table 320, a geomagnetic data table 330, an operation data table 340, and exercise analysis information 350 are stored in the storage unit 30.

The sensing data table 310 is a data table that stores, in time series, sensing data (detection result of the inertial measurement unit 10) that the processing unit 20 receives from the inertial measurement unit 10. FIG. 4 is a diagram illustrating an example of a configuration of the sensing data table 310. As illustrated in FIG. 4, in the sensing data table 310, the sensing data in which detection time 311 of the inertial measurement unit 10, acceleration 312 detected by the acceleration sensor 12, and angular speed 313 detected by the angular speed sensor 14 are associated with one another are arranged in time series. When the measurement starts, the processing unit 20 adds new sensing data to the sensing data table 310 each time a sampling period Δt (for example, 20 ms or 10 ms) elapses. Further, the processing unit 20 corrects the acceleration and the angular speed using an acceleration bias and an angular speed bias estimated through error estimation (which will be described below) using an extended Kalman filter, and overwrites the acceleration and the angular speed after the correction to update the sensing data table 310.

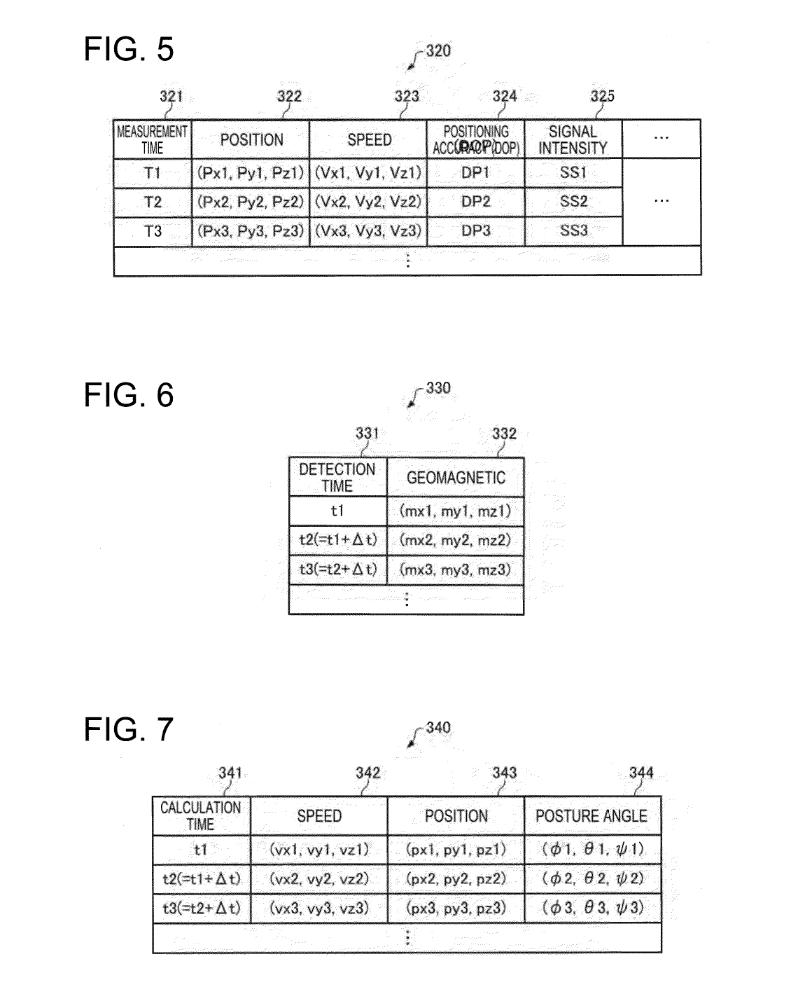

The GPS data table 320 is a data table that stores, in time series, GPS data (detection result of the GPS unit (GPS sensor) 50) that the processing unit 20 receive from the GPS unit 50. FIG. 5 is a diagram illustrating an example of a configuration of the GPS data table 320. As illustrated in FIG. 5, in the GPS data table 320, GPS data 325 in which a time 321 at which the GPS unit 50 has performed the position measurement calculation, a position 322 calculated through the position measurement calculation, speed 323 calculated through the position measurement calculation, dilution of precision; DOP) 324, and signal intensity of a received GPS satellite signal are associated is arranged in time series. When measurement starts, the processing unit 20 adds new GPS data to update the GPS data table 320 each time the processing unit 20 acquires the GPS data (for example, every second or asynchronously to an acquisition timing for the sensing data).

The geomagnetic data table 330 is a data table that stores, in time series, geomagnetic data (detection result of the geomagnetic sensor) that the processing unit 20 receives from the geomagnetic sensor 60. FIG. 6 is a diagram illustrating an example of a configuration of the geomagnetic data table 330. As illustrated in FIG. 6, in the geomagnetic data table 330, geomagnetic data in which detection time 331 of the geomagnetic sensor 60 and geomagnetic 332 detected by the geomagnetic sensor 60 are associated is arranged in time series. When the measurement starts, the processing unit 20 adds new geomagnetic data to the geomagnetic data table 330 each time the sampling period Δt (for example, 10 ms) elapses.

The operation data table 340 is a data table that stores, in time series, speed, a position, and a posture angle calculated using the sensing data by the processing unit 20. FIG. 7 is a diagram illustrating an example of a configuration of the operation data table 340. As illustrated in FIG. 7, in the data table 340, calculation data in which time 341 at which the processing unit 20 performs calculation, speed 342, position 343, and posture angle 344 are associated is arranged in time series. When the measurement starts, the processing unit 20 calculates the speed, the position, and the posture angle each time the processing unit 20 acquires new sensing data, that is, each time the sampling period Δt elapses, and adds new calculation data in the operation data table 340. Further, the processing unit 20 corrects the speed, position, and the posture angle using a speed error, a position error, and a posture angle error estimated through the error estimation using the extended Kalman filter, and overwrites the speed, the position, and the posture angle after the correction to update the operation data table 340.

The exercise analysis information 350 is a variety of information on the exercise of the user, and includes, for example, each item of input information 351, each item of basic information 352, each item of first analysis information 353, each item of second analysis information 354, and each item of a left-right difference ratio 355 generated by the processing unit 20. Details of the information on the variety of information will be described below.

### 1-3-2. Functional Configuration of the Processing Unit

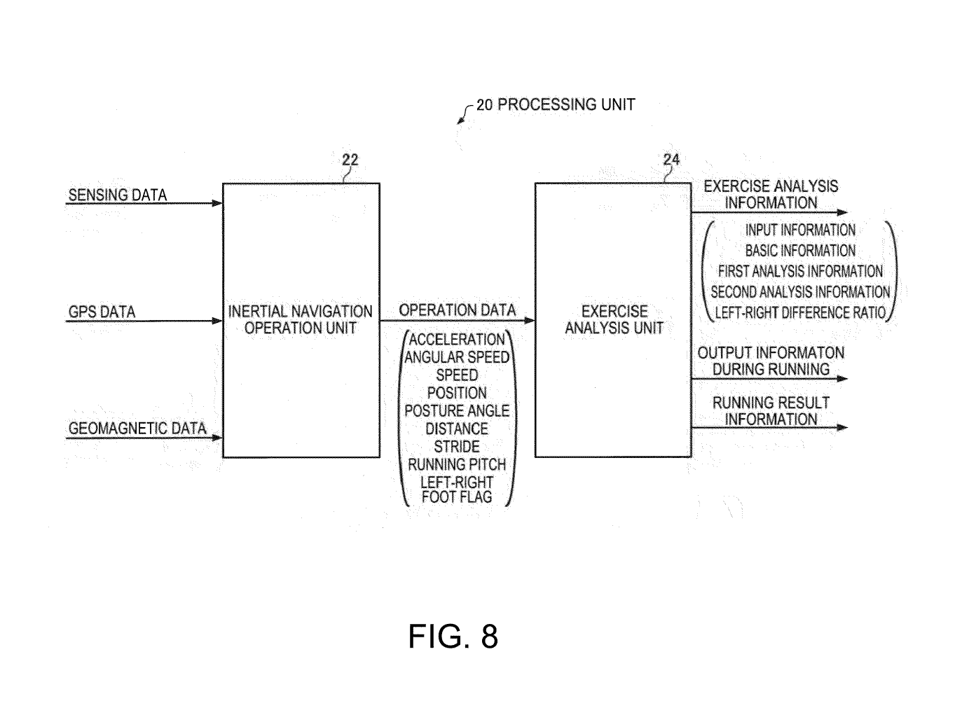

FIG. 8 is a functional block diagram illustrating an example of a configuration of the processing unit 20 of the exercise analysis device 2 in the first embodiment. In the present embodiment, the processing unit 20 executes the exercise analysis program 300 stored in the storage unit 30 to function as an inertial navigation operation unit 22 and an exercise analysis unit 24. However, the processing unit 20 may receive the exercise analysis program 300 stored in an arbitrary storage device (recording medium) via a network or the like and execute the exercise analysis program 300.

The inertial navigation operation unit 22 performs inertial navigation calculation using the sensing data (detection result of the inertial measurement unit 10), the GPS data (detection result of the GPS unit 50), and geomagnetic data (detection result of the geomagnetic sensor 60) to calculate the acceleration, the angular speed, the speed, the position, the posture angle, the distance, the stride, and the running pitch, and outputs operation data including these calculation results. The operation data output by the inertial navigation operation unit 22 is stored in a chronological order in the storage unit 30. Details of the inertial navigation operation unit 22 will be described below.

The exercise analysis unit 24 analyzes the exercise during running of the user using the operation data (operation data stored in the storage unit 30) output by the inertial navigation operation unit 22, and generates exercise analysis information (for example, input information, basic information, first analysis information, second analysis information, and a left-right difference ratio to be described below) that is information on an analysis result. The exercise analysis information generated by the exercise analysis unit 24 is stored in a chronological order in the storage unit 30 during running of the user.

Further, the exercise analysis unit 24 generates output information during running that is information output during running of the user (specifically, between start and end of measurement in the inertial measurement unit 10) using the generated exercise analysis information. The output information during running generated by the exercise analysis unit 24 is transmitted to the reporting device 3 via the communication unit 40.

Further, the exercise analysis unit 24 generates the running result information that is information on the running result at the time of running end of the user (specifically, at the time of measurement end of the inertial measurement unit 10) using the exercise analysis information generated during running. The running result information generated by the exercise analysis unit 24 is transmitted to the reporting device 3 via the communication unit 40.

### 1-3-3. Functional Configuration of the Inertial Navigation Operation Unit

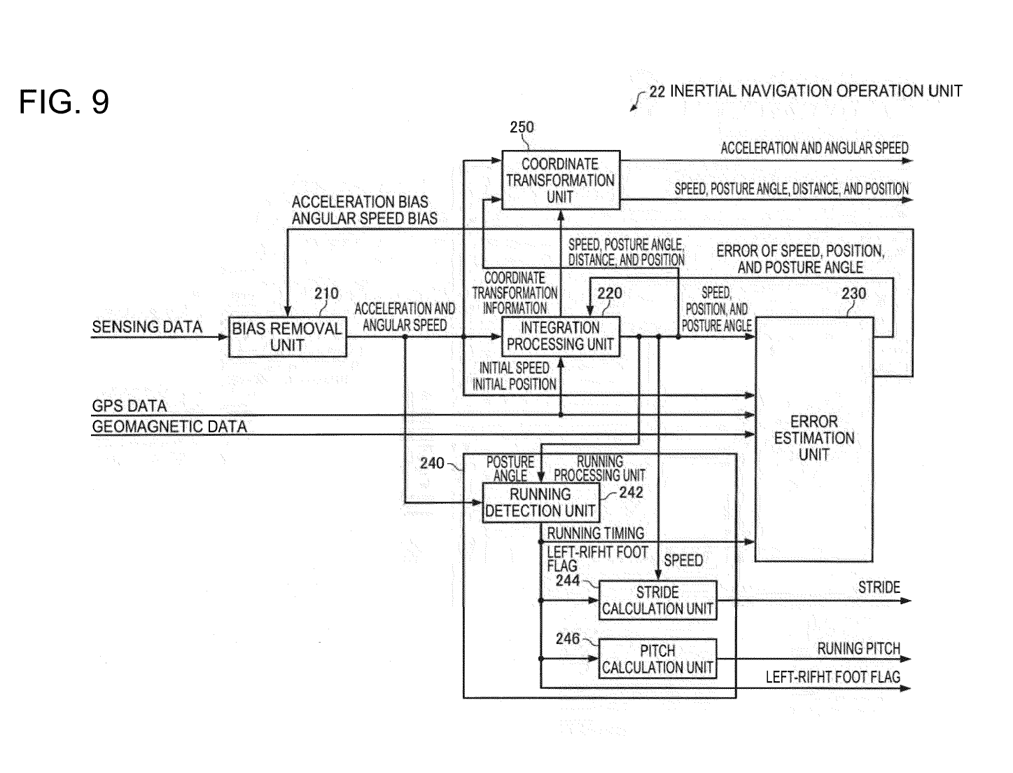

FIG. 9 is a functional block diagram illustrating an example of a configuration of the inertial navigation operation unit 22. In the present embodiment, the inertial navigation operation unit 22 includes a bias removal unit 210, an integration processing unit 220, an error estimation unit 230, a running processing unit 240, and a coordinate transformation unit 250. However, in the inertial navigation operation unit 22 of the present embodiment, some of these components may be removed or changed, or other components may be added.

The bias removal unit 210 performs a process of subtracting the acceleration bias bₐ and the angular speed bias b_{ω} estimated through the error estimation unit 230 from the 3-axis acceleration and 3-axis angular speed included in the newly acquired sensing data to correct the 3-axis acceleration and the 3-axis angular speed. Also, since there are no estimation values of the acceleration bias bₐ and the angular speed bias b_{ω} in the initial state immediately after the start of measurement, the bias removal unit 210 assumes that the initial state of the user is a resting state, and calculates the initial bias using the sensing data from the inertial measurement unit.

The integration processing unit 220 performs a process of calculating speed v^{e}, position p^{e}, and a posture angle (roll angle φ_{be}, pitch angle θ_{be}, and yaw angle ψ_{be}) of an e frame from the acceleration and the angular speed corrected by the bias removal unit 210. Specifically, the integration processing unit 220 first assumes that an initial state of the user is a resting state, sets initial speed to zero, calculates the initial speed from the speed included in the GPS data, and calculates an initial position from the position included in the GPS data. Further, the integration processing unit 220 specifies a direction of the gravitational acceleration from the 3-axis acceleration of the b frame corrected by the bias removal unit 210, calculates initial values of the roll angle φ_{be} and the pitch angle θ_{be}, calculates the initial value of the yaw angle ψ_{be} from the speed included in the GPS data, and sets the initial values as an initial posture angle of the e frame. When the GPS data cannot be obtained, the initial value of the yaw angle ψ_{be} is set to, for example, zero. Also, the integration processing unit 220 calculates an initial value of a coordinate transformation matrix (rotation matrix) C_{b}^{e} from the b frame to the e frame, which is expressed as Equation (1), from the calculated initial posture angle.

$${C}_{b}^{c} = [ \begin{matrix}cos {θ}_{be} \cdot cos {ϕ}_{be} & cos {θ}_{be} \cdot sin {ϕ}_{be} & - sin {θ}_{be} \\ sin {φ}_{be} \cdot sin {θ}_{be} \cdot cos {ϕ}_{be} - cos {φ}_{be} \cdot sin {ϕ}_{be} & sin {φ}_{be} \cdot sin {θ}_{be} \cdot sin {ϕ}_{be} + cos {φ}_{be} \cdot cos {ϕ}_{be} & sin {φ}_{be} \cdot cos {θ}_{be} \\ cos {φ}_{be} \cdot sin {θ}_{be} \cdot cos {ϕ}_{be} + sin {φ}_{be} \cdot sin {ϕ}_{be} & cos {φ}_{be} \cdot sin {θ}_{be} \cdot sin {ϕ}_{be} - sin {φ}_{be} \cdot cos {ϕ}_{be} & cos {φ}_{be} \cdot cos {θ}_{be}\end{matrix} ] \qquad ( 1 )$$

Then, the integration processing unit 220 integrates the 3-axis angular speed corrected by the bias removal unit 210 (rotation operation) to calculate a coordinate transformation matrix C_{b}^{e}, and calculates the posture angle using Equation (2).

$$[ \begin{matrix}{φ}_{be} \\ {θ}_{be} \\ {ϕ}_{be}\end{matrix} ] = [ \begin{matrix}arctan 2 ( {C}_{b}^{e} ( 2 , 3 ) , {C}_{b}^{e} ( 3 , 3 ) ) \\ - arcsin {C}_{b}^{e} ( 1 , 3 ) \\ arctan 2 ( {C}_{b}^{e} ( 1 , 2 ) , {C}_{b}^{e} ( 1 , 1 ) )\end{matrix} ] \qquad ( 2 )$$

Further, the integration processing unit 220 converts the 3-axis acceleration of the b frame corrected by the bias removal unit 210 into the 3-axis acceleration of the e frame using the coordinate transformation matrix C_{b}^{e}, and removes and integrates a gravitational acceleration component to calculate the speed v^{e} of the e frame. Further, the integration processing unit 220 integrates the speed vₑ of the e-frame to calculate the position p^{e} of the e frame.

Further, the integration processing unit 220 performs a process of correcting the speed v^{e}, the position p^{e}, and the posture angle using the speed error δv^{e}, the position error δp^{e}, and the posture angle error ε^{e} estimated by the error estimation unit 230, and a process of integrating the corrected speed v^{e} to calculate a distance.

Further, the integration processing unit 220 also calculates a coordinate transformation matrix C_{b}^{m} from the b frame to the m frame, a coordinate transformation matrix Cₑ^{m} from the e frame to the m frame, and a coordinate transformation matrix Cₑⁿ from the e frame to the n frame. These coordinate transformation matrixes are used as coordinate transformation information for a coordinate transformation process of the coordinate transformation unit 250 to be described below.

The error estimation unit 230 calculates an error of the index indicating the state of the user using, for example, the speed, the position, and the posture angle calculated by the integration processing unit 220, the acceleration or the angular speed corrected by the bias removal unit 210, GPS data, and the geomagnetic data. In the present embodiment, the error estimation unit 230 estimates the errors of the speed, the posture angle, the acceleration, the angular speed, and the position using the extended Kalman filter. That is, the error estimation unit 230 defines the state vector X as in Equation (3) by setting the error of the speed v^{e} (speed error) δv^{e} calculated by the integration processing unit 220, the error of the posture angle (posture angle error) εₑ calculated by the integration processing unit 220, the acceleration bias bₐ, the angular bias b_{ω}, and the error of the location pₑ (position error) δp^{e} calculated by the integration processing unit 220, as state variables of the extended Kalman filter.

$$X = [ \begin{matrix}δ {v}^{e} \\ {ɛ}^{e} \\ {b}_{a} \\ {b}_{ω} \\ δ {p}^{e}\end{matrix} ] \qquad ( 3 )$$

The error estimation unit 230 predicts a state variable included in the state vector X using a prediction equation of the extended Kalman filter. The prediction equation of the extended Kalman filter is expressed by Equation (4). In Equation (4), a matrix Φ is a matrix that associates a previous state vector X with a current state vector X, and some of elements of the matrix are designed to change every moment while reflecting, for example, the posture angle or the position. Further, Q is a matrix representing process noise, and each element of Q is set to an appropriate value in advance. Further, P is an error covariance matrix of the state variable.

X=ΦX

P=ΦPΦ^{T}+Q (4)

Further, the error estimation unit 230 updates (corrects) the predicted state variable using the updating equation of the extended Kalman filter. The updating equation of the extended Kalman filter is expressed as Equation (5). Z and H are an observation vector and an observation matrix, respectively. The updating equation (5) shows that the state vector X is corrected using a difference between an actual observation vector Z and a vector HX predicted from the state vector X. R is a covariance matrix of the observation error, and may be a predetermined constant value or may be dynamically changed. K indicates a Kalman gain, and K increases as R decreases. From Equation (5), as K increases (R decreases), an amount of correction of the state vector X increases and P correspondingly decreases.

K=PH^{T}(HPH^{T}+R)⁻¹

X=X+K(Z−HX)

P=(I−KH)P (5)

Examples of an error estimation method (method of estimating the state vector X) include the following methods.

### Error Estimation Method Using Correction Based on the Posture Angle Error:

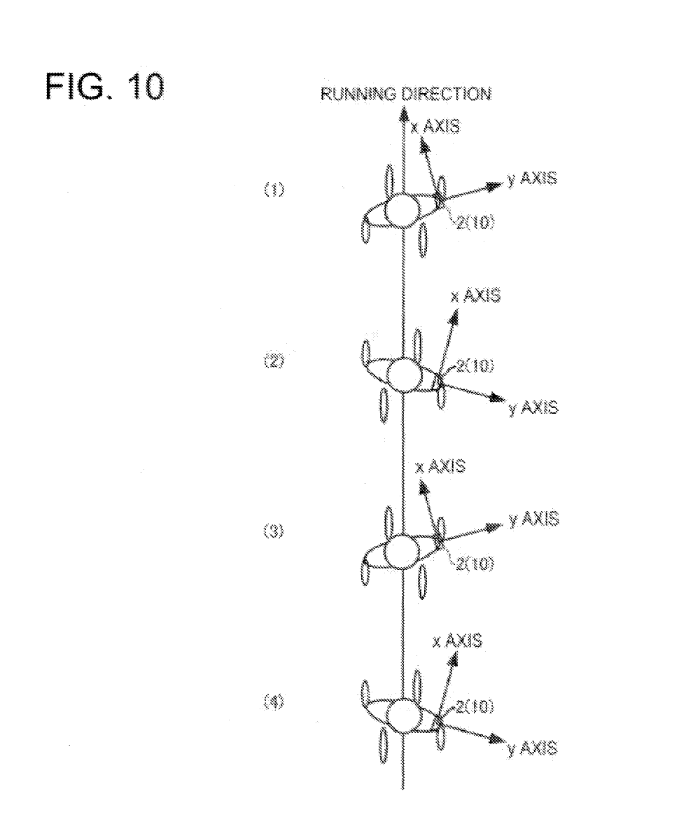

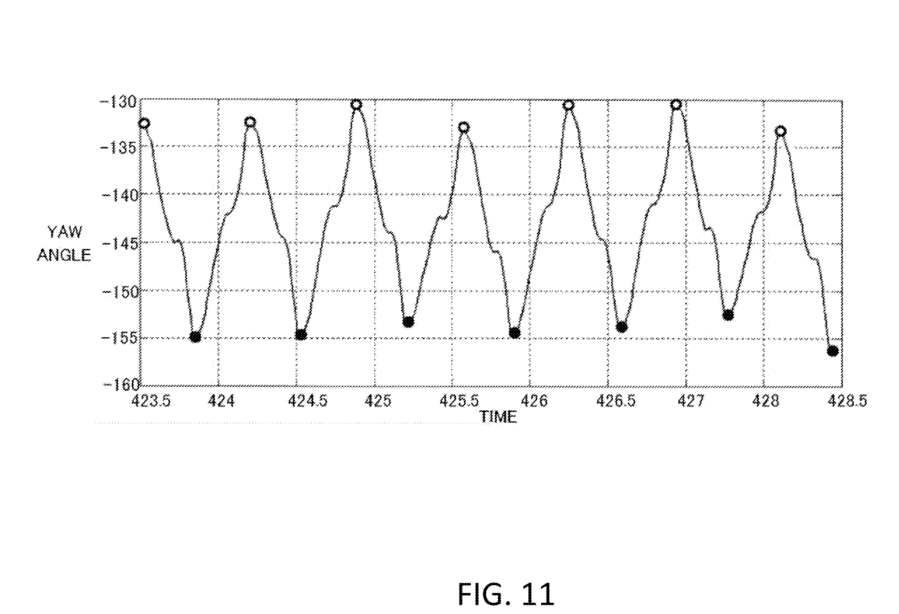

FIG. 10 is a diagram illustrating an overhead view of the movement of the user when a user wearing the exercise analysis device 2 on the right waist performs a running operation (straight running) Further, FIG. 11 is a diagram illustrating an example of a yaw angle (azimuth angle) calculated from the detection result of the inertial measurement unit 10 when the user performs a running operation (straight running) A horizontal axis indicates time and a vertical axis indicates a yaw angle (azimuth angle).

With the running operation of the user, the posture of the inertial measurement unit 10 with respect to the user changes at any time. In a state in which the user steps forward with a left foot, the inertial measurement unit 10 has a posture inclined to the left with respect to the running direction (x axis of the m frame), as illustrated in (1) or (3) in FIG. 10. On the other hand, in a state in which the user steps forward with a right leg, the inertial measurement unit 10 has a posture inclined to the right side with respect to the running direction (x axis of the m frame) as illustrated in (2) or (4) in FIG. 10. That is, the posture of the inertial measurement unit 10 periodically changes in every two steps of left and right steps with the running operation of the user. In FIG. 11, for example, the yaw angle is maximized in a state in which the user steps forward with the right leg (∘ in FIG. 11), and the yaw angle is minimized in a state in which the user steps forward with the left leg ( in FIG. 11). Therefore, the error can be estimated on the assumption that a previous (before two steps) posture angle and a current posture angle are equal, and the previous posture angle is a true posture. In this method, the observation vector Z in Equation (5) is a difference between the previous posture angle and the current posture angle calculated by the integration processing unit 220, and the state vector X is corrected based on a difference the posture angle error εₑ and the observation value using the updating equation (5), and the error is estimated.

### Error Estimation Method Using Correction Based on the Angular Speed Bias:

This is a method of estimating the error on the assumption that a previous (before two steps) posture angle is equal to the current posture angle, but it is not necessary for the previous posture angle to be a true posture. In this method, the observation vector Z in Equation (5) is an angular speed bias calculated from the previous posture angle and the current posture angle calculated by the integration processing unit 220. Using the updating equation (5), the state vector X is corrected based on a difference between the angular speed bias be) and the observation value, and the error is estimated.

### Error Estimation Method Using Correction Based on an Azimuth Angle Error:

This is a method of estimating the error on the assumption that a previous (before two steps) yaw angle (azimuth angle) is equal to a current yaw angle (azimuth angle), and the previous yaw angle (azimuth angle) is a true yaw angle (azimuth angle). In this method, the observation vector Z is a difference between the previous yaw angle and the current yaw angle calculated by the integration processing unit 220. Using the updating equation (5), the state vector X is corrected based on a difference between the azimuth angle error ε_{z}^{e} and the observation value, and the error is estimated.

### Error Estimation Method Using Correction Based on Stop:

This is a method of estimating the error on the assumption that the speed is zero at the time of stop. In this method, the observation vector Z is a difference between the speed v^{e} calculated by the integration processing unit 220 and zero. Using the updating equation (5), the state vector X is corrected based on the speed error δv^{e}, and the error is estimated.

### Error Estimation Method Using Correction Based on Rest:

This is a method of estimating the error on the assumption that the speed is zero at rest, and a posture change is zero. In this method, the observation vector Z is an error of the speed v^{e} calculated by the integration processing unit 220, and a difference between the previous posture angle and the current posture angle calculated by the integration processing unit 220. Using the updating equation (5), the state vector X is corrected based on the speed error δv^{e} and the posture angle error ε^{e}, and the error is estimated.

### Error Estimation Method Using Correction Based on the GPS Observations Value:

This is a method of estimating the error on the assumption that the speed v^{e}, the position p^{e}, or the yaw angle ψ_{be} calculated by the integration processing unit 220 is equal to the speed, position, or azimuth angle (the speed, position, or azimuth angle after conversion into the e frame) calculated from the GPS data. In this method, the observation vector Z is a difference between the speed, position, or yaw angle calculated by the integration processing unit 220 and the speed, position, or azimuth angle calculated from the GPS data. Using the updating equation (5), the state vector X is corrected based on a difference between the speed error δv^{e}, the position error δp^{e}, or the azimuth angle error ε_{z}^{e} and the observation value, and the error is estimated.

### Error Estimation Method Using Correction Based on the Observation Value of the Geomagnetic Sensor:

This is a method of estimating the error on the assumption that the yaw angle ψ_{be} calculated by the integration processing unit 220 is equal to the azimuth angle (azimuth angle after conversion into the e-frame) calculated from the geomagnetic sensor. In this method, the observation vector Z is a difference between the yaw angle calculated by the integration processing unit 220 and the azimuth angle calculated from the geomagnetic data. Using the updating equation (5), the state vector X is corrected based on the difference between the azimuth angle error ε_{z}^{e} and the observation value, and the error is the estimated.

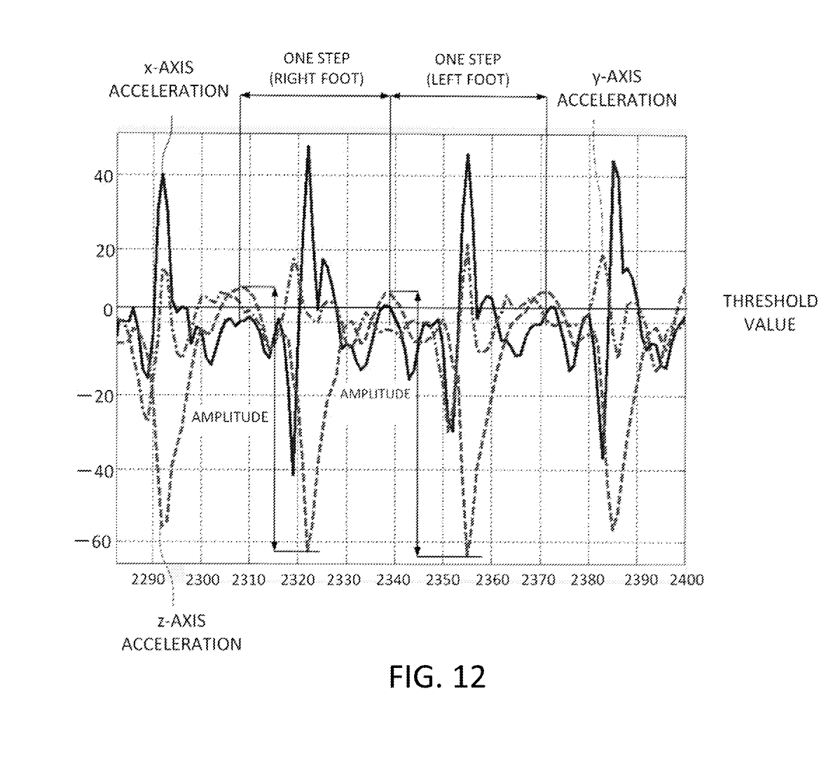

Referring back to FIG. 9, the running processing unit 240 includes a running detection unit 242, a stride calculation unit 244, and a pitch calculation unit 246. The running detection unit 242 performs a process of detecting the running period (running timing) of the user using a detection result of the inertial measurement unit 10 (specifically, the sensing data corrected by the bias removal unit 210). As described in FIGS. 10 and 11, since the posture of the user changes periodically (every two steps (one left step and one right step)) when the user runs, the acceleration detected by the inertial measurement unit 10 also changes periodically. FIG. 12 is a diagram illustrating an example of a 3-axis acceleration detected by the inertial measurement unit 10 when the user runs. In FIG. 12, a horizontal axis indicates time and a vertical axis indicates an acceleration value. As illustrated in FIG. 12, the 3-axis acceleration periodically changes, and in particular, the z-axis (axis in a direction of gravity) acceleration, can be seen as regularly changing with a periodicity. This z-axis acceleration reflects the acceleration of a vertical movement of the user, and a period from a time at which the z-axis acceleration becomes a maximum value equal to or greater than a predetermined threshold value to a time at which the z-axis acceleration next becomes the maximum value equal to or greater than the threshold value corresponds to a period of one step.

Therefore, in the present embodiment, the running detection unit 242 detects the running period each time the z-axis acceleration (corresponding to the acceleration of the vertical movement of the user) detected by the inertial measurement unit 10 becomes a maximum value equal to or greater than a predetermined threshold value. That is, the running detection unit 242 outputs a timing signal indicating that the running detection unit 242 detects the running period each time the z-axis acceleration becomes the maximum value equal to or greater than the predetermined threshold value. In fact, since a high-frequency noise component is included in the 3-axis acceleration detected by the inertial measurement unit 10, the running detection unit 242 detects the running period using the z-axis acceleration passing through a low pass filter so that noise is removed.

Further, the running detection unit 242 determines whether the detected running period is a left running period or a right running period, and outputs a right and left leg flag (for example, ON for the right foot and OFF for left foot) indicating whether the detected running period is a left running period or a right running period. For example, as illustrated in FIG. 11, since the yaw angle is maximized (∘ in FIG. 11) in a state in which the right leg steps forward, and the yaw angle is minimized ( in FIG. 11) in a state in which the left leg steps forward, the running detection unit 242 can determine whether the running cycle is a left running cycle or a right running cycle using the posture angle (particularly, the yaw angle) calculated by the integration processing unit 220. Further, as illustrated in FIG. 10, when viewed from an overhead side of the user, the inertial measurement unit 10 rotates clockwise from a state in which the user steps forward with the left foot (state (1) or (3) in FIG. 10) to a state in which the user steps forward with the right foot (state (2) or (4) in FIG. 10), and reversely rotates in a counterclockwise from the state in which the user steps forward with the right foot to the state in which the user steps forward with the left foot. Thus, for example, the running detection unit 242 can also determine whether the running period is a left running period or a right running period based on a polarity of the z-axis angular speed. In this case, since a high-frequency noise component is, in fact, included in the 3-axis angular speed detected by the inertial measurement unit 10, the running detection unit 242 determines whether the running period is a left running period or a right running period using the z-axis angular speed passing through a low pass filter so that noise is removed.

The stride calculation unit 244 performs a process of calculating right and left strides using a timing signal of the running period output by the running detecting unit 242, the left and right foot flag, and the speed or the position calculated by the integration processing unit 220, and outputs the strides as right and left strides. That is, the stride calculation unit 244 integrates the speed in a period from the start of the running period to the start of the next running period, that is, a sampling period Δt (calculates a difference between a position at the time of start of the running period and a position at the time of start of the next running period) to calculate the stride, and outputs the stride as a stride.

The pitch calculation unit 246 performs a process of calculating the number of steps for 1 minute using the timing signal having a running period output by the running detection unit 242, and outputting the number of steps as a running pitch. That is, the pitch calculation unit 246, for example, takes a reciprocal of the running period to calculate the number of steps per second, and multiplies the number of steps by 60 to calculate the number of steps (running pitch) for 1 minute.

The coordinate transformation unit 250 performs a coordinate transformation process of transforming the 3-axis acceleration and the 3-axis angular speed of the b frame corrected by the bias removal unit 210 into the 3-axis acceleration and the 3-axis angular speed of the m frame using the coordinate transformation information (coordinate transformation matrix C_{b}^{m}) from the b frame to the m-frame calculated by the integration processing unit 220. Further, the coordinate transformation unit 250 performs a coordinate transformation process of transforming the speed in the 3-axis direction, the posture angle in the 3-axis direction, and the distance in the 3-axis direction of the e frame calculated by the integration processing unit 220 into the speed in the 3-axis direction, the posture angle in the 3-axis direction, and the distance in the 3-axis direction of the m frame using the coordinate transformation information (coordinate transformation matrix Cₑ^{m}) from the e frame to the m-frame calculated by the integration processing unit 220. Further, the coordinate transformation unit 250 performs a coordinate transformation process of transforming a position of the e frame calculated by the integration processing unit 220 into a position of the n frame using the coordinate transformation information (coordinate transformation matrix Cₑⁿ) from the e frame to the n frame calculated by the integration processing unit 220.

Also, the inertial navigation operation unit 22 outputs operation data including respective information of the acceleration, the angular speed, the speed, the position, the posture angle, and the distance after coordinate transformation in the coordinate transformation unit 250, and the stride, the running pitch, and left and right foot flags calculated by the running processing unit 240 (stores the information in the storage unit 30).

### 1-3-4. Functional Configuration of the Exercise Analysis Device

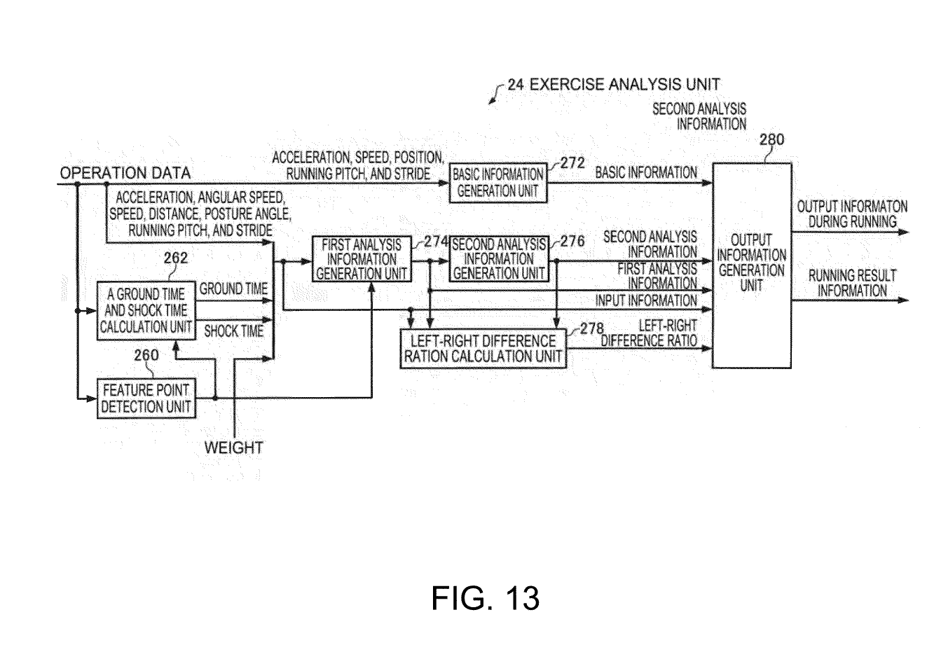

FIG. 13 is a functional block diagram illustrating an example of a configuration of the exercise analysis unit 24 in the first embodiment. In the present embodiment, the exercise analysis unit 24 includes a feature point detection unit 260, a ground time and shock time calculation unit 262, a basic information generation unit 272, a first analysis information generation unit 274, a second analysis information generation unit 276, a left-right difference ratio calculation unit 278, and an output information generation unit 280. However, in the exercise analysis unit 24 of the present embodiment, some of these components may be removed or changed, or other components may be added.

The feature point detection unit 260 performs a process of detecting a feature point in the running operation of the user using the operation data. Examples of the feature point in the running operation of the user includes landing (for example, a time when a portion of a sole of the foot arrives at the ground, a time when the entire sole of the foot arrives on the ground, any time point while a heel of the foot first arrives and then a toe thereof is separated, any time point while the toe of the foot first arrives and then the heel thereof is separated, and a time while the entire sole of the foot arrives may be appropriately set), depression (a state which most weight is applied to the foot), and separation from ground (also referred to as kicking; a time when a portion of the sole of the foot is separated from the ground, a time when the entire sole of the foot is separated from the ground, any time point while a heel of the foot first arrives and then a toe thereof is separated, and any time point while the toe of the foot first arrives and then separated is separated may be appropriately set. Specifically, the feature point detection unit 260 separately detects the feature point in the running period of the right leg and the feature point in the running period of the left foot using the right and left leg flag included in the operation data. For example, the feature point detection unit 260 can detect the landing at a timing at which the acceleration in the vertical direction (detection value of the z axis of the acceleration sensor) changes from a positive value to a negative value, detect depression at a time point at which the acceleration in a running direction becomes a peak after the acceleration in the vertical direction becomes a peak in a negative direction after landing, and detect separation from ground (kicking) at a time point at which the acceleration in the vertical direction changes from a negative value to a positive value.

The ground time and shock time calculation unit 262 performs a process of calculating respective values of the ground time and the shock time based on a timing at which the feature point detection unit 260 detects the feature point using the operation data. Specifically, the ground time and shock time calculation unit 262 determines whether current operation data is operation data of the running period of the right foot or operation data of the running period of the left foot from the left and right foot flag included in the calculation data, and calculates the respective values of the ground time and the shock time in the running period of the right foot and the running period of the left foot based on a time point at which the feature point detection unit 260 detects the feature point. Definitions and calculation methods of the ground time and the shock time will be described below in detail.

The basic information generation unit 272 performs a process of generating basic information on the exercise of the user using the information on the acceleration, speed, position, stride, and running pitch included in the operation data. Here, the basic information includes respective items of the running pitch, the stride, the running speed, altitude, running distance, and running time (lap time). Specifically, the basic information generation unit 272 outputs the running pitch and the stride included in the calculation data as the running pitch and the stride of the basic information. Further, the basic information generation unit 272 calculates, for example, current values of the running speed, the altitude, the running distance, and the running time (lap time) or average values thereof during running using some or all of the acceleration, the speed, the position, the running pitch, and the stride included in the operation data.

The first analysis information generation unit 274 analyzes user's exercise at which the feature point detection unit 260 detects the feature point using the input information, and performs a process of generating the first analysis information.

Here, the input information includes respective items of acceleration in a running direction, speed in the running direction, distance in the running direction, acceleration in the vertical direction, speed in the vertical direction, distance in the vertical direction, acceleration in a horizontal direction, horizontal direction speed, distance in the horizontal direction, posture angle (roll angle, pitch angle, and yaw angle), angular speed (roll direction, pitch direction, and yaw direction), running pitch, stride, ground time, shock time, and weight. The body weight is input by the user, ground time and shock time are calculated by the ground time and shock time calculation unit 262, and other items are included in the calculation data.

Further, the first analysis information includes respective items of amounts of brake at the time of landing (amount of brake 1 at the time of landing, and amount of brake 2 at the time of landing), directly-under landing rates (directly-under landing rate 1, directly-under landing rate 2, and directly-under landing rate 3), propulsion power (propulsion power 1, and propulsion power 2), propulsion efficiency (propulsion efficiency 1, propulsion efficiency 2, propulsion efficiency 3, and propulsion efficiency 4), an amount of energy consumption, landing shock, running capability, an anteversion angle, a degree of timing matching, and a flow of a leg. Each item of the first analysis information is an item indicating a running state (an example of an exercise state) of the user. A definition and a calculation method for each item of the first analysis information will be described below in detail.

Further, the first analysis information generation unit 274 calculates the value of each item of the first analysis information for left and right of the body of the user. Specifically, the first analysis information generation unit 274 calculates each item included in the first analysis information in the running period of the right foot and the running period of the left foot according to whether the feature point detection unit 260 detects the feature point in the running period of the right foot or the feature point in the running period of the left foot. Further, the first analysis information generation unit 274 also calculates left and right average values or a sum value for each item included in the first analysis information.

The second analysis information generation unit 276 performs a process of generating the second analysis information using the first analysis information calculated by the first analysis information generation unit 274. Here, the second analysis information includes respective items of energy loss, energy efficiency, and a load on the body. A definition and a calculation method for each item of the second analysis information will be described below in detail. The second analysis information generation unit 276 calculates values of the respective items of the second analysis information in the running period of the right foot and the running period of the left foot. Further, the second analysis information generation unit 276 also calculates the left and right average values or the sum value for each item included in the second analysis information.

The left-right difference ratio calculation unit 278 performs a process of calculating a left-right difference ratio that is an index indicating left-right balance of the body of the user using a value in the running period of the right foot and a value in the running period of the left foot for the running pitch, the stride, the ground time, and the shock time included in the input information, all items of the first analysis information, and all items of the second analysis information. A definition and a calculation method for the left-right difference ratio will be described below in detail.

The output information generation unit 280 performs a process of generating the output information during running that is information output during running of the user using, for example, the basic information, the input information, the first analysis information, the second analysis information, and the left-right difference ratio. “Running pitch”, “stride”, “ground time”, and “shock time” included in the input information, all items of the first analysis information, all items of the second analysis information, and the left-right difference ratio are exercise indexes used for evaluation of the running skill of the user, and the output information during running includes information on values of some or all of the exercise indexes. The exercise indexes included in the output information during running may be determined in advance, or may be selected by the user manipulating the reporting device 3. Further, the output information during running may include some or all of running speed, altitude, a running distance, and a running time (lap time) included in the basic information.

Further, the output information generation unit 280 generates running result information that is information on a running result of the user using, for example, the basic information, the input information, the first analysis information, and the second analysis information, and the left-right difference ratio. For example, the output information generation unit 280 may generate the running result information including, for example, information on an average value of each exercise index during running of the user (during measurement of the inertial measurement unit 10). Further, the running result information may include some or all of the running speed, the altitude, the running distance, and the running time (lap time).

The output information generation unit 280 transmits the output information during running to the reporting device 3 via the communication unit 40 during running of the user, and transmits the running result information to the reporting device 3 at the time of running end of the user.

### 1-3-5. Input Information

Hereinafter, respective items of input information will be described in detail.

### Acceleration in the Running Direction, Acceleration in Vertical Direction, and Acceleration in Horizontal Direction

A “running direction” is a running direction of the user (x-axis direction of the m frame), a “vertical direction” is a vertical direction (z-axis direction of the m frame), and a “horizontal direction” is a direction (y-axis direction of the m frame) perpendicular to the running direction and the vertical direction. The acceleration in the running direction, the acceleration in the vertical direction, and the acceleration in the horizontal direction are acceleration in the x-axis direction, acceleration in the z-axis direction, and acceleration in the y-axis direction of the m frame, respectively, and are calculated by the coordinate transformation unit 250.

### Speed in Running Direction, Speed in Vertical Direction, and Speed in Horizontal Direction

Speed in a running direction, speed in a vertical direction, and speed in a horizontal direction are speed in an x-axis direction, speed in a z-axis direction, and speed in a y-axis direction of the m frame, respectively, and are calculated by the coordinate transformation unit 250. Alternatively, acceleration in the running direction, acceleration in a vertical direction, and acceleration in a horizontal direction can be integrated to calculate the speed in the running direction, the speed in the vertical direction, and the speed in the horizontal direction, respectively.

### Angular Speed (Roll Direction, Pitch Direction, and Yaw Direction)

Angular speed in a roll direction, angular speed in a pitch direction, and angular speed in a yaw direction are angular speed in an x-axis direction, angular speed in a y-axis direction, and angular speed in a z-axis direction of the m frame, respectively, and are calculated by the coordinate transformation unit 250.

### Posture Angle (Roll Angle, Pitch Angle, and Yaw Angle)

An roll angle, a pitch angle, and a yaw angle are a posture angle in an x-axis direction, a posture angle in a y-axis direction, and a posture angle in a z-axis direction of the m frame that are output, respectively and calculated by the coordinate transformation unit 250. Alternatively, an angular speed in the roll direction, an angular speed in the pitch direction, and the angular speed in the yaw direction can be integrated (rotation operation) to calculate the roll angle, the pitch angle, and the yaw angle.

### Distance in Running Direction, Distance in the Vertical Direction, Distance in the Horizontal Direction

A distance in the running direction, a distance in the vertical direction, and a distance in the horizontal direction are a movement distance in the x-axis direction, a movement distance in the z-axis direction, and a movement distance in the y-axis direction of the m frame from a desired position (for example, a position immediately before the user starts running), respectively, and are calculated by the coordinate transformation unit 250.

### Running Pitch

A running pitch is an exercise index defined as the number of steps per minute and is calculated by the pitch calculation unit 246. Alternatively, the running pitch can be calculated by dividing the distance in the running direction for one minute by the stride.

### Stride

The stride is an exercise index defined as a stride of one step, and is calculated by the stride calculation unit 244. Alternatively, the stride can be calculated by dividing the distance in the running direction for one minute by the running pitch.

### Ground Time

A ground time is an exercise index defined as a time taken from landing to separation from ground (kicking), and is calculated by the ground time and shock time calculation unit 262. The separation from ground (kicking) is a time when the toe is separated from the ground. Also, since the ground time has high correlation with the running speed, the ground time can also be used as the running capability of the first analysis information.

### Shock Time

A shock time is an exercise index defined as a time at which shock generated due to landing is applied to the body, and is calculated by the ground time and shock time calculation unit 262. The shock time can be calculated as shock time=(time at which acceleration in a running direction in one step is minimized−time of landing).

### Weight

A weight is a weight of the user, and a numerical value of the weight is input by the user manipulating the manipulation unit 150 (see FIG. 18) before running.

### 1-3-6. First Analysis Information

Hereinafter, respective items of the first analysis information calculated by the first analysis information generation unit 274 will be described in detail.

### Amount of Brake 1 at the Time of Landing

An amount of brake 1 at the time of landing is an exercise index defined as an amount of speed decreased due to landing, and can be calculated as an amount of brake 1 at the time of landing=(speed in the running direction before landing−minimum speed in the running direction after landing). The speed in the running direction is decreased due to landing, and a lowest point of the speed in the running direction after landing in one step is the lowest speed in the running direction.

### Amount of Brake 2 at the Time of Landing

The amount of brake 2 at the time of landing is an exercise index defined as an amount of lowest acceleration in a negative running direction generated due to landing, and matches minimum acceleration in the running direction after landing in one step. The lowest point of the acceleration in the running direction after landing in one step is the lowest acceleration in the running direction.

### Directly-Under Landing Rate 1

A directly-under landing rate 1 is an exercise index indicating whether the player lands under the body. When the player can land directly under the body, the amount of brake decreases and the player can efficiently run. Since the amount of brake normally increases according to the speed, the amount of brake is an insufficient index, but since directly-under landing rate 1 is an index expressed at a rate, the same evaluation is possible according to the directly-under landing rate 1 even when the speed changes. When α=arctan (acceleration in a running direction at the time of landing/acceleration in a vertical direction at the time of landing) using the acceleration in the running direction (negative acceleration) and the acceleration in the vertical direction at the time of landing, directly-under landing rate 1 can be calculated as directly-under landing rate 1=cos α×100 (%). Alternatively, an ideal angle α′ can be calculated using data of a plurality of persons who fast run, and directly-under landing rate 1 can be calculated as directly-under landing rate 1={1−|(α′−α)/α′|}×100(%).

### Directly-Under Landing Rate 2

A directly-under landing rate 2 is an exercise index indicating whether the player lands directly under the body, using a degree of speed decrease, and is calculated as directly-under landing rate 2=(minimum speed in the running direction after landing/speed in the running direction directly before landing)×100(%).

### Directly-Under Landing Rate 3

Directly-under landing rate 3 is an exercise index indicating whether the player lands directly under the body using a distance or time from landing to the foot coming directly under the body. The directly-under landing rate 3 can be calculated as directly-under landing rate 3=(distance in the running direction when the foot comes directly under the body−distance in the running direction at the time of landing), or as directly-under landing rate 3=(time when the foot comes directly under the body−time of landing). After landing (point at which the acceleration in the vertical direction is changed from a positive value to a negative value), there is a timing at which the acceleration in the vertical direction becomes a peak in a negative direction, and this time can be determined to be a timing (time) at which the foot comes directly under the body.

Also, in addition, the directly-under landing rate 3 may be defined as directly-under landing rate 3=arctan (distance from landing to the foot coming directly under the body/height of waist). Alternatively, the directly-under landing rate 3 may be defined as directly-under landing rate 3=(1−distance from landing to the foot coming directly under the body/distance of movement from landing to kicking)×100(%) (a ratio of the distance from landing to the foot coming directly under the body to a distance of movement while the foot is grounded). Alternatively, the directly-under landing rate 3 may be defined as directly-under landing rate 3=(1−time from landing to the foot coming directly under the body/time of movement from landing to kicking)×100(%) (a ratio of the time from landing to the foot coming directly under the body to time of movement while the foot is grounded).

### Propulsion Force 1

Propulsion force 1 is an exercise index defined as amount of speed increasing in the running direction by kicking the ground, and can be calculated using propulsion power 1=(maximum speed in running direction after kicking−minimum speed in running direction before kicking.

### Propulsion Force 2

Propulsion force 2 is an exercise index defined as maximum acceleration in a positive running direction generated by kicking, and matches maximum acceleration in the running direction after kicking in one step.

### Propulsion Efficiency 1

Propulsion efficiency 1 is an exercise index indicating whether kicking force efficiently becomes propulsion power. When wasteful vertical movement and wasteful horizontal movement disappear, efficient running is possible. Typically, since the vertical movement and the horizontal movement increase according to the speed, the vertical movement and the horizontal movement are insufficient as exercise indexes, but since propulsion efficiency 1 is the exercise index expressed at a rate, the same evaluation is possible according to propulsion efficiency 1 even when the speed changes. The propulsion efficiency 1 is calculated in each of the vertical direction and the horizontal direction. When γ=arctan (acceleration in the vertical direction at the time of kicking/acceleration in a running direction at the time of kicking) using the acceleration in the vertical direction and the acceleration in a running direction at the time of kicking, propulsion efficiency 1 in the vertical direction can be calculated as the propulsion efficiency 1 in the vertical direction=cos γ×100(%). Alternatively, an ideal angle γ′ can be calculated using data of a plurality of persons who fast run, and propulsion efficiency 1 in the vertical direction can also be calculated as propulsion efficiency 1 in the vertical direction={1−|(γ′−γ)/γ′|}×100(%). Similarly, when δ=arctan (acceleration in a horizontal direction at the time of kicking/acceleration in a running direction at the time of kicking) using the acceleration in a horizontal direction and the acceleration in a running direction at the time of kicking, propulsion efficiency 1 in the horizontal direction can be calculated as the propulsion efficiency 1 in the horizontal direction=cos δ×100(%). Alternatively, an ideal angle δ′ can be calculated using data of a plurality of persons who fast run, and propulsion efficiency 1 in the horizontal direction can be calculated as propulsion efficiency 1 in a horizontal direction={1−|(δ−δ)/δ′|}×100(%).

Also, in addition, the propulsion efficiency 1 in the vertical direction can also be calculated by replacing γ with arctan (speed in the vertical direction at the time of kicking/speed in the running direction at the time of kicking) Similarly, the propulsion efficiency 1 in the horizontal direction can also be calculated by replacing δ with arctan (speed in the horizontal direction at the time of kicking/speed in the running direction at the time of kicking).

### Propulsion Efficiency 2

Propulsion efficiency 2 is an exercise index indicating whether the kicking force efficiently becomes propulsion power, using an angle of the acceleration at the time of depression. When ξ=arctan (acceleration in the vertical direction at the time of depression/acceleration in a running direction at the time of depression) using the acceleration in the vertical direction and the acceleration in a running direction at the time of depression, propulsion efficiency 2 in the vertical direction can be calculated as the propulsion efficiency 2 in the vertical direction=cos ξ×100(%). Alternatively, an ideal angle ξ′ can be calculated using data of a plurality of persons who fast run, and propulsion efficiency 2 in the vertical direction can also be calculated as propulsion efficiency 2 in the vertical direction={1−|(ξ′−ξ)/ξ′|}×100(%). Similarly, when η=arctan (acceleration in a horizontal direction at the time of depression/acceleration in a running direction at the time of depression) using the acceleration in a horizontal direction and the acceleration in a running direction at the time of depression, propulsion efficiency 2 in the horizontal direction can be calculated as the propulsion efficiency 2 in the horizontal direction=cos η×100(%). Alternatively, an ideal angle η′ can be calculated using data of a plurality of persons who fast run, and propulsion efficiency 2 in the horizontal direction can be calculated as propulsion efficiency 2 in a horizontal direction={1−|(η′−η)/η′|}×100(%).

Also, in addition, propulsion efficiency 2 in the vertical direction can be calculated by replacing ξ with arctan (speed in the vertical direction at the time of depression/speed in the running direction at the time of depression). Similarly, propulsion efficiency 2 in the horizontal direction can also be calculated by replacing η with arctan (speed in the horizontal direction at the time of depression/speed in the running direction at the time of depression).

### Propulsion Efficiency 3

Propulsion efficiency 3 is an exercise index indicating whether the kicking force efficiently becomes propulsion, using a jump angle. When a highest arrival point in a vertical direction in one step (½ of amplitude of a distance in the vertical direction) is H and a distance in the running direction from kicking to landing is X, propulsion efficiency 3 can be calculated using Equation (6).

$$Propulsion efficiency 3 = arcsin ( \sqrt{\frac{16 {H}^{2} }{{X}^{2} + 16 {H}^{2}}} ) \qquad ( 6 )$$

### Propulsion Efficiency 4

Propulsion efficiency 4 is an exercise index indicating whether the kicking force efficiently becomes propulsion power using a ratio of energy used to advance in the running direction to total energy generated in one step, and is calculated as propulsion efficiency 4=(energy used to advance in the running direction/energy used for one step)×100(%). This energy is a sum of positional energy and kinetic energy.

### Amount of Energy Consumption

An amount of energy consumption is an exercise index defined as an amount of energy consumed by one-step advance, and also indicates integration in the running period of an amount of energy consumed by one-step advance. The amount of energy consumption is calculated as an amount of energy consumption=(amount of energy consumption in the vertical direction+amount of energy consumption in the running direction+amount of energy consumption in the horizontal direction). Here, the amount of energy consumption in the vertical direction is calculated as amount of energy consumption in the vertical direction=(weight×gravity×distance in the vertical direction). Further, the amount of energy consumption in the running direction is calculated as amount of energy consumption in the running direction=[weight×{(maximum speed in the running direction after kicking)²−(minimum speed in the running direction after landing)²}/2]. Further, the amount of energy consumption in the horizontal direction is calculated by amount of energy consumption in the horizontal direction=[weight×{(maximum speed in the horizontal direction after kicking)²−(minimum speed in the horizontal direction after landing)²}/2].

### Landing Shock

The landing shock is an exercise index indicating how much shock is applied to a body due to landing, and is calculated by landing shock=(shock force in the vertical direction+shock force in the running direction+shock force in the horizontal direction). Here, the shock force in the vertical direction=(weight×speed in the vertical direction/shock time at the time of landing). Further, the shock force in the running direction={weight×(speed in the running direction before landing−minimum speed in the running direction after landing)/shock time}. Further, shock force in the horizontal direction={weight×(speed in the horizontal direction before landing−minimum speed in the horizontal direction after landing)/shock time}.

### Running Capability

Running capability is an exercise index of running power of the user. For example, a ratio of the stride and the ground time is known to have a correlation with the running record (time) (“About Ground Time and time of separation from ground During 100 m Race”, Journal of Research and Development for Future The exercises. 3(1): 1-4, 2004.), and is calculated using running capability=(stride/ground time).

### Anteversion Angle

An anteversion angle is an exercise index indicating how much the torso of the user is inclined with respect to the ground. The anteversion angle in a state in which the user stands perpendicular to the ground is 0, the anteversion angle when the user slouches is a positive value, and the anteversion angle when the user leans back is a negative value. The anteversion angle is obtained by converting the pitch angle of the m frame to be the specification as described above. When the exercise analysis device 2 (inertial measurement unit 10) is mounted on the user, there is a possibility that there is already a slope, and thus, a time of rest is assumed to be a 0 degree in the left figure, and the anteversion angle may be calculated using a resultant amount of change.

### Degree of Timing Matching

A degree of timing matching is an exercise index indicating how close the timing of the feature point of the user is to a good timing. For example, an exercise index indicating how close a timing of waist rotation is to a timing of kicking is considered. In a running way in which the leg is flowing, since one leg still remains behind the body when the other leg arrives, the running way in which the leg is flowing can be determined when the rotation timing of the waist comes after the kicking. When the waist rotation timing substantially matches the timing of the kicking, the running way is said to be good. On the other hand, when the waist rotation timing is later than the timing of the kicking, the running way is said to be a way in which the leg is flowing.

### Flow of Leg

A flow of a leg is an exercise index indicating a degree of the leg being backward at a time at which a kicking leg subsequently lands. The flow of the leg is calculated, for example, as an angle of a femur of a rear leg at the time of landing. For example, an index having a correlation with the flow of the leg is calculated. From this index, the angle of the femur of the rear leg at the time of landing can be estimated using a previously obtained correlation equation.

The index having a correlation with the flow of the leg is calculated, for example, as (time when the waist is rotated to the maximum in the yaw direction−time at the time of landing). The “time when the waist is rotated to the maximum in the yaw direction” is the time of start of an operation of the next step. When a time from the landing to the next operation is long, it takes time to pull back the leg, and a phenomenon in which the leg is flowing occurs.

Alternatively, the index having a correlation with the flow of the leg is calculated as (yaw angle when the waist is rotated to the maximum in the yaw direction−yaw angle at the time of landing). When a change in the yaw angle from the landing to the next operation is large, there is an operation to pull back the leg after landing, and this appears as a change in the yaw angle. Therefore, a phenomenon in which the leg is flowing occurs.

Alternatively, the pitch angle at the time of landing may be the index having a correlation with the flow of the leg. When the leg is high backward, a body (waist) is tilted forward. Therefore, the pitch angle of the sensor attached to the waist increases. When the pitch angle is large at the time of landing, a phenomenon in which the leg is flowing occurs.

### 1-3-7. Second Analysis Information

Hereinafter, each item of the second analysis information calculated by the second analysis information generation unit 276 will be described in detail.

### Energy Loss

An energy loss is an exercise index indicating an amount of energy wasted in an amount of energy consumed by one-step advance, and also indicates integration in a running period of an amount of energy wasted in the amount of energy consumed by one-step advance. The energy loss is calculated by energy loss={amount of energy consumption×(100−directly-under landing rate)×(100−propulsion efficiency)}. Here, the directly-under landing rate is any one of directly-under landing rates 1 to 3, and the propulsion efficiency is any one of propulsion efficiencies 1 to 4.

### Energy Efficiency

Energy efficiency is an exercise index indicating whether the energy consumed by one-step advance is effectively used as energy for advance in the running direction, and also indicates integration in the running period. Energy efficiency is calculated as energy efficiency={(amount of energy consumption−energy consumption loss)/amount of energy consumption}.

### Load on the Body

A load on the body is an exercise index indicating how much shock is applied to the body through accumulation landing shock. Since injury is caused due to the accumulation of the shock, ease of injury can be determined by evaluating the load on the body. The load on the body is calculated by the load on the body=(load on a right leg+load on a left leg). The load on the right leg can be calculated by integrating landing shock of the right leg. The load on the left leg can be calculated by integrating landing shock of the left leg. Here, for the integration, both integration during running and integration from the past can be performed.

### 1-3-8. Left-Right Difference Ratio (Left-Right Balance)

A left-right difference ratio is an exercise index indicating how much the left and right of the body are different from each other for the running pitch, the stride, the ground time, the shock time, each item of the first analysis information, and each item of the second analysis information, and is assumed to indicate how much the left leg is different from the right leg. The left-right difference ratio is calculated as left-right difference ratio=(numerical value of left leg/numerical value of right leg×100) (%), and the numerical value is each numerical value of the running pitch, the stride, the ground time, the shock time, the amount of brake, the propulsion power, the directly-under landing rate, the propulsion efficiency, the speed, the acceleration, the running distance, the anteversion angle, the flow of a leg, the rotation angle of the waist, the rotation angular speed of the waist, the amount of inclination to left and right, the shock time, the running capability, the amount of energy consumption, the energy loss, the energy efficiency, the landing shock, and the load on the body. Further, the left-right difference ratio also includes an average value or a dispersion of each numerical value.

### 1-3-9. Procedure of the Process

FIG. 14 is a flowchart diagram illustrating an example of a procedure of the exercise analysis process performed by the processing unit 20. The processing unit 20 executes the exercise analysis program 300 stored in the storage unit 30 to execute the exercise analysis process, for example, in the procedure of the flowchart of FIG. 14.

As illustrated in FIG. 14, the processing unit 20 waits until the processing unit 20 receives a measurement start command (N in S10). When the processing unit 20 receives the measurement start command (Y in S10), the processing unit 20 first calculates an initial posture, an initial position, and an initial bias using the sensing data measured by the inertial measurement unit 10, and the GPS data on the assumption that the user is at rest (S20).

Then, the processing unit 20 acquires the sensing data from the inertial measurement unit 10, and adds the acquired sensing data to the sensing data table 310 (S30).

Then, the processing unit 20 performs the inertial navigation operation process to generate operation data including various information (S40). An example of a procedure of this inertial navigation operation process will be described below.

Then, the processing unit 20 performs the exercise analysis information generation process using the calculation data generated in S40 to generate exercise analysis information (S50). An example of a procedure of this exercise analysis information generation process will be described below.

Then, the processing unit 20 generates the output information during running using the exercise analysis information generated in S40 and transmits the output information during running to the reporting device 3 (S60).

Also, the processing unit 20 repeats the process of S30 and subsequent steps each time the sampling period Δt elapses after the processing unit 20 acquires previous sensing data (Y in S70) until the processing unit 20 receives the measurement end command (N in S70 and N in S80).

When the processing unit 20 receives the measurement end command (Y in S80), the processing unit 20 generates the running result information using the exercise analysis information generated in S50, transmits the running result information to the reporting device 3 (S90), and ends the exercise analysis process.

FIG. 15 is a flowchart diagram illustrating an example of a procedure of the inertial navigation operation process (process of S40 in FIG. 14). The processing unit 20 (inertial navigation operation unit 22) executes the inertial navigation operation program 302 stored in the storage unit 30, for example, to execute the inertial navigation operation process in the procedure of the flowchart of FIG. 15.

As illustrated in FIG. 15, first, the processing unit 20 removes the bias from the acceleration and the angular speed included in the sensing data acquired in S30 of FIG. 14 using the initial bias calculated in S20 in FIG. 14 (using the acceleration bias bₐ and the angular speed bias b_{ω} after the acceleration bias bₐ and the angular speed bias b_{ω} are estimated in S150 to be described below) to correct the acceleration and the angular speed, and updates the sensing data table 310 with the corrected acceleration and angular speed (S100).

The processing unit 20 then integrates the sensing data corrected in S100 to calculate a speed, a position, and a posture angle, and adds calculation data including the calculated speed, position, and posture angle to the operation data table 340 (S110).

The processing unit 20 then performs a running detection process (S120). An example of a procedure of this running detection process will be described below.

Then, when the processing unit 20 detects a running period through the running detection process (S120) (Y in S130), the processing unit 20 calculates a running pitch and a stride (S140). Further, when the processing unit 20 does not detect the running period (N in S130), the processing unit 20 does not perform the process of S140.

Then, the processing unit 20 performs an error estimation process to estimate the speed error δv^{e}, the posture angle error εₑ, the acceleration bias bₐ, the angular speed bias b_{ω}, and the position error δp^{e} (S150).

The processing unit 20 then corrects the speed, the position, and the posture angle using the speed error δv^{e}, the posture angle error ε^{e}, and position error δp^{e} estimated in S150, respectively, and updates the operation data table 340 with the corrected speed, position, and posture angle (S160). Further, the processing unit 20 integrates the speed corrected in S160 to calculate a distance of the e frame (S170).

The processing unit 20 then coordinate-transforms the sensing data (acceleration and angular speed of the b frame) stored in the sensing data table 310, the calculation data (the speed, the position, and the posture angle of the e frame) stored in the operation data table 340, and the distance of the e frame calculated in S170 into acceleration, angular speed, speed, position, posture angle, and distance of the m frame (S180).

Also, the processing unit 20 generates operation data including the acceleration, angular speed, speed, position, posture angle, and distance of the m frame after the coordinate transformation in S180, and the stride and the running pitch calculated in S140 (S190). The processing unit 20 performs the inertial navigation operation process (process of S100 to S190) each time the processing unit 20 acquires the sensing data in S30 of FIG. 14.

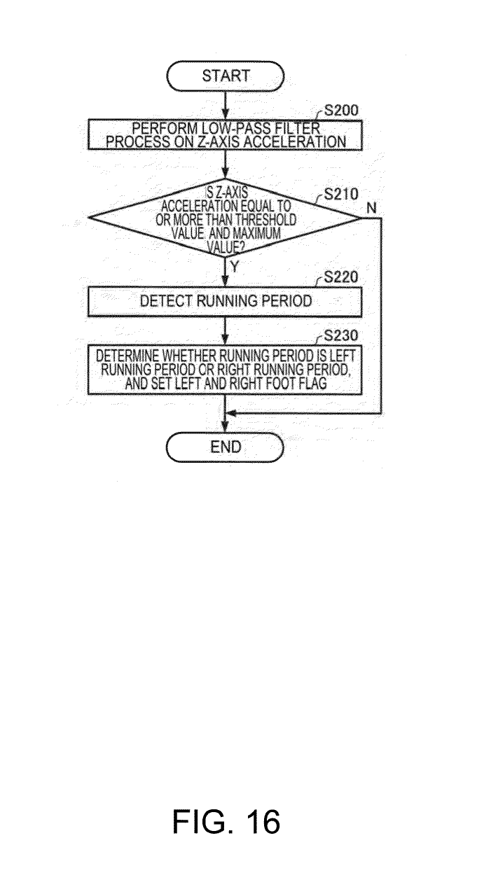

FIG. 16 is a flowchart diagram illustrating an example of a procedure of the running detection process (S120 in FIG. 15). The processing unit 20 (the running detection unit 242) executes the running detection process, for example, in the procedure of the flowchart of FIG. 16.

As illustrated in FIG. 16, the processing unit 20 performs a low-pass filter process on the z-axis acceleration included in the acceleration corrected in S100 of FIG. 15 (S200) to remove noise.

Then, when the z-axis acceleration subjected to the low-pass filter process in S200 is equal to or more than a threshold value and is a maximum value (Y in S210), the processing unit 20 detects a running period at this timing (S220).

The processing unit 20 then determines whether the running period detected in S220 is a left running period or a right running period, sets the left and right foot flag (S230), and ends the running detection process. When the z-axis acceleration is smaller than the threshold value and is not the maximum value (N in S210), the processing unit 20 ends the running detection process without performing the process of S220 and subsequent steps.

FIG. 17 is a flowchart diagram illustrating an example of a procedure of the exercise analysis information generation process (process in S50 of FIG. 14) in the first embodiment. The processing unit 20 (exercise analysis unit 24) executes the exercise analysis information generation program 304 stored in the storage unit 30 to execute the exercise analysis information generation process, for example, in the procedure of the flowchart of FIG. 17.

As illustrated in FIG. 17, first, the processing unit 20 calculates respective items of the basic information using the operation data generated through the inertial navigation operation process in S40 of FIG. 14 (S300).

The processing unit 20 then performs a process of detecting the feature point (for example, landing, depression, or separation from ground) in the running operation of the user using the operation data (S310).

When the processing unit 20 detects the feature point in the process of S310 (Y in S320), the processing unit 20 calculates the ground time and the shock time based on a timing of detection of the feature point (S330). Further, the processing unit 20 uses a part of the operation data and the ground time and the shock time generated in S330 as input information, and calculates some items of the first analysis information (item requiring information on the feature point for calculation) based on the timing of detection of the feature point (S340). When the processing unit 20 does not detect the feature point in the process of S310 (N in S320), the processing unit 20 does not perform the process of S330 and S340.

The processing unit 20 then calculates other items (items not requiring the information on the feature point for calculation) of the first analysis information using the input information (S350).

The processing unit 20 then calculates respective items of the second analysis information using the first analysis information (S360).

The processing unit 20 then calculates the left-right difference ratio for each item of the input information, each item of the first analysis information, and each item of the second analysis information (S370).

The processing unit 20 adds a current measurement time to respective information calculated in S300 to S370, stores the resultant information in the storage unit 30 (S380), and ends the exercise analysis information generation process.

### 1-4. Reporting Device

### 1-4-1. Configuration of the Reporting Device

FIG. 18 is a functional block diagram illustrating an example of a configuration of the reporting device 3. As illustrated in FIG. 18, the reporting device 3 includes a processing unit 120, a storage unit 130, a communication unit 140, a manipulation unit 150, a clocking unit 160, a display unit 170, a sound output unit 180, and a vibration unit 190. However, in the reporting device 3 of the present embodiment, some of these components may be removed or changed, or other components may be added.

The storage unit 130 includes, for example, a recording medium that stores a program or data, such as a ROM, a flash ROM, a hard disk, or a memory card, or a RAM that is a work area of the processing unit 120.

The communication unit 140 is a communication unit that performs data communication with the communication unit 40 of the exercise analysis device 2 (see FIG. 3) or the communication unit 440 of the information analysis device 4 (see FIG. 21), and performs, for example, a process of receiving a command (for example, measurement start/measurement end command) according to manipulation data from the processing unit 120 and transmitting the command to the communication unit 40 of the exercise analysis device 2, a process of receiving the output information during running or the running result information transmitted from the communication unit 40 of the exercise analysis device 2 and sending the information to the processing unit 120, or a process of receiving information on the target value of each exercise index transmitted from the communication unit 440 of the information analysis device 4 and sending the information to the processing unit 120.

The manipulation unit 150 performs a process of acquiring the manipulation data (for example, manipulation data for measurement start/measurement end, or manipulation data for selection of display content) from the user, and sending the manipulation data to the processing unit 120. The manipulation unit 150 may be, for example, a touch panel display, a button, a key, or a microphone.

The clocking unit 160 performs a process of generating time information such as year, month, day, hour, minute, and second. The clocking unit 160 is implemented by, for example, a real time clock (RTC) IC, or the like.

The display unit 170 displays image data or text data sent from the processing unit 120 as a character, a graph, a table, an animation, or other images. The display unit 170 is implemented by, for example, a display such as a liquid crystal display (LCD), an organic electroluminescence (EL) display, or an electrophoretic display (EPD), and may be a touch panel display. Also, functions of the manipulation unit 150 and the display unit 170 may be implemented by one touch panel display.

The sound output unit 180 outputs sound data sent from the processing unit 120 as sound such as voice or buzzer sound. The sound output unit 180 is implemented by, for example, a speaker or a buzzer.

The vibration unit 190 vibrates according to vibration data sent from the processing unit 120. This vibration can be delivered to the reporting device 3, and the user with the reporting device 3 can feel the vibration. The vibration unit 190 is implemented by, for example, a vibration motor.

The processing unit 120 includes, for example, a CPU, a DSP, and an ASIC, and executes a program stored in the storage unit 130 (recording medium) to perform various operation processes or control processes. For example, the processing unit 120 performs various processes according to the manipulation data received from the manipulation unit 150 (for example, a process of sending a measurement start/measurement end command to the communication unit 140, or a display process or a sound output process according to the manipulation data), a process of receiving the output information during running from the communication unit 140, generating text data or image data according to the exercise analysis information, and sending the data to the display unit 170, a process of generating sound data according to the exercise analysis information and sending the sound data to the sound output unit 180, and a process of generating vibration data according to the exercise analysis information and sending the vibration data to the vibration unit 190. Further, the processing unit 120 performs, for example, a process of generating time image data according to the time information received from the clocking unit 160 and sending the time image data to the display unit 170.

Further, in the present embodiment, the processing unit 120, for example, acquires information on target values of various exercise indexes transmitted from the information analysis device 4 via the communication unit 140 prior to running of the user (prior to transmission of the measurement start command), and performs setup. Further, the processing unit 120 may set the target value for each exercise index based on the manipulation data received from the manipulation unit 150. Also, the processing unit 120 compares the value of each exercise index included in the output information during running with each target value, generates information on the exercise state in the running of the user according to a comparison result, and reports the information to the user via the sound output unit 180 or the vibration unit 190.

For example, by manipulating the information analysis device 4 or the manipulation unit 150, the user may set the target value based on the value of each exercise index in past running of the user, may set the target value based on, for example, an average value of each exercise index of another member belonging to the same running team, may set a value of each exercise index of a desired runner or a target runner to the target value, or may set a value of each exercise index of another user who clears the target time to the target value.

The exercise index to be compared with the target value may be all exercise indexes included in the output information during running, or may be only a specific exercise index that is determined in advance, and the user may manipulate the manipulation unit 150 or the like to select the exercise index.

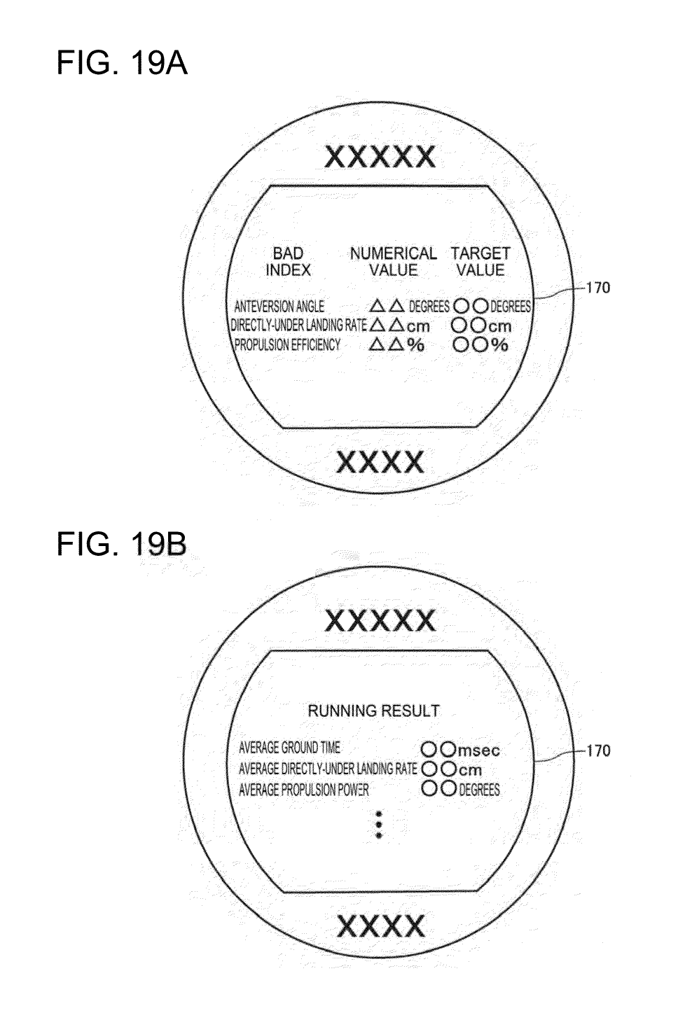

For example, when there is a worse exercise index than the target value, the processing unit 120 reports the worse exercise index through sound or vibration, and displays the value of the worse exercise index than the target value on the display unit 170. The processing unit 120 may generate a different type of sound or vibration according to a type of worse exercise index than the target value, or may change the type of sound or vibration according to a degree of being worse than the target value for each exercise index. When there are a plurality of worse exercise indexes than the target values, the processing unit 120 may generate sound or vibration of the type according to the worst exercise index and may display information on the values of all the worse exercise indexes than the target values, and the target values on the display unit 170, for example, as illustrated in FIG. 19A.

The user can continue to run while recognizing which exercise index is worst and how much the exercise index is worse from a type of sound or vibration without viewing the information displayed on the display unit 170. Further, the user can accurately recognize a difference between the values of all worse exercise indexes than the target values and the target values when viewing the information displayed on the display unit 170.

Further, the exercise index that is a target for which sound or vibration is generated may be selected from among the exercise indexes to be compared with target values by the user manipulating the manipulation unit 150 or the like. In this case, for example, information on the values of all the worse exercise indexes than the target values, and the target values may be displayed on the display unit 170.

Further, the user may perform setup of a reporting period (for example, setup such as generation of sound or vibration for 5 seconds every one minute) through the manipulation unit 150, and the processing unit 120 may perform reporting to the user according to the set reporting period.

Further, in the present embodiment, the processing unit 120 acquires the running result information transmitted from the exercise analysis device 2 via the communication unit 140, and displays the running result information on the display unit 170. For example, as illustrated in FIG. 19B, the processing unit 120 displays an average value of each exercise index during running of the user, which is included in the running result information, on the display unit 170. When the user views the display unit 170 after the running end (after the measurement end manipulation), the user can immediately recognize the goodness or badness of each exercise index.

### 1-4-2. Procedure of the Process

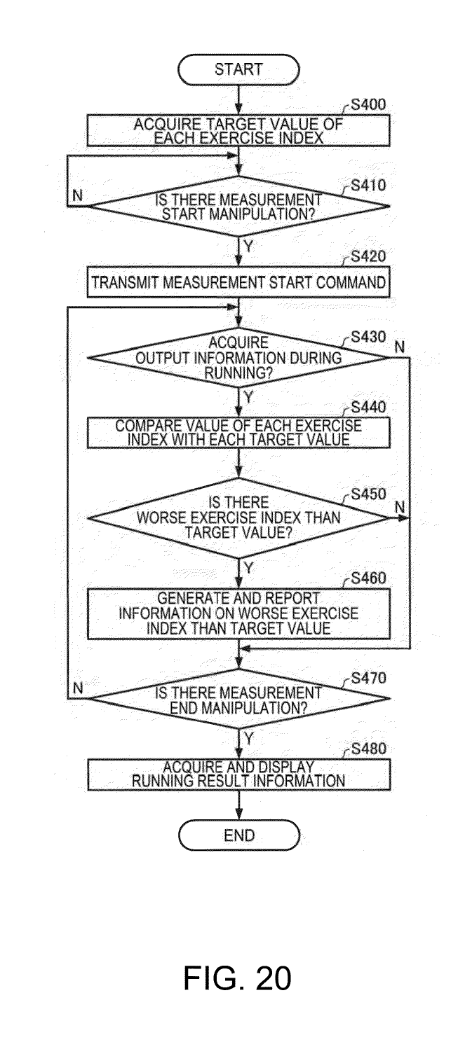

FIG. 20 is a flowchart diagram illustrating an example of a procedure of a reporting process performed by the processing unit 120 in the first embodiment. The processing unit 120 executes the program stored in the storage unit 130, for example, to execute the reporting process in the procedure of the flowchart of FIG. 20.

As illustrated in FIG. 20, the processing unit 120 first acquires the target value of each exercise index from the information analysis unit 4 via the communication unit 140 (S400).

Then, the processing unit 120 waits until the processing unit 120 acquires the manipulation data of measurement start from the manipulation unit 150 (N in S410). When the processing unit 120 acquires the manipulation data of measurement start (Y in S410), the processing unit 120 transmits the measurement start command to the exercise analysis device 2 via the communication unit 140 (S420).

Then, the processing unit 120 compares the value of each exercise index included in the acquired output information during running with each target value acquired in S400 (S440) each time the processing unit 120 acquires the output information during running from the exercise analysis device 2 via the communication unit 140 (Y in S430) until the processing unit 120 acquires the manipulation data of the measurement end from the manipulation unit 150 (N in S470).

When there is a worse exercise index than the target value (Y in S450), the processing unit 120 generates information on the worse exercise index than the target value and reports the information to the user using sound, vibration, text, or the like via the sound output unit 180, the vibration unit 190, and the display unit 170 (S460).

On the other hand, when there is no worse exercise index than the target value (N in S450), the processing unit 120 does not perform the process of S460.

Also, when the processing unit 120 acquires the manipulation data of the measurement end from the manipulation unit 150 (Y in S470), the processing unit 120 acquires the running result information from the exercise analysis device 2 via the communication unit 140, displays the running result information on the display unit 170 (S480), and ends the reporting process.

Thus, the user can run while recognizing the running state based on the information reported in S450. Further, the user can immediately recognize the running result after running end, based on the information displayed in S480.

### 1-5. Information Analysis Device

### 1-5-1. Configuration of the Information Analysis Device

FIG. 21 is a functional block diagram illustrating an example of a configuration of the information analysis device 4. As illustrated in FIG. 21, the information analysis device 4 includes a processing unit 420, a storage unit 430, a communication unit 440, a manipulation unit 450, a communication unit 460, a display unit 470, and a sound output unit 480. However, in the information analysis device 4 of the present embodiment, some of these components may be removed or changed, or other components may be added.

The communication unit 440 is a communication unit that performs data communication with the communication unit 40 of the exercise analysis device 2 (see FIG. 3) or the communication unit 140 of the reporting device 3 (see FIG. 18). The communication unit 440 performs, for example, a process of receiving the transmission request command for requesting transmission of the exercise analysis information designated according to the manipulation data (exercise analysis information included in the running data that is a registration target) from the processing unit 420, transmitting the transmission request command to the communication unit 40 of the exercise analysis device 2, receiving the exercise analysis information from the communication unit 40 of the exercise analysis device 2, and sending the exercise analysis information to the processing unit 420, and a process of receiving the information on the target value of each exercise index from the processing unit 420 and transmitting the information to the communication unit 140 of the reporting device 3.

The communication unit 460 is a communication unit that performs data communication with the server 5, and performs, for example, a process of receiving running data that is a registration target from the processing unit 420 and transmitting the running data to the server 5 (running data registration process), and a process of receiving management information corresponding to manipulation data of registration, editing, and deletion of a user, registration, editing, and deletion of a group, and editing, deletion, and replacement of the running data from the processing unit 420 and transmitting the management information to the server 5.

The manipulation unit 450 performs a process of acquiring manipulation data from the user (manipulation data of registration, editing, and deletion of the user, registration, editing, and deletion of a group, and editing, deletion, and replacement of the running data, manipulation data for selecting the user that is an analysis target, or manipulation data for setting a target value of each exercise index), and sending the manipulation data to processing unit 420. The manipulation unit 450 may be, for example, a touch panel display, a button, a key, or a microphone.

The display unit 470 displays image data or text data sent from the processing unit 420 as a text, a graph, a table, animation, or other images. The display unit 470 is implemented by, for example, a display such as an LCD, an organic EL display, or an EPD, and may be a touch panel display. Also, functions of the manipulation unit 450 and the display unit 470 may be implemented by one touch panel display.

The sound output unit 480 outputs sound data sent from the processing unit 420 as sound such as voice or buzzer sound. The sound output unit 480 is implemented by, for example, a speaker or a buzzer.

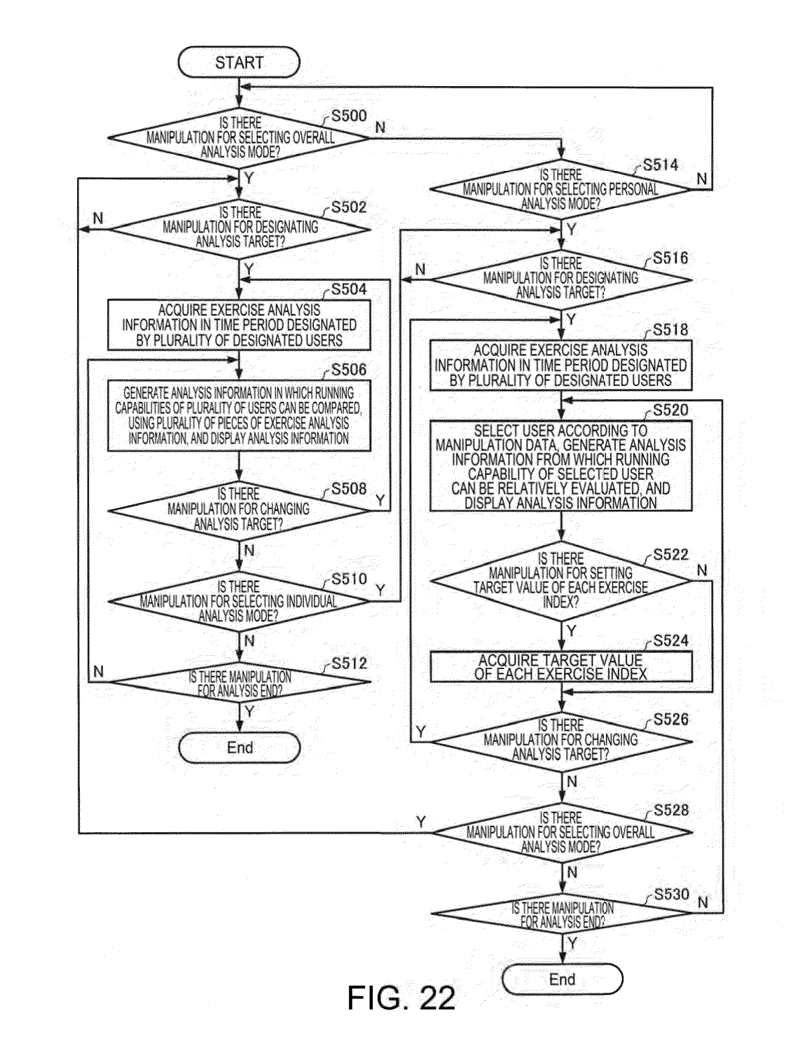

The storage unit 430 includes, for example, a recording medium that stores a program or data, such as a ROM, a flash ROM, a hard disk, or a memory card, or a RAM that is a work area of the processing unit 420. An analysis program 432 read by the processing unit 420, for executing the analysis process (see FIG. 22) is stored in the storage unit 430 (one of the recording media).

The processing unit 420 includes, for example, a CPU, a DSP, and an ASIC, and executes various programs stored in the storage unit 430 (recording medium) to perform various operation processes or control processes. For example, the processing unit 420 performs a process of transmitting a transmission request command for requesting transmission of the exercise analysis information designated according to the manipulation data received from the manipulation unit 450 to the exercise analysis device 2 via the communication unit 440, and receiving the exercise analysis information from the exercise analysis device 2 via the communication unit 440, or a process of generating running data (running data that is registration data) including the exercise analysis information received from the exercise analysis device 2 according to the manipulation data received from the manipulation unit 450, and transmitting the running data to the server 5 via the communication unit 460. Further, the processing unit 420 performs a process of transmitting management information according to the manipulation data received from the manipulation unit 450 to the server 5 via the communication unit 460. The processing unit 420 performs a process of transmitting a transmission request for the running data that is an analysis target selected according to the manipulation data received from the manipulation unit 450 to the server 5 via the communication unit 460, and receiving the running data that is an analysis target from the server 5 via the communication unit 460. Further, the processing unit 420 performs a process of analyzing the running data of a plurality of users that are analysis targets selected according to the manipulation data received from the manipulation unit 450 to generate analysis information that is information on the analysis result, and sending the analysis information to the display unit 470 or the sound output unit 480, for example, as text data or image data, and sound data. Further, the processing unit 420 performs a process of storing the target value of each exercise index set according to the manipulation data received from the manipulation unit 450 in the storage unit 430, or a process of reading the target value of each exercise index from the storage unit 430 and transmitting the target value to the reporting device 3.

In particular, in the present embodiment, the processing unit 420 executes the analysis program 432 stored in the storage unit 430 to function as an exercise analysis information acquisition unit 422, an analysis information generation unit 424, and a target value acquisition unit 426. However, the processing unit 420 may receive and execute the analysis program 432 stored in any storage device (recording medium) via a network or the like.

The exercise analysis information acquisition unit 422 performs a process of acquiring a plurality of pieces of exercise analysis information that are the information on the analysis results of the exercises of the plurality of users that are analysis targets from the database of the server 5 (or the exercise analysis device 2). The plurality of pieces of exercise analysis information acquired by the exercise analysis information acquisition unit 422 are stored in the storage unit 430. Each of the plurality of pieces of exercise analysis information may be generated by the same exercise analysis device 2 or may be generated by any one of a plurality of different exercise analysis devices 2. In the present embodiment, each of the plurality of pieces of exercise analysis information acquired by the exercise analysis information acquisition unit 422 includes the values of various exercise indexes of each of the plurality of users (for example, various exercise indexes described above).

The analysis information generation unit 424 performs a process of generating analysis information from which the running capabilities of a plurality of users that are analysis targets can be compared, using the plurality of pieces of exercise analysis information acquired by the exercise analysis information acquisition unit 422. The analysis information generation unit 424, for example, may generate the analysis information using the exercise analysis information of a plurality of users that are analysis targets selected in the manipulation data received from the manipulation unit 450 or may generate analysis information using the exercise analysis information of the plurality of users that are analysis targets in a time period selected in the manipulation data received from the manipulation unit 450.

In the present embodiment, the analysis information generation unit 424 selects any one of an overall analysis mode and a personal analysis mode according to the manipulation data received from the manipulation unit 450, and generates analysis information from which running capability of a plurality of users can be compared in each selected analysis mode.

The analysis information generation unit 424 may generate analysis information from which the running capabilities of a plurality of users that are analysis targets can be compared, on each date on which the plurality of users run in the overall analysis mode. For example, when five users run three times on July 1, July 8, and July 15, the analysis information generation unit 424 may generate analysis information from which the running capabilities of five users on July 1, July 8, and July 15 can be compared.

Further, the plurality of users that are analysis targets are classified into a plurality of groups, and the analysis information generation unit 424 may generate analysis information from which the running capabilities of the plurality of users can be compared for each group in the overall analysis mode. For example, when for five users 1 to 5, users 1, 3 and 5 are classified into group 1, and users 2 and 4 are classified into group 2, the analysis information generation unit 424 may generate analysis information from which the running capabilities of three users 1, 3 and 5 belonging to group 1 can be compared or analysis information from which the running capabilities of two users 2 and 4 belonging to group 2 can be compared.

Further, the analysis information generation unit 424 may generate analysis information from which running capability of an arbitrary user (an example of a first user) included in the plurality of users can be relatively evaluated, using the values of the exercise indexes of the plurality of users that are analysis targets in the personal analysis mode. The arbitrary user may be, for example, a user selected in the manipulation data received from the manipulation unit 450. For example, the analysis information generation unit 424 may set the highest index value among the exercise index values of the plurality of users that are analysis targets to 10 and the lowest index value to 0, converts the exercise index value of the arbitrary user into a value of 0 to 10, and generate analysis information including information on the converted exercise index value, or may calculate a deviation value of the exercise index value for the arbitrary user using the exercise index values of the plurality of users that are analysis targets and generate analysis information including information on the deviation value.

The target value acquisition unit 426 performs a process of acquiring target values of the various exercise indexes of an arbitrary user (for example, a user selected in the manipulation data) included in the plurality of users that are analysis targets. This target value is stored in the storage unit 430, and the analysis information generation unit 424 generates analysis information from which values of various exercise indexes of the arbitrary user and the respective target values can be compared, using the information stored in the storage unit 430 in the personal analysis mode.

The processing unit 420 generates display data such as a text or an image or sound data such as voice using the analysis information generated by the analysis information generation unit 424, and outputs the data to the display unit 470 or the sound output unit 480. Thus, an analysis result of the plurality of users that are analysis targets is present from the display unit 470 or the sound output unit 480.

Further, the processing unit 420 performs a process of transmitting the target value of each exercise index of the user acquired by the target value acquisition unit 426 and stored in the storage unit 430, to the reporting device 3 through the communication unit 440 before the user wears the exercise analysis device 2 and runs. As described above, the reporting device 3 receives the target value of each exercise index, receives the value of each exercise index (which is included in the output information during running) from the exercise analysis device 2, compares the value of each exercise index with each target value, and reports information on the exercise state of the user during running according to a comparison result through sound or vibration (and through a text or an image).

### 1-5-2. Procedure of the Process

FIG. 22 is a flowchart diagram illustrating an example of a procedure of the analysis process performed by the processing unit 420 of the information analysis device 4. The processing unit 420 of the information analysis device 4 (an example of a computer) executes the analysis program 432 stored in the storage unit 430 to execute, for example, the analysis process in the procedure of the flowchart in FIG. 22.

First, the processing unit 420 waits until the processing unit 420 acquires manipulation data for selecting an overall analysis mode or manipulation data for selecting a personal analysis mode (N in S500 of N and S514).

When the processing unit 420 acquires the manipulation data for selecting the overall analysis mode (Y in S500), the processing unit 420 waits until the processing unit 420 acquires manipulation data for designating an analysis target (N in S502). When the processing unit 420 acquires the manipulation data for designating an analysis target (Y in S502), the processing unit 420 acquires the exercise analysis information (specifically, the running data) in a time period designated by a plurality of users designated in the manipulation data, from a database of the server 5 via the communication unit 460, and stores the exercise analysis information in the storage unit 430 (S504).

Then, the processing unit 420 generates analysis information in which running capabilities of a plurality of users that are analysis targets can be compared, using a plurality of pieces of exercise analysis information (running data) acquired in S504, and displays the analysis information on the display unit 270 (S506).

Then, unless the processing unit 420 acquires any one of manipulation data for changing the analysis target, manipulation data for selecting the personal analysis mode, and manipulation data for analysis end (N in S508, N in S510, and N in S512), the processing unit 420 performs process of S506.

When the processing unit 420 acquires the manipulation data for changing the analysis target (Y in S508), the processing unit 420 performs the processes of S504 and S506 again. When the processing unit 420 acquires the manipulation data for the analysis end (Y in S512), the processing unit 420 end the analysis process.

Further, when the processing unit 420 acquires the manipulation data for selecting the personal analysis mode (Y in S510 or Y in S514), the processing unit 420 waits until the processing unit 420 acquires manipulation data for designating the analysis target (N in S516). When the processing unit 420 acquires the manipulation data for designating the analysis target (Y in S516), the processing unit 420 acquires the exercise analysis information (specifically, running data) in the time period designated by the plurality of users designated in the manipulation data from the database of the server 5 via the communication unit 460, and stores the exercise analysis information in the storage unit 430 (S518).

Then, the processing unit 420 selects a user according to the manipulation data acquired from the manipulation unit 450, generates analysis information from which running capability of the selected user can be relatively evaluated using the plurality of pieces of exercise analysis information acquired in S518, and displays the analysis information on the display unit 470 (S520).

Then, when the processing unit 420 acquires manipulation data for setting a target value of each exercise index for the user selected in S520 (Y in S522), the processing unit 420 acquires the target value of each exercise index set in the manipulation data, and stores the target value in the storage unit 430 (S524).

Then, unless the processing unit 420 acquires any one of the manipulation data for changing the analysis target, the manipulation data for selecting the overall analysis mode, and the manipulation data for the analysis end (N in S526, N in S528, and N in S530), the processing unit 420 performs the process of S520.

When the processing unit 420 acquires the manipulation data for changing the analysis target (Y in S526), the processing unit 420 performs the processes of S518 and S520 again. When the processing unit 420 acquires the manipulation data for the analysis end (Y in S530), the processing unit 420 ends the analysis process.

Further, when the processing unit 420 acquires the manipulation data for selecting the overall analysis mode (Y in S528), the processing unit 420 performs the process of S502 and subsequent steps again.

### 1-5-3. Specific Example of the Analysis Process

Hereinafter, an analysis process in the processing unit 420 will be specifically described using, as an example, an application with which a manager such as a supervisor or a coach can manage and analyze running of a plurality of players belonging to a team (an example of the “plurality of users” described above), and each player can analyze the running of the player. FIGS. 23 to 33 are diagrams illustrating examples of screens displayed on the display unit 470 by the processing unit 20 executing the analysis program 432 that implements the application. In this example, five tab screens of “Management”, “Record”, “Player capability”, “Personal details”, and “Exercise diary” can be selected. FIG. 23 is a diagram illustrating an example of the management tab screen. As illustrated in FIG. 23, the management tab screen 500 includes three links for player management respectively displayed as “Register player”, “Edit player”, and “Delete player”, three links for group management respectively displayed as “Register group”, “Edit group”, and “Delete group”, four links for running data management respectively displayed as “Register data”, “Edit data”, “Delete data”, and “Replace data”, a link for management password change displayed as “Change password”, and a button for ending the analysis displayed as “End”. The manager can perform a variety of manipulations on the management tab screen 500 after inputting a pre-registered password.

When the manager selects link “Register player”, the processing unit 420 displays an input screen for face photo, name, date of birth, height, weight, and sex. When the manager inputs information on the player from the input screen, the processing unit 420 transmits the input information to the server 5, and the information on the player is registered in the database as information on a member of the team.

When the manager selects link “Edit player”, the processing unit 420 displays a selection screen for the name of the player. When the manager selects the name of the player, the processing unit 420 displays an editing screen including information such as the registered face photo, name, date of birth, height, weight, and sex of the selected player. When the manager modifies the information on the player from the editing screen, the processing unit 420 transmits the modified information to the server 5, and the registered information of the player is corrected.

When the manager selects link “Delete player”, the processing unit 420 displays the selection screen for the name of the player. When the manager selects the name of the player, the processing unit 420 transmits information on the selected name of the player to the server 5, and the registered information of the player is deleted.

When the manager selects link “Register group”, the processing unit 420 displays an input screen for a group name. When the manager inputs the group name from the input screen, the processing unit 420 displays a list of registered names of players. When the manager selects the name of the player from the list, the processing unit 420 transmits information on the input group name and the selected name of the player to the server 5, and all of the selected players are registered in the selected group. Also, each player can belong to a plurality of groups. For example, when there are seven groups: “freshman”, “sophomore”, “junior”, “senior”, “major league”, “minor league”, “third league”, each player can belong to one of the groups “freshman”, “sophomore”, “junior”, “senior”, and can belong to one of the groups “major league”, “minor league”, and “third league”.

When the manager selects link “Edit group”, the processing unit 420 displays a selection screen for the group name. When the manager selects the group name, the processing unit 420 displays a list of names of players not belonging to the selected group and a list of names of players belonging to the group. When the manager selects the name of the player from one of the lists and move to the name the other list, the processing unit 420 transmits information on the selected group name, the moved name of the player, and a movement direction (whether the name is added to the group or deleted from the group) to the server 5, and updates the player to be registered to the selected group.

When the manager selects link “Delete group”, the processing unit 420 displays the selection screen for the group name. When the manager selects the group name, the processing unit 420 transmits information on the selected group name to the server 5, and information on the registered group (association of registered players) is deleted.

When the manager selects the link “Register running data”, the processing unit 420 displays the selection screen for the file name of the exercise analysis information. When the manager selects the file name of the exercise analysis information from the selection screen, the processing unit 420 displays an input screen including, for example, a display column in which, for example, the file name of the selected exercise analysis information (running data name), running date included in the exercise analysis information, a name of the player, a distance, and time are automatically displayed, an input column for a course name, weather, temperature, and a remark, and a check box of an official meet (race). The remark input column is provided, for example, for input of exercise content or interest. When the manager inputs respective information of the input columns from the input screen and edits some pieces of the information (for example, distance or time) of the display column, if necessary, the processing unit 420 acquires the selected exercise analysis information from the exercise analysis device 2, and transmits running data including the exercise analysis information, each piece of information of the display column of the input screen, each piece of information of the input column, and information on ON/OFF of the check box to the server 5. The running data is registered in the database.

The processing unit 420 displays a selection screen for the name of the player and the running data name when the manager selects the link “Edit running data”. When the manager selects the name of the player and the running data name, the processing unit 420 displays an editing screen including, for example, a display column for the selected running data name of the running data, running date, the name of the player, a course name, a distance, a time, weather, a temperature, and a remark, and a check box of an official meet (race). When the manager edits any one of the course name, the distance, the time, the weather, the temperature, the remark, and the check box from the editing screen, the processing unit 420 transmits the modified information to the server 5, and information of the registered running data is modified.

When the manager selects the link “Delete running data”, the processing unit 420 displays a selection screen for the running data name. When the manager selects the running data name, the processing unit 420 transmits information on the selected running data name to the server 5, and the registered running data is deleted.

When the manager selects the link “Replace running data” link, the processing unit 420 displays a replacement screen for running data. When the manager selects a running data name to be replaced, the processing unit 420 transmits information on the running data name to be replaced to the server 5, and registered running data is overwritten with the running data after replacement.

When the manager selects the link “Change password”, the processing unit 420 displays an input screen for an old password and a new password. When the manager inputs an old password and a new password, the processing unit 420 transmits information on the input old password and the input new password to the server 5. When the old password matches the registered password, the new password is updated.

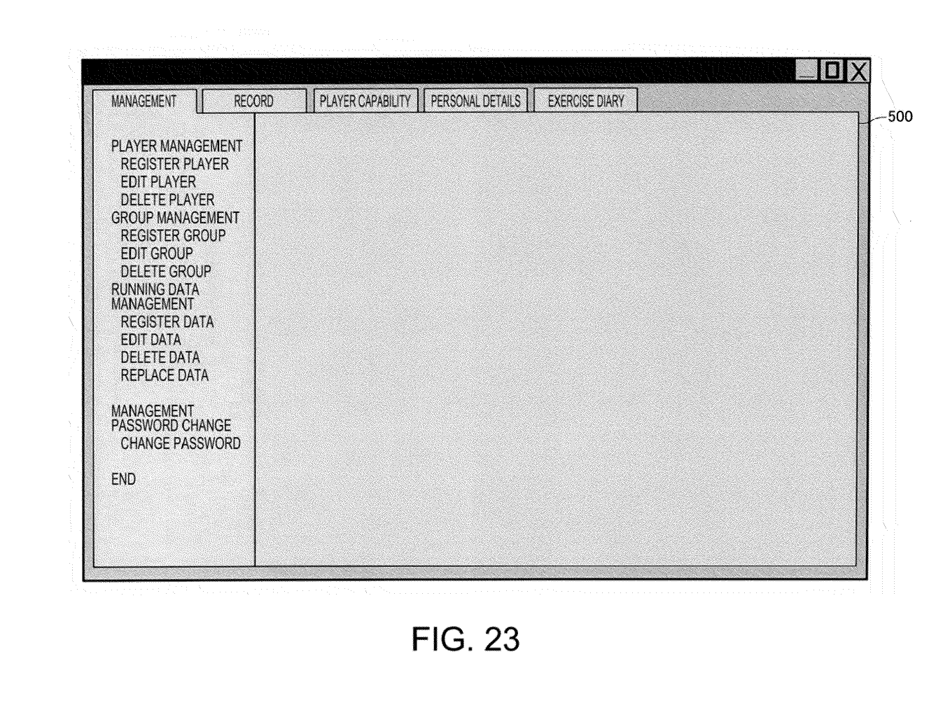

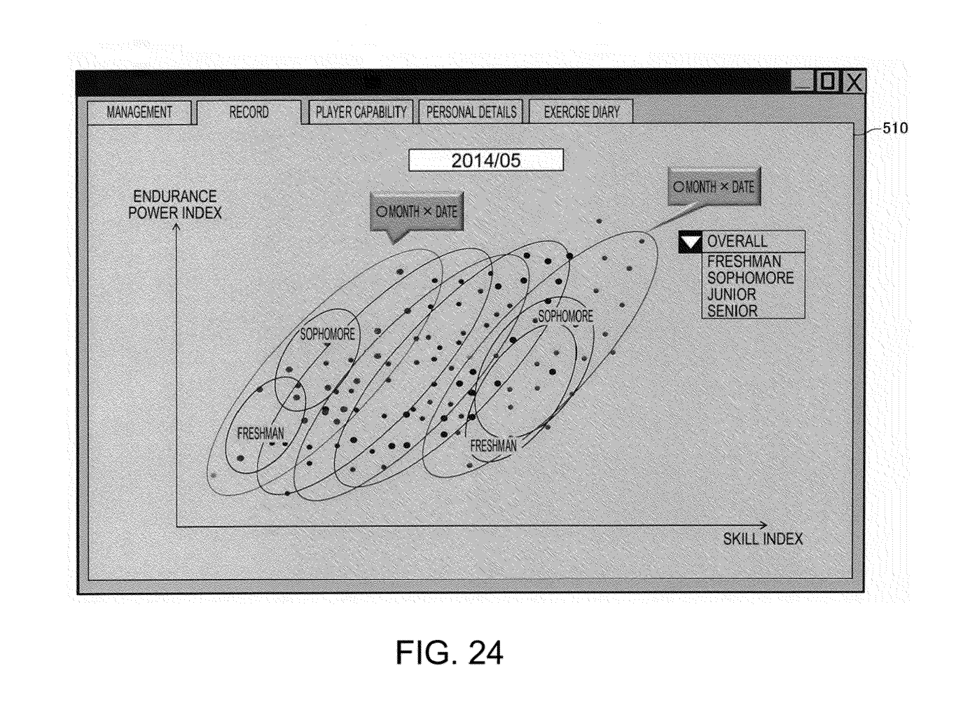

FIG. 24 is a diagram illustrating an example of a record tab screen. The record tab screen corresponds to a display screen for the analysis information in the overall analysis mode described above. As illustrated in FIG. 24, the record tab screen 510 includes a scatter diagram in which a horizontal axis indicates a skill index, a vertical axis indicates an endurance power index, and a skill index value and an endurance power index value in daily running of all players belonging to a selected group of a selected month are plotted. When the manager selects the month and the group in the record tab screen 510, the processing unit 420 acquires the exercise analysis information (the value of each exercise index) and the endurance power index value in all running that all players belonging to the selected group perform in the selected month from the database of the server 5. Also, the processing unit 420 daily calculates a skill index value for each player using the value of a predetermined exercise index, and generates a scatter diagram in which a horizontal axis indicates a skill index, and a vertical axis indicates an endurance power index.

The skill index is an index indicating skill power of the player and is calculated using, for example, skill index=stride/ground time/amount of work of one step. When a weight of the player is m and the 3-axis acceleration in the m frame is a, force F is F=ma, and the amount of work is calculated using Equation (7) for integrating an inner product F.v of the force F and the 3-axis speed v in the m frame. By integrating the inner product corresponding to one step, the amount of work of one step is calculated.

Amount of work=∫F·v dt (7)

Further, the endurance power index is, for example, a heart rate reserved (HRR), and is calculated as (heart rate−heart rate at rest)/(maximum heart rate−heart rate at rest)×100. A value of this endurance power index is registered as part of the running data in the database of the server 5 using any method. For example, the endurance power index value may be one of the exercise index value included in the exercise analysis information of the exercise analysis device 2 and may be registered in the database through the running data registration described above. In a specific method, for example, the reporting device 3 is manipulated to input the heart rate, the maximum heart rate, and the heart rate at rest each time each player runs, or the player wearing a heart rate meter runs, and the exercise analysis device 2 acquires values of the heart rate, the maximum heart rate, and the heart rate at rest from reporting device 3 or the heart rate meter to calculate the endurance power index value. The endurance power index value is set as one of the exercise index values included in the exercise analysis information.

In the example of FIG. 24, plots of skill index values and endurance power index values of all players of the team in each date which have run on May 2014 are surrounded by one ellipse, and plots of skill index values and endurance power index values of players belonging to the same group are daily surrounded by one ellipse. Further, the values may be plotted in different colors for each player or each group. A unit of display may be, for example, daily, monthly, and a yearly, and a plurality of units may be displayed.

The manager can confirm whether team power increases as a whole by viewing a change in the capability of all players of the team in the record tab screen 510. Further, a change in growth of the player is displayed as a list so that the capability of the entire team can recognized.

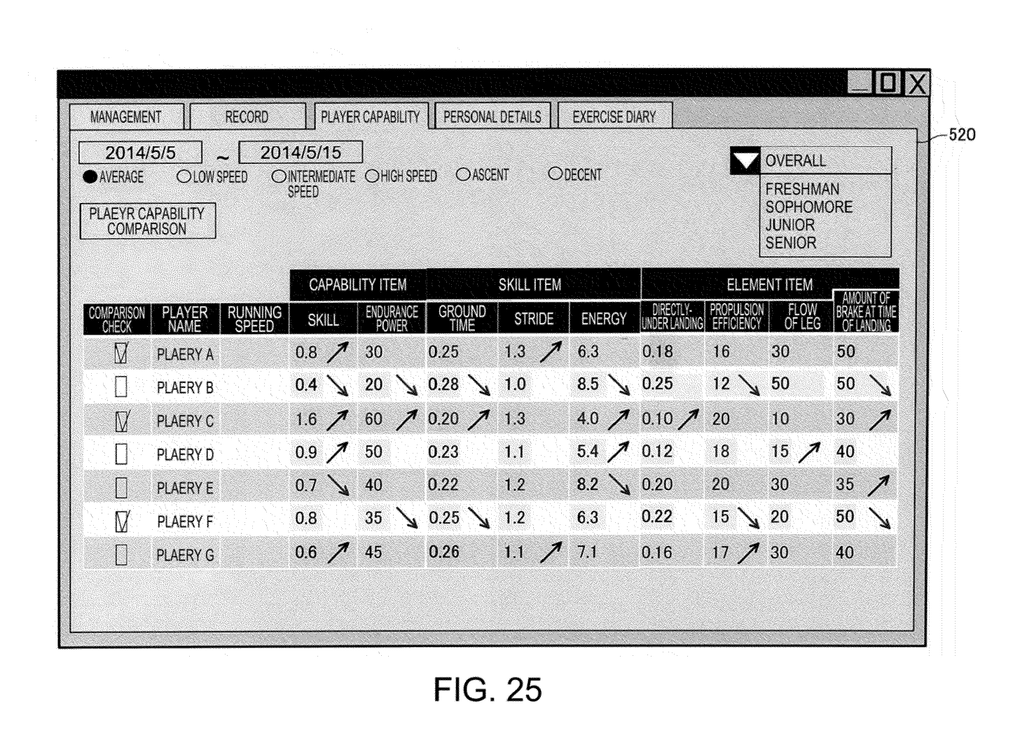

FIG. 25 is a diagram illustrating an example of a player capability tab screen. The player capability tab screen corresponds to the display screen for the analysis information in the overall analysis mode described above. As illustrated in FIG. 25, the player capability tab screen 520 includes a table in which an average value of a predetermined item in all running performed in a time period selected by all players belonging to the selected group is described. When the manager selects the time period and the group in the player capability tab screen 520, the processing unit 420 acquires the exercise analysis information (the value of each exercise index) and the endurance power index value in all running performed in the time period selected by all the players belonging to the selected group from the database of the server 5. Also, the processing unit 420 calculates the average value of each exercise index or the average value of the endurance power index for each player, calculates the average value of the skill index value of each player using the average value of a predetermined exercise index, and creates a table.

In the example illustrated in FIG. 25, the names of the players in the entire team, and respective average values of running speed, capability items (for example, skill index and endurance power index), skill items (for example, ground time, stride, and energy), and element items (for example, directly-under landing rate (directly-under landing rate 3), propulsion efficiency, a flow of a leg, and an amount of brake at the time of landing) in all running performed in May 5 to May 15, 2014 are displayed. Further, particularly good values or bad values may be displayed in different colors or may be displayed with gray when the running time is short or reliability is low. Further, a recent trend toward improvement may be displayed by an arrow or an icon. Further, various sorting functions of performing displaying in a good order when each item is clicked may be included. Further, an average value of each item at “low speed (for example, 0 to 2 m/sec),” “intermediate speed (for example, 2 to 5 msec)”, and “high speed (for example, 5 to 10 msec) may be displayed in consideration of a change in a running way of each player according to the speed. An average value of each item in “ascent (for example, an altitude difference is +0.5 msec or more)”, and “descent (for example, the altitude difference is −0.5 msec or more)” may be displayed in consideration of a change in the running way of each player according to a situation of a running road.

The manager can understand at a glance whether each player has strength or weakness in any of the skill and the endurance in the player capability tab screen 520, and can perform detailed analysis as to whether each player has strength or weakness for which skill item or whether the player has strength or weakness for which element item constituting the skill item. Thus, the manager can introduce training suitable for each player. For example, since the respective elements (the just directly-under landing, the propulsion efficiency, the flow of the leg, and the amount of brake at the time of landing) for shortening the ground time is converted into a numerical value, the items for exercise become clear. Further, the manager can recognize a trend toward improvement of the players and confirm validity of the exercise.

In the example illustrated in FIG. 25, a comparison check box is provided at the left end of the table. When the manager checks in the comparison check box and presses the player capability comparison button, a player capability comparison screen is displayed, and the running capabilities can be compared between selected players.

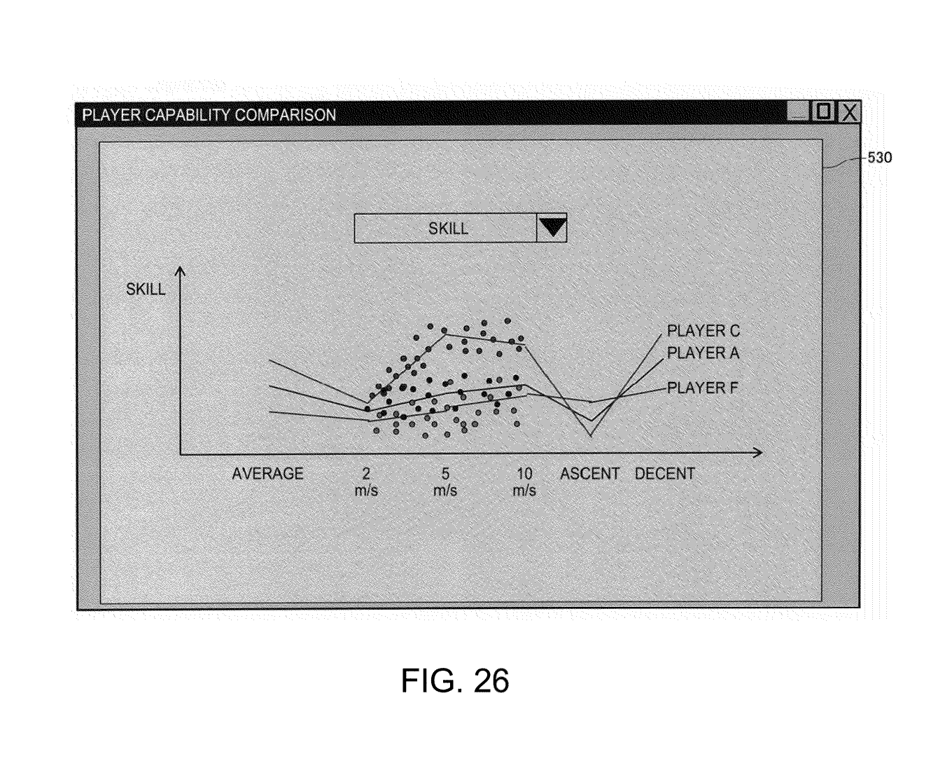

FIG. 26 is a diagram illustrating an example of a player capability comparison screen. The player capability comparison screen corresponds to a display screen for the analysis information in the overall analysis mode described above. As illustrated in FIG. 26, the player capability comparison screen 530 includes a graph in which a value of a selected item of a selected player is plotted for “average”, “low speed (0˜2 m/s)”, “intermediate speed (2˜5 m/s)”, “high speed (5˜10 m/s)”, “ascent”, and “descent”. When the manager selects the item in the player capability comparison screen 530, the processing unit 420 calculates an average value of all running in the selected period, an average of the ascent of all running, an average value of the descent of the total running, and an average value of each constant speed between low speed and high speed of each running for the selected item for each selected player, and plots the average values to create a scatter diagram.

In the example illustrated in FIG. 26, the average value in all running performed on May 5 to May 15, 2014, the average value of the ascent in all running, the average value of the descent in all running, and the average value at each constant speed between the speed 2 m/s and 10 m/s in each running are sequentially plotted for a skill index of players A, C, and F. Further, for the respective players A, C, and F, an approximation curve generated by a least squares method or the like, and a line graph in which respective plots of the average value in all running, the average value of the ascent in all running value, and the average value of the descent in all running are connected is displayed for the skill index value between 2 m/s and 10 m/s. Further, the respective players may be displayed in different colors. Further, a plurality of such graphs may be simultaneously displayed with a changed item so that a correlation between the plurality of items is easily understood.

The manager can clarify strength and weakness of each player by comparing all average values, average values at each speed, average values in the ascent, and average values in the descent for the selected item between the selected players in the player capability comparison screen 530 at the same time. Further, since the average values at the respective speeds are sequentially displayed, the manager can also discover the speed at which each player is weak, for the selected item.

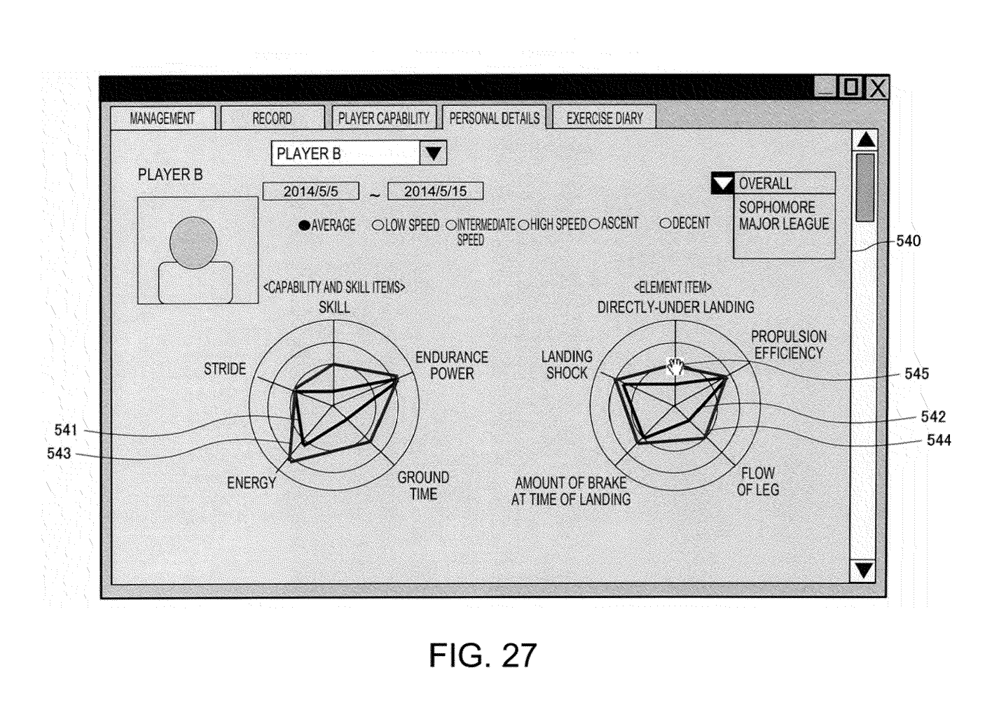

FIGS. 27 to 32 are diagrams illustrating an example of a personal detail tab screen. The personal detail tab screen corresponds to the display screen for the analysis information in the above-described personal analysis mode. FIG. 27 is a diagram illustrating an example of a capability level screen that is a screen of a first page of the personal detail tab screen. As illustrated in FIG. 27, the capability level screen 540 includes a radar chart showing relative evaluation, in a selected group, of a capability item and a skill item in running in the time period selected by the selected player, and a radar chart showing relative evaluation, in the selected group, of an element item in running in a time period selected by the selected player. When the manager or the player selects the player, the period, and the group in the capability level screen 540, the processing unit 420 acquires the exercise analysis information (the value of each exercise index) and the endurance power index value in all running performed in the selected period by all players belonging to the selected group from the database of the server 5. Also, the processing unit 420 calculates the average value of each exercise index or the average value of the endurance power index of each player, sets a maximum value in the selected group to 10 and a minimum value to 0 for the value of each item (each index value), converts the value of the selected player into a relative evaluation value, and generates two radar charts.

In the example of FIG. 27, two radar charts in which each index value of the player B is relatively evaluated based on an image of the selected player B, capability items (for example, skill index, and endurance power index), skill items (for example, ground time, stride, and energy), element items (for example, directly-under landing, propulsion efficiency, flow of the leg, amount of brake at the time of landing, and landing shock) in all running performed by the players of entire team on May 5 to May 15, 2014 are displayed. For each index value shown by the radar chart, any one of “average”, “low speed”, “intermediate speed”, “high speed”, “ascent”, and “decent” can be selected. Further, since player B belongs to the group “sophomore” and belongs to the group “major league”, any one of “all”, “sophomore”, and “major league” can be selected as the group.

In the capability level screen 540, the target value of each index can be set. In the example of FIG. 27, in the two radar charts, line segments (for example, black line segments) 541 and 542 close to centers connect five points indicating the values of the five indexes, and other line segments (for example, red line segments) 543 and 544 connect five points indicating target values of the five indexes. In the radar chart of the capability items and the skill items on the left side, the target values of the skill index, the ground time, and the energy are set higher than the current values. In the radar chart in the element items on the right side, the target values of the four indexes other than the propulsion efficiency are set higher than the current values. The setup of the target value of each index can be changed by grabbing the point indicating each index value using a cursor 545 of a mark of a hand and copying and moving the value (dragging).

When the manager or the player sets the target value of each index in the capability level screen 540, the processing unit 420 acquires the information of the set target value of each index and stores the information in the storage unit 430. As described above, this target value is sent to the reporting device 3 and compared with each index value included in the output information during running in the reporting device 3.

Each player can recognize whether a position of the player or a certain item in the team (in the group) is to be primarily improved, in the capability level screen 540. Further, each player can set the target together with a supervisor or a coach while viewing a difference with the other player in the capability level screen.

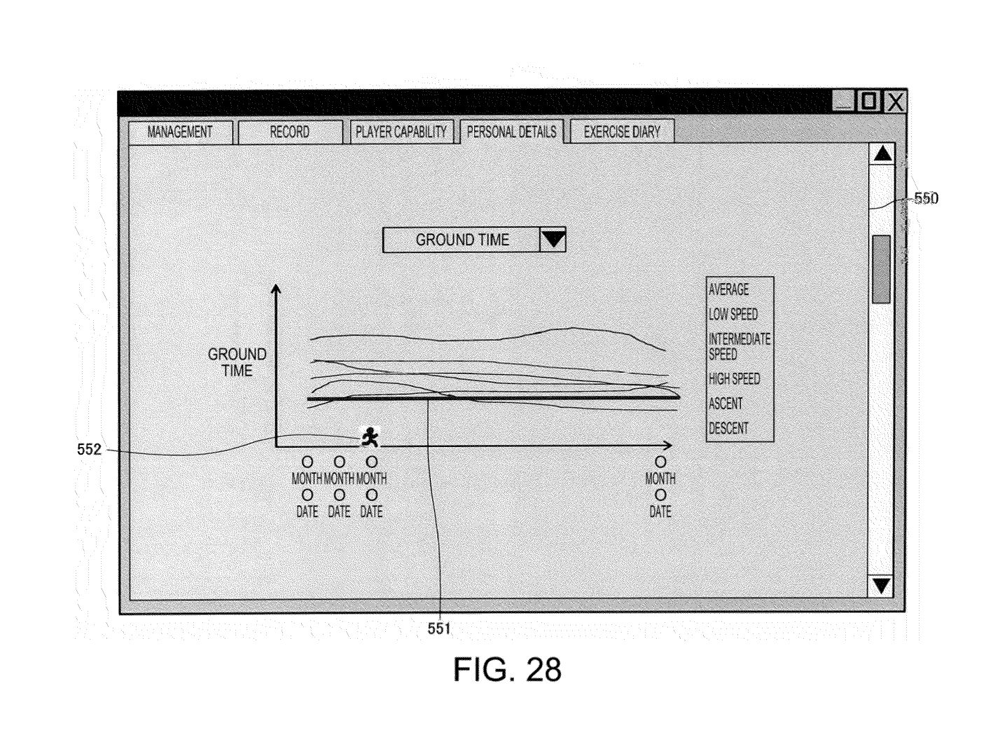

FIG. 28 is a diagram illustrating an example of a capability transition screen that is a screen of a second page of the personal detail tab screen. As illustrated in FIG. 28, the capability transition screen 550 includes a time-series graph of the selected index in the running of the period (May 5 to May 15, 2014) of a selected player (player B) selected in the capability level screen 540 (the screen of the first page of the personal detail tab screen). A horizontal axis of this time-series graph indicates a time (date), and a vertical axis indicates a value of the selected index. When the manager or the player selects the index in the capability transition screen 550, the processing unit 420 converts the value of the index selected by the selected player into a relatively evaluation value per date to create a time-series graph, as described above.

In the example of FIG. 28, five line graphs showing relative evaluation values within a team of a ground time in each of “average”, “low speed”, “intermediate speed”, “high speed”, “ascent”, and “descent” of the selected player B in time series are displayed side by side. However, the graph to be displayed may be selectable. Further, a time-series graph 551 of the target value (for example, a red bold line) may be displayed. Further, for example, on the date of an official meet (race), a mark 552 indicating that the running of the day is the official meet (race) (for example, a mark imitating a state in which the human is running) may be attached. Further, when the cursor contacts the date, a memo of the exercise diary (which will be described below) may also be displayed. Further, a plurality of graphs of each index value may be displayed at the same time.

Each player can recognize a trend of a degree of improvement due to exercise in the capability transition screen 550. Further, each player can determine whether the exercise is effective or consciousness of the player is correct by simultaneously viewing the exercise memo or the time-series graph.

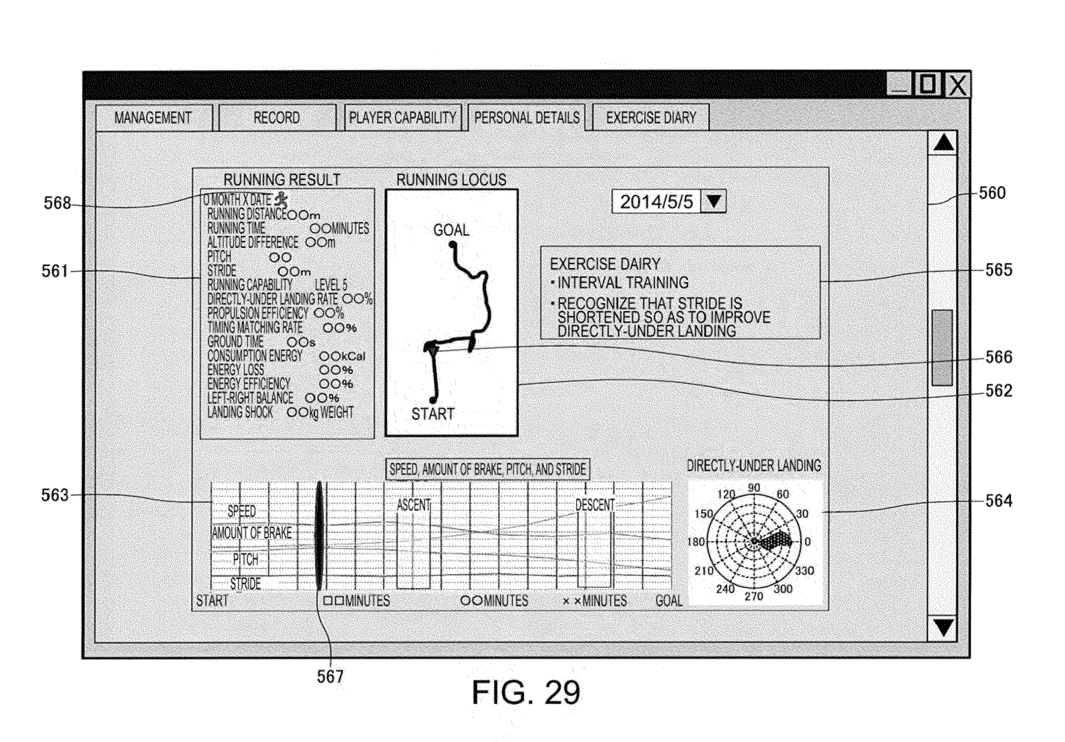

FIG. 29 is a diagram illustrating an example of a running transition screen that is a screen of a third page of the personal detail tab screen. As illustrated in FIG. 29, the running transition screen 560 includes, for example, running result information 561 in running on date selected by a player (player B) selected in a capability level screen (screen of a first page of the personal detail tab screen), an image 562 showing a running locus, a first graph 563 showing values of some elements included in the running result in time series from start to a goal, a second graph 564 showing the values of some elements included in the running result to be easily understood, and information 565 on a memo of an exercise diary. When the manager or the player selects date in the running transition screen 560, the processing unit 420 creates the running result information 561, the running locus image 562, the first graph 563, and the second graph 564 using the running data in the selected date of the selected player, and acquires the information 565 on the memo of exercise diary registered in association with the running data from the database of the server 5.

In the example illustrated in FIG. 29, the running result information 561 on May 5, 2014 of the selected player B, the image 562 showing the running locus on May 5, 2014, the first graph 563 showing values of respective elements of “speed”, “amount of brake”, “pitch”, and “slide” in time series, the second graph 564 showing directly-under landing, and the information 565 on the memo of the exercise diary on May 5, 2014 are displayed. The second graph 564 is a graph showing the directly-under landing to be easily recognized by plotting all landing positions during running, with a center of a circle being directly under the body of the player B, and a right direction being the running direction.

When running in the selected date is an official meet (race), a mark 568 (a mark imitating a state in which a person runs) indicating that the running is the official meet (race) is added next to the date of the running result. Further, in an image 562 showing a running locus, a mark 566 (for example, mark V) indicating a current position that is movable through dragging using the cursor may be displayed, and the value of each element of information 561 on the running result may be changed in conjunction with the mark 566. Further, in the first graph 563, a slide bar 567 indicating a current time that is movable through dragging using the cursor may be displayed, and the value of each element of the information 561 on the running result may be changed in conjunction with a position of the slide bar 567. When one of the mark 566 in the image 562 showing the running locus and the slide bar 567 in the first graph 563 is moved, a position of the other may be accordingly changed. Further, the element name of the information 561 on the running result may be dragged using the cursor and dropped in the display area of the first graph 563 or the second graph 564, or the element in the first graph 563 or the second graph 564 may be deleted so that a display target of the first graph 563 or the second graph 564 is selectable. Further, in the first graph 563, a period of “ascent” or “descent” may be recognized. Further, the running transition screens 560 of a plurality of players can be displayed at the same time.

Each player can perform analysis of the running of the player using the running transition screen 560. For example, each player can recognize causes of low speed in the second half from the element.

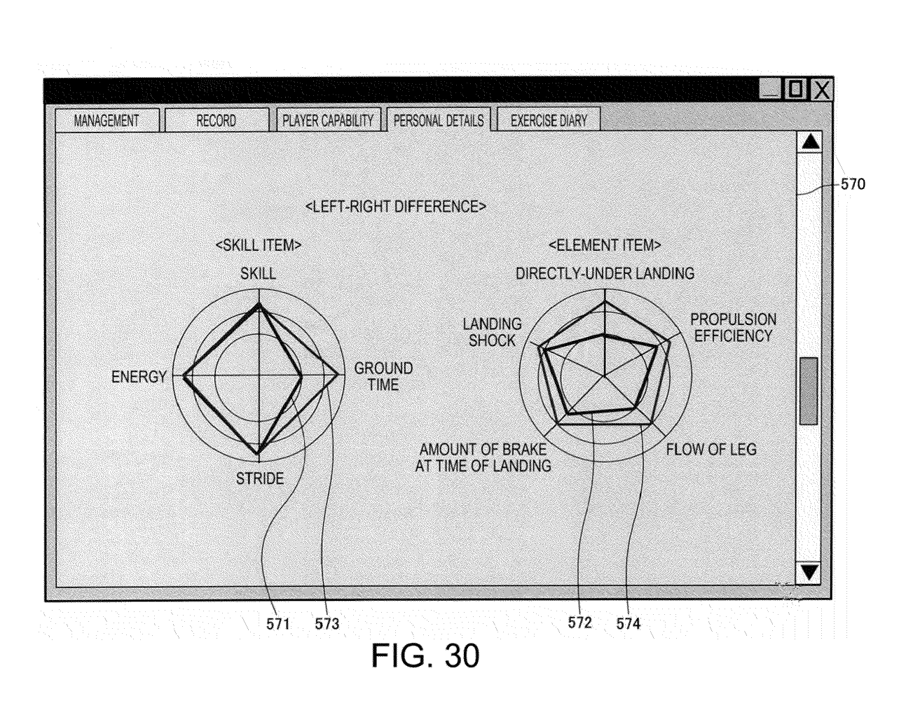

FIG. 30 is a diagram illustrating an example of a left-right difference screen that is a screen of a fourth page of the personal detail tab screen. As illustrated in FIG. 30, the left-right difference screen 570 includes a radar chart in which a skill index and each index value of the skill item in running of a selected time period (May 5˜May 15, 2014) of a selected player (player B) in the capability level screen 540 (a screen of a first page of the personal detail tab screen) are relatively evaluated at left and right within a selected group, and a radar chart in which each index value of the element item in running of the selected time period of the selected player is relatively evaluated at left and right within the selected group.

In the example of FIG. 30, two radar charts indicating right and left values of each index of player B based on left and right values of the skill index, and right and left values of each index of skill items (for example, ground time, stride, and energy), and element items (for example, directly-under landing, propulsion efficiency, a flow of a leg, an amount of brake at the time of landing, and landing shock) are displayed. In the two radar charts, line segments 571 and 572 (for example, green lines) that connect plots showing values of the left foot of the respective indexes, and line segments 573 and 574 (for example, red lines) that connect plots showing values of the right foot of the respective indexes are divided according to colors. When a cursor is put in a display position of each index name, the value of the left foot and the value of the right foot may be displayed simultaneously. Further, in these radar charts, target values of the right and left values of each index can be set, similar to the radar charts of the capability level screen 540.

Each player can recognize what percentage a difference between left and right of each index is in the left-right difference screen 570 and utilize this for exercise or training. Further, each player can aim at elimination of the difference between right and left from the viewpoint of injury prevention.

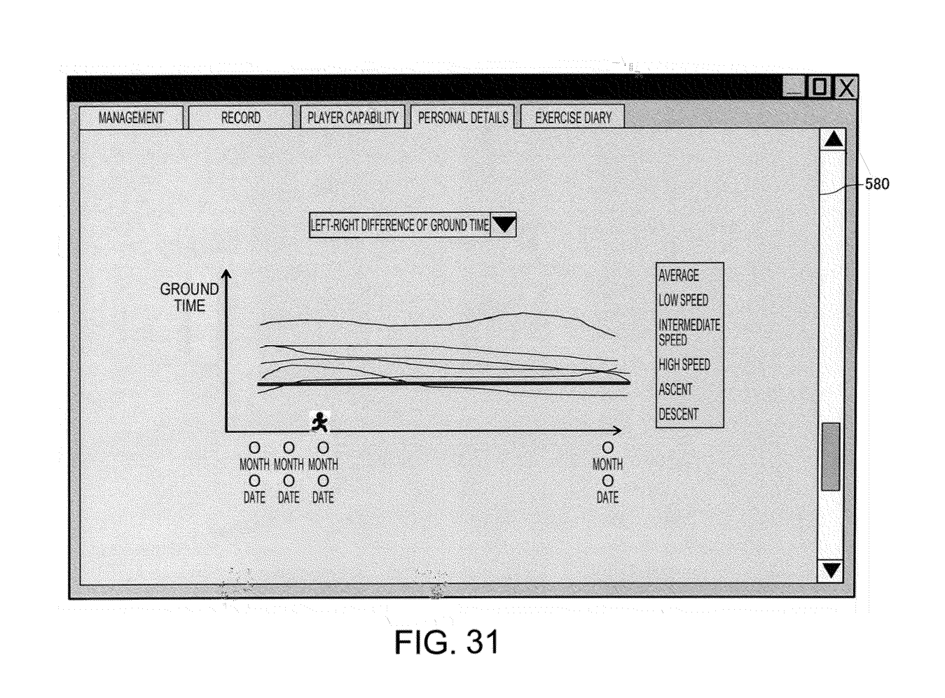

FIG. 31 is a diagram illustrating an example of a left-right difference transition screen that is a fifth page of the personal detail tap screen. As illustrated in FIG. 31, the left-right difference transition screen 580 includes a time-series graph showing a difference between right and left of a selected index in running of the selected time period (May 5 to May 15, 2014) of the selected player (player B) in the capability level screen 540 (the screen of the first page of the personal detail tab screen). Since this left-right difference transition screen 580 is the same as the capability transition screen 550 (see FIG. 28) except that the time-series graph of the left-right difference of the selected index is displayed, description thereof will be omitted.

Each player can recognize a trend toward a degree of improvement of the left-right difference due to exercise in the left-right difference transition screen 580. Further, each player can determine whether the exercise is effective or consciousness of the player is correct by simultaneously viewing the exercise memo or the time-series graph. Further, each player can confirm whether there is no abrupt change in the left-right difference to prevent injury.

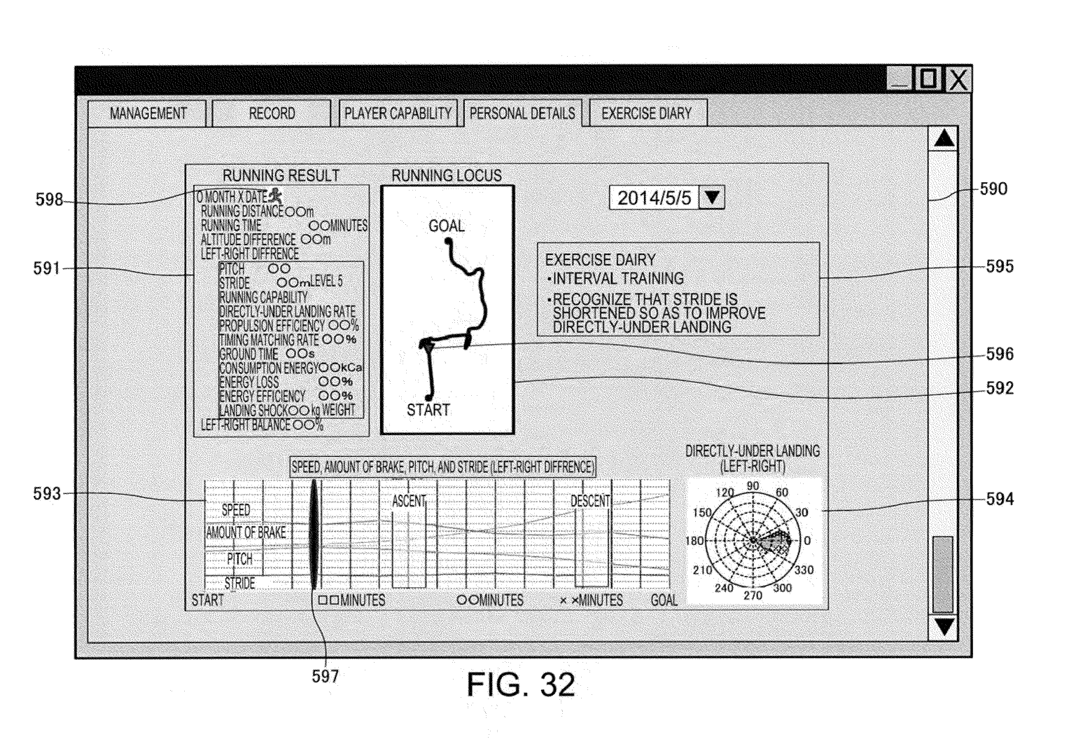

FIG. 32 is a diagram illustrating an example of a left-right running difference transition screen that is a sixth page of the personal detail tap screen. As illustrated in FIG. 32, the left-right running difference transition screen 590 includes, for example, information 591 on a running result in which the value of the left-right difference of each index is included in running of a selected date of a selected player (player B) in the capability level screen 540 (the screen of the first page of the personal detail tab screen), an image 592 indicating a running locus, a first graph 593 showing values of the left-right differences of some elements included in the running result in time series from start to a goal, a second graph 594 showing the right and left values of some elements included in the running result to be easily understood, and information 595 on a memo of the exercise diary.

In the example of FIG. 32, information 591 on a running result on May 5, 2014 (a value of a difference between right and left of each index is included), an image 592 showing a running locus on May 5, 2014, a first graph 593 showing the value of the left-right difference of each element of “speed”, “amount of brake”, “pitch”, and “slide” in time series, a second graph 594 showing directly-under landing in different colors at the left and right, and information 595 on a memo of an exercise diary on May 5, 2014 of the selected player B are displayed. Since another configuration of the left-right running difference transition screen 590 is the same as the running transition screen 560 (see FIG. 29), description thereof will be omitted.

Each player can perform analysis of running of the player in the left-right running difference transition screen 590. For example, since the difference between right and left increases in the second half, each player can carefully exercise. Further, each player can confirm whether there is no abrupt change in the left-right difference to prevent injury.

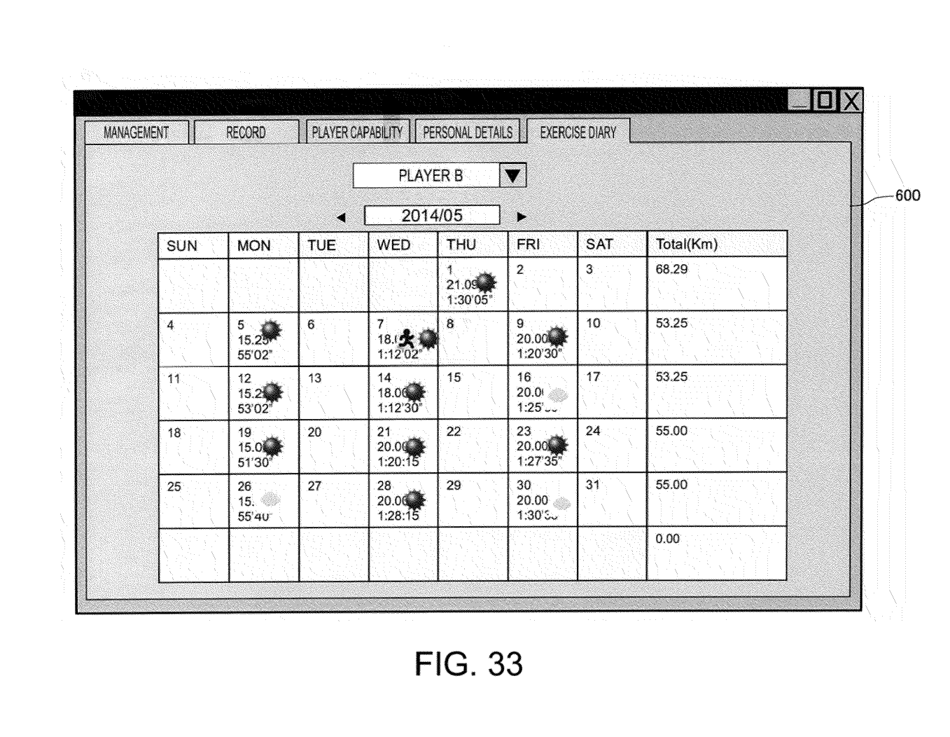

FIG. 33 is a diagram illustrating an example of the exercise diary tab screen. As illustrated in FIG. 33, an exercise diary tab screen 600 includes a calendar in which, for example, an overview (running distance or time on each date) of the running result in the selected month of the selected player is described. When the manager or the player clicks on the calendar date, the memo of the exercise diary of the day is displayed when there is the memo. The manager or the player can create and edit the memo of the exercise diary. Further, a mark 601 (for example, a mark imitating a state in which a person runs) indicating that the running is an official meet (race) is displayed in the calendar. Further, when the manager or the player clicks on the date of the exercise diary, the screen may be shifted to the running transition screen 560 in which the date is selected (see FIG. 29).

When the manager or the player selects the player and the month in the exercise diary tab screen 600, the processing unit 420 acquires information such as running date, distance, time, weather, and official meet (race) of all running data in the selected month of the selected player from the database of the server 5, and acquires memo information of the exercise diary registered in association with the running data from the database of the server 5. Also, the processing unit 420 creates a calendar using each piece of information of the acquired running data, and links the memo information of the exercise diary to date of the calendar.

The manager or the player can recognize exercise content in the exercise diary tab screen 600. Further, the manager or the player can write a memo about exercise content or his or her recognition during the exercise in the exercise diary tab screen 600, and can confirm whether there are effects from a change in the capability items, the skill items, and the element items in other screens.

### 1-6. Effects

According to the first embodiment, since the inertial measurement unit 10 can detect a fine motion of the torso of the user using the 3-axis acceleration sensor 12 and the 3-axis angular speed sensor 14, the exercise analysis device 2 can accurately analyze the running exercise using the detection result of the inertial measurement unit 10 during running of the user. Therefore, according to the first embodiment, the information analysis device 4 can generate the analysis information from which the running capabilities of the plurality of users can be compared using the exercise analysis information of the plurality of users generated by one or a plurality of exercise analysis devices 2, and present the analysis information. Each user can compare the running capability of the user with the running capability of other users using the presented analysis information.

Further, according to the first embodiment, since the information analysis device 4 generates analysis information from which running capabilities of the plurality of users are comparable on each date on which the plurality of users who are analysis targets perform running in the overall analysis mode, each user can recognize a transition of the difference with the running capacity of the other users using the presented analysis information.

Further, according to the first embodiment, since the information analysis device 4 generates analysis information from which running capabilities of the plurality of users who are analysis targets are comparable for each group in the overall analysis mode, each user can compare running capability of the user with running capabilities of other users belonging to the same group as the user using the presented analysis information.

Also, according to the first embodiment, since the information analysis device 4 can generate the analysis information from which the value of the exercise index of any user included in the plurality of users can be relatively evaluated, using the values of the exercise indexes of the plurality of users who are analysis targets in the personal analysis mode, the user can relatively evaluate the running capability of the user among the plurality of users using the presented analysis information. Further, the user can appropriately set the target values of each index according to the exercise capability of the user while viewing the value of the relatively evaluated exercise index.

Further, according to the first embodiment, since the information analysis device 4 generates the analysis information from which the values of various exercise indexes of any user is comparable with the respective target values in the personal analysis mode, the user can recognize the difference between the running capability of the user and the target using the presented analysis information.

Further, according to the first embodiment, since the reporting device 3 compares the value of each exercise index during running of the user with the target value set based on the analysis information of the past running, and reports the comparison result to the user through sound or vibration, the user can recognize the goodness or badness of each exercise index in real time without the running being obstructed. Thus, for example, the user can run through trial and error to achieve the target value or can run while recognizing the exercise indexes in question when the user is tired.

### 1. Second Embodiment

In a second embodiment, the same components as those in the first embodiment are denoted with the same reference numerals, and description thereof will be omitted or simplified. Different content from that in the first embodiment will be described in detail.

### 2-1. Configuration of Exercise Analysis System

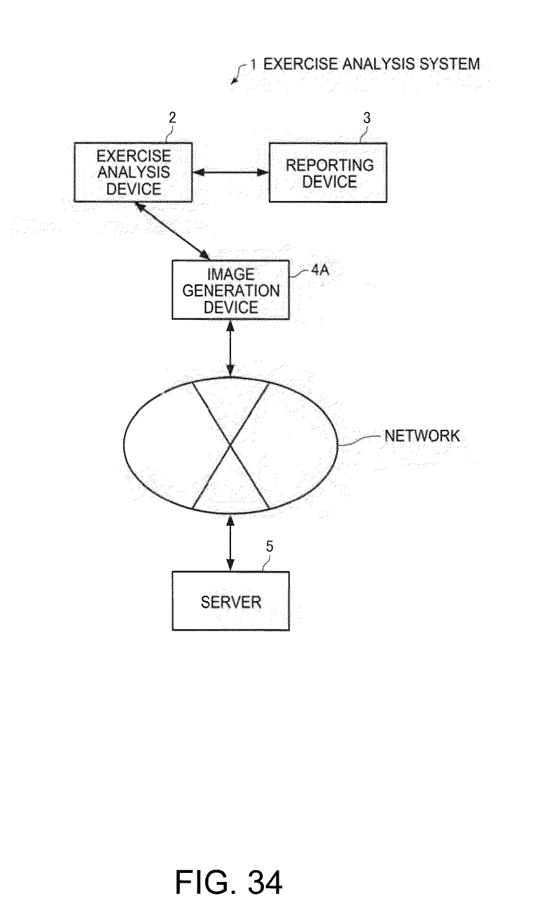

Hereinafter, an exercise analysis system that analyzes exercise in running (including walking) of a user will be described by way of example, but an exercise analysis system of a second embodiment may be an exercise analysis system that analyzes exercise other than running. FIG. 34 is a diagram illustrating an example of a configuration of an exercise analysis system 1 of the second embodiment. As illustrated in FIG. 34, the exercise analysis system 1 of the second embodiment includes an exercise analysis device 2, a reporting device 3, and an image generation device 4A. The exercise analysis device 2 is a device that analyzes exercise during running of the user, and the reporting device 3 is a device that notifies the user of information on a state during running of the user or a running result, similar to the first embodiment. The image generation device 4A is a device that generates image information on a running state (an example of an exercise state) of the user using information of an analysis result of the exercise analysis device 2, and is referred to as an information analysis device that analyzes and presents the running result after the running of the user ends. In the second embodiment, as illustrated in FIG. 2, the exercise analysis device 2 includes an inertial measurement unit (IMU) 10, and is mounted to a torso portion (for example, a right waist, a left waist, or a central portion of a waist) of the user so that one detection axis (hereinafter referred to as a z axis) of the inertial measurement unit (IMU) 10 substantially matches a gravitational acceleration direction (vertically downward) in a state in which the user is at rest, similar to the first embodiment. Further, the reporting device 3 is a wrist type (wristwatch type) portable information device, and is mounted on, for example, the wrist of the user, similar to the first embodiment. However, the reporting device 3 may be a portable information device, such as a head mount display (HMD) or a smartphone.

The user operates the reporting device 3 at the time of running start to instruct the exercise analysis device 2 to start measurement (inertial navigation operation process and exercise analysis process to be described below), and operates the reporting device 3 at the time of running end to instruct to end the measurement in the exercise analysis device 2, similar to the first embodiment. The reporting device 3 transmits a command for instructing start or end of the measurement to the exercise analysis device 2 in response to the operation of the user.

Similar to the first embodiment, when the exercise analysis device 2 receives the measurement start command, the exercise analysis device 2 starts the measurement using an inertial measurement unit (IMU) 10, calculates values for various exercise indexes which are indexes regarding running capability (an example of exercise capability) of the user using a measurement result, and generates exercise analysis information including the values of the various exercise indexes as information on the analysis result of the running exercise of the user. The exercise analysis device 2 generates information to be output during running of the user (output information during running) using the generated exercise analysis information, and transmits the information to the reporting device 3. The reporting device 3 receives the output information during running from the exercise analysis device 2, compares the values of various exercise indexes included in the output information during running with respective previously set target values, and reports goodness or badness of the exercise indexes to the user through sound or vibration. Thus, the user can run while recognizing the goodness or badness of each exercise index.

Further, similar to the first embodiment, when the exercise analysis device 2 receives the measurement end command, the exercise analysis device 2 ends the measurement of the inertial measurement unit (IMU) 10, generates user running result information (running result information: running distance and running speed), and transmits the user running result information to the reporting device 3. The reporting device 3 receives the running result information from the exercise analysis device 2, and notifies the user the running result information as a text or an image. Accordingly, the user can recognize the running result information immediately after the running end. Alternatively, the reporting device 3 may generate the running result information based on the output information during running, may notify the user of the running result information as a text or an image.

Also, data communication between the exercise analysis device 2 and the reporting device 3 may be wireless communication or may be wired communication.

Further, in the second embodiment, the exercise analysis system 1 includes a server 5 connected to a network, such as the Internet or a LAN, as illustrated in FIG. 34, similar to the first embodiment. An image generation device 4A is, for example, an information device such as a personal computer or a smart phone, and can perform data communication with the server 5 over the network. The image generation device 4A acquires the exercise analysis information in past running of the user from the exercise analysis device 2, and transmits the exercise analysis information to the server 5 over the network. However, a device different from the image generation device 4A may acquire the exercise analysis information from the exercise analysis device 2 and transmit the exercise analysis information to the server 5 or the exercise analysis device 2 may directly transmit the exercise analysis information to the server 5. The server 5 receives this exercise analysis information and stores the exercise analysis information in a database built in a storage unit (not illustrated).

The image generation device 4A acquires the exercise analysis information of the user at the time of running, which is generated using the measurement result of the inertial measurement unit (IMU) 10 (an example of the detection result of the inertial sensor), and generates image information in which the acquired exercise analysis information is associated with the image data of the user object indicating the running of the user. Specifically, the image generation device 4A acquires the exercise analysis information of the user from the database of the server 5 over the network, generates image information on the running state of the user using the values of various exercise indexes included in the acquired exercise analysis information, and displays the image information on the display unit (not illustrated in FIG. 34). The running capability of the user can be evaluated from the image information displayed on the display unit of the image generation device 4A.

In the exercise analysis system 1, the exercise analysis device 2, the reporting device 3, and the image generation device 4A may be separately provided, the exercise analysis device 2 and the reporting device 3 may be integrally provided and the image generation device 4A may be separately provided, the reporting device 3 and the image generation device 4A may be integrally provided and the exercise analysis device 2 may be separately provided, the exercise analysis device 2 and the image generation device 4A may be integrally provided and the reporting device 3 may be separately provided, and the exercise analysis device 2, the reporting device 3, and the image generation device 4A may be integrally provided. The exercise analysis device 2, the reporting device 3, and the image generation device 4A may be any combination.

### 2-2. Coordinate System

A coordinate system required in the following description is defined as in “1-2. Coordinate system” of the first embodiment.

### 2-3. Exercise Analysis Device

### 2-3-1. Configuration of the Exercise Analysis Device

Since an example of a configuration of the exercise analysis device 2 of the second embodiment is the same as that in the first embodiment (FIG. 3), description thereof will not be illustrated. In the exercise analysis device 2 of the second embodiment, since respective functions of the inertial measurement unit (IMU) 10, the storage unit 30, the GPS unit 50, and the geomagnetic sensor 60 are the same as those in the first embodiment, description thereof will be omitted.

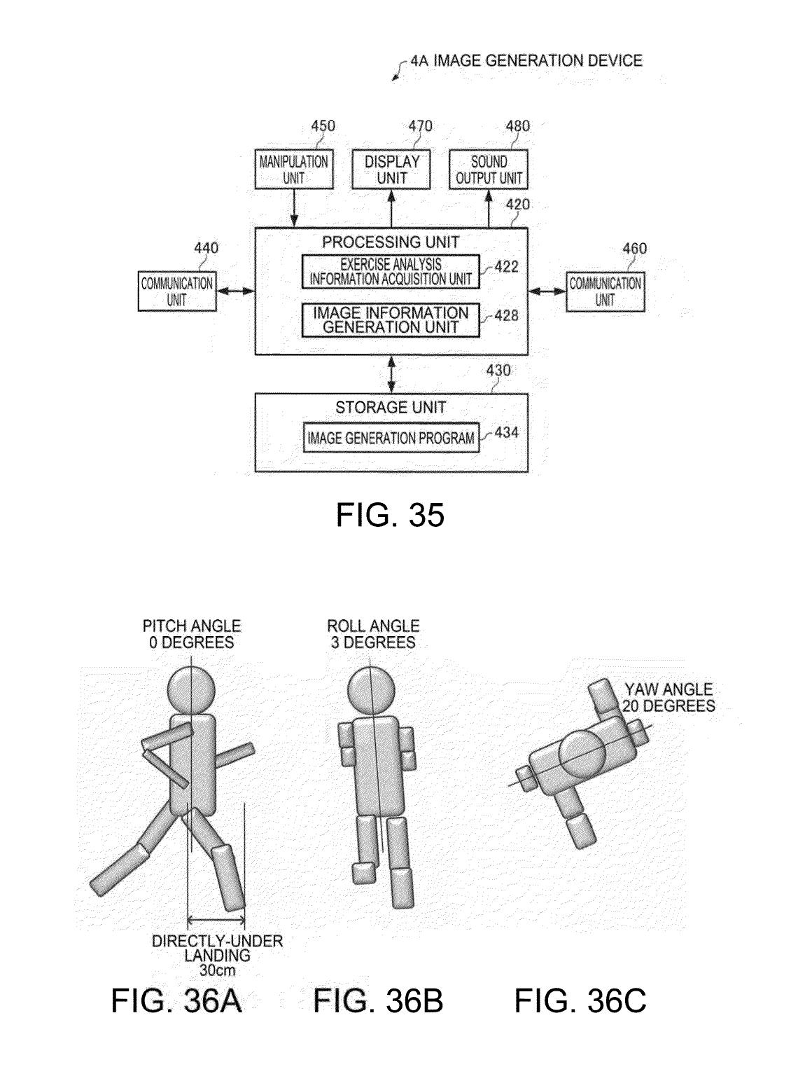

The communication unit 40 is a communication unit that performs data communication with the communication unit 140 of the reporting device 3 (see FIG. 18) or the communication unit 440 of the image generation device 4A (see FIG. 35), and performs, for example, a process of receiving a command (for example, measurement start/measurement end command) transmitted from the communication unit 140 of the reporting device 3 and sending the command to the processing unit 20, a process of receiving the output information during running or the running result information generated by the processing unit 20 and transmitting the information to the communication unit 140 of the reporting device 3, or a process of receiving a transmission request command for exercise analysis information from the communication unit 440 of the image generation device 4A, sending the transmission request command to the processing unit 20, receiving the exercise analysis information from the processing unit 20, and transmitting the exercise analysis information to the communication unit 440 of the image generation device 4A.

The processing unit 20 includes, for example, a CPU, a DSP, or an ASIC, and performs various operation processes or control processes according to various programs stored in the storage unit 30 (storage medium), similar to the first embodiment.

Further, when the processing unit 20 receives the transmission request command for the exercise analysis information from the image generation device 4A via the communication unit 40, the processing unit 20 performs a process of reading the exercise analysis information designated by the transmission request command from the storage unit 30, and sending the exercise analysis information to the communication unit 440 of the image generation device 4A via the communication unit 40.

### 2-3-2. Functional Configuration of the Processing Unit

Since an example of a configuration of the processing unit 20 of the exercise analysis device 2 in the second embodiment is the same as that in the first embodiment (FIG. 8), the example is not illustrated. In the second embodiment, the processing unit 20 executes the exercise analysis program 300 stored in the storage unit 30 to function as an inertial navigation operation unit 22 and an exercise analysis unit 24, similar to the first embodiment. Since each of functions of the inertial navigation operation unit 22 and the exercise analysis unit 24 is the same as that in the first embodiment, description thereof will be omitted.

### 2-3-3. Functional Configuration of the Inertial Navigation Operation Unit

Since an example of a configuration of the inertial navigation operation unit 22 in the second embodiment is the same as that in the first embodiment (FIG. 9), the example is not illustrated. In the second embodiment, the inertial navigation operation unit 22 includes a bias removal unit 210, an integration processing unit 220, an error estimation unit 230, a running processing unit 240, and a coordinate transformation unit 250, similar to the first embodiment. Respective functions of these components are the same as those in the first embodiment, description thereof will be omitted.

### 2-3-4. Functional Configuration of the Exercise Analysis Device

Since an example of a configuration of the exercise analysis unit 24 in the second embodiment is the same as that in the first embodiment (FIG. 13), the example is not illustrated. In the second embodiment, the exercise analysis unit 24 includes a feature point detection unit 260, a ground time and shock time calculation unit 262, a basic information generation unit 272, a first analysis information generation unit 274, a second analysis information generation unit 276, a left-right difference ratio calculation unit 278, and an output information generation unit 280, similar to the first embodiment. Since respective functions of these components are the same as those in the first embodiment, description thereof will be omitted.

### 2-3-5. Input Information

Since details of each item of input information has been described in “1-3-5. Input information” in the first embodiment, a description thereof will be omitted here.

### 2-3-6. First Analysis Information

Since details of each item of the first analysis information calculated by the first analysis information generation unit 274 has been described in “1-3-6. First analysis information” in the first embodiment, a description thereof will be omitted here.

### 2-3-7. Second Analysis Information

Since details of each item of the second analysis information calculated by the second analysis information generation unit 276 have been described in “1-3-7. Second analysis information” in the first embodiment, description thereof will be omitted here.

### 2-3-8. Left-Right Difference Ratio (Left-Right Balance)

Since details of the left-right difference ratio calculated by the left-right difference ratio calculation unit 278 have been described in “1-3-8. Left-right difference ratio (left-right balance)” in the first embodiment, description thereof will be omitted here.

### 2-3-9. Procedure of the Process

Since a flowchart illustrating an example of a procedure of the exercise analysis process performed by the processing unit 20 in the second embodiment is the same as that in the first embodiment (FIG. 14), the flowchart will not be illustrated and described.

Further, since a flowchart diagram illustrating an example of a procedure of the inertial navigation operation process (process of S40 in FIG. 14) in the second embodiment is the same as that in the first embodiment (FIG. 15), the flowchart will not be illustrated and described.

Further, since a flowchart diagram illustrating an example of a procedure of the running detection process (the process of S120 in FIG. 15) in the second embodiment is the same as that in the first embodiment (FIG. 16), the flowchart will not be illustrated and described.

Further, since a flowchart diagram illustrating an example of a procedure of the exercise analysis information generation process (the process of S50 in FIG. 14) in the second embodiment is the same as that in the first embodiment (FIG. 17), the flowchart will not be illustrated and described.

### 2-4. Reporting Device

### 2-4-1. Configuration of the Reporting Device

Since an example of a configuration of the reporting device 3 in the second embodiment is the same as that in the first embodiment (FIG. 18), the example is not illustrated. In the reporting device 3 in the second embodiment, respective functions of the storage unit 130, the manipulation unit 150, the clocking unit 160, the display unit 170, the sound output unit 180, and the vibration unit 190 are the same as those in the first embodiment, description thereof will be omitted.

The communication unit 140 is a communication unit that performs data communication with the communication unit 40 of the exercise analysis device 2 (see FIG. 3), and performs, for example, a process of receiving a command (for example, measurement start/measurement end command) according to manipulation data from the processing unit 120 and transmitting the command to the communication unit 40 of the exercise analysis device 2, or a process of receiving the output information during running or the running result information transmitted from the communication unit 40 of the exercise analysis device 2 and sending the information to the processing unit 120.

The processing unit 120 includes, for example, a CPU, a DSP, and an ASIC, and executes a program stored in the storage unit 130 (recording medium) to perform various operation processes and control processes, similar to the first embodiment.

Further, in the second embodiment, the processing unit 120, for example, sets a target value of each exercise index based on the manipulation data received from the manipulation unit 150 prior to running of the user (prior to transmission of the measurement start command). Also, the processing unit 120 compares the value of each exercise index included in the output information during running with each target value, generates information on the exercise state in the running of the user according to a comparison result, and reports the information to the user via the sound output unit 180 or the vibration unit 190, similar to the first embodiment.

### 2-4-2. Procedure of the Process

Since a flowchart illustrating an example of a procedure of a reporting process performed by the processing unit 120 in the second embodiment is the same as that in the first embodiment (FIG. 20), the flowchart will not be illustrated and described. Also, in the present embodiment, the processing unit 120 may acquire the target value of each exercise index based on manipulation data from the manipulation unit 150 in S400 of FIG. 20.

### 2-5. Image Generation Device

### 2-5-1. Configuration of the Image Generation Device

FIG. 35 is a functional block diagram illustrating an example of a configuration of the image generation device 4A. As illustrated in FIG. 35, the image generation device 4A includes a processing unit 420, a storage unit 430, a communication unit 440, a manipulation unit 450, a communication unit 460, a display unit 470, and a sound output unit 480, similar to the exercise analysis device 2 in the first embodiment. However, in the image generation device 4A of the present embodiment, some of these components may be removed or changed, or other components may be added. Since respective functions of the display unit 470 and the sound output unit 480 are the same as those in the first embodiment, description thereof will be omitted.

The communication unit 440 is a communication unit that performs data communication with the communication unit 40 of the exercise analysis device 2 (see FIG. 3). The communication unit 440 performs, for example, a process of receiving the transmission request command for requesting transmission of the exercise analysis information designated according to the manipulation data (exercise analysis information included in the running data that is a registration target) from the processing unit 420, transmitting the transmission request command to the communication unit 40 of the exercise analysis device 2, receiving the exercise analysis information from the communication unit 40 of the exercise analysis device 2, and sending the exercise analysis information to the processing unit 420.

The communication unit 460 is a communication unit that performs data communication with the server 5, and performs, for example, a process of receiving running data that is a registration target from the processing unit 420 and transmitting the running data to the server 5 (running data registration process), and a process of receiving management information corresponding to manipulation data of registration, editing, and deletion of a user, and editing, deletion, and replacement of the running data from the processing unit 420 and transmitting the management information to the server 5.

The manipulation unit 450 performs a process of acquiring manipulation data from the user (manipulation data of registration, editing, and deletion of the user, and registration, editing, deletion, and replacement of the running data, or manipulation data for selecting the user who is an analysis target), and sending the manipulation data to processing unit 420. The manipulation unit 450 may be, for example, a touch panel display, a button, a key, or a microphone.

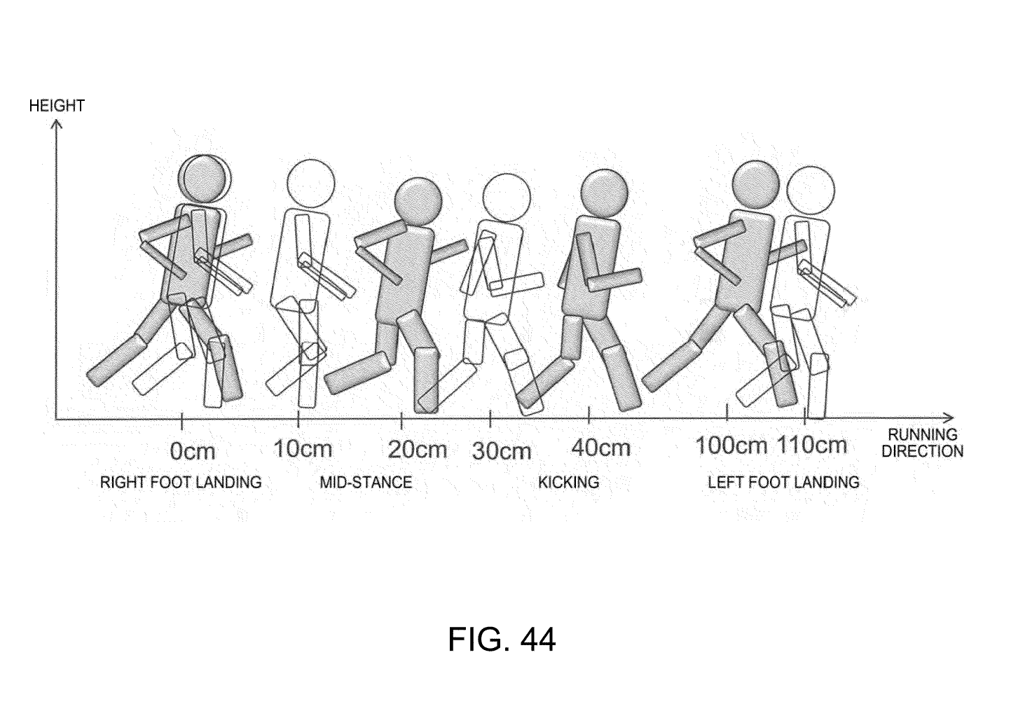

The storage unit 430 includes, for example, a recording medium that stores a program or data, such as a ROM, a flash ROM, a hard disk, or a memory card, or a RAM that is a work area of the processing unit 420. An image generation program 434 read by the processing unit 420, for executing the image generation process (see FIG. 44) is stored in the storage unit 430 (one of the recording media).

The processing unit 420 includes, for example, a CPU, a DSP, and an ASIC, and executes various programs stored in the storage unit 430 (recording medium) to perform various operation processes or control processes that are the same as those in the first embodiment.

In particular, in the present embodiment, the processing unit 420 executes the image generation program 434 stored in the storage unit 430 to function as an exercise analysis information acquisition unit 422 and an analysis information generation unit 424. However, the processing unit 420 may receive and execute the image generation program 434 stored in any storage device (recording medium) via a network or the like.

The motion analysis information acquisition unit 422 performs a process of acquiring exercise analysis information of the user at the time of running, which is generated using the measurement result of the inertial measurement unit (IMU) 10. For example, the exercise analysis information acquisition unit 422 may acquire the exercise analysis information (exercise analysis information generated by the exercise analysis device 2) that is the information on the analysis result of the exercise of the user who is an analysis target, from a database of the server 5 (or from the exercise analysis device 2). The exercise analysis information acquired by the exercise analysis information acquisition unit 422 is stored in the storage unit 430. In the present embodiment, the exercise analysis information acquired by the exercise analysis information acquisition unit 422 includes the values of various exercise indexes.

The image information generation unit 428 performs a process of generating the image information in which the exercise analysis information acquired by the exercise analysis information acquisition unit 422 is associated with the image data of the user object indicating the running of the user. For example, the image information generation unit 428 may generate image information including image data indicating the running state of the user who is an analysis target using the exercise analysis information acquired by the exercise analysis information acquisition unit 422. The image information generation unit 428, for example, may generate the image information using the exercise analysis information included in the running data selected by the user who is an analysis target selected in the manipulation data received from the manipulation unit 450. This image information may include two-dimensional image data or may include three-dimensional image data.

The image information generation unit 428 may generate image data indicating the running state of the user using the value of at least one exercise index included in the exercise analysis information acquired by the exercise analysis information acquisition unit 422. Further, the image information generation unit 428 may calculate a value of at least one exercise index using the exercise analysis information acquired by the exercise analysis information acquisition unit 422, and generate image data indicating the running state of the user using the values of the calculated exercise indexes.

Further, the image information generation unit 428 may generate the image information using the values of the various exercise indexes included in the exercise analysis information acquired by the exercise analysis information acquisition unit 422, and information regarding the posture angles (roll angle, pitch angle, and yaw angle).

Further, the image information generation unit 428 may generate comparison image data for comparison with the image data indicating the running state of the user, and generate image information including the image data indicating the running state of the user and the comparison image data. The image information generation unit 428, for example, may generate the comparison image data using values of various exercise indexes included in other running data (exercise analysis information) of the user who is an analysis target or values of various exercise indexes included in running data (exercise analysis information) of another user, or may generate the comparison image data using ideal values of various exercise indexes.

Further, the image information generation unit 428 may generate image information including image data indicating the running state at the feature point of the exercise of the user using the exercise analysis information acquired by the exercise analysis information acquisition unit 422.

The image information generation unit 428 may generate the image information including a plurality of pieces of image data indicating the running states at the multiple types of feature points of the exercise of the user using the exercise analysis information acquired by the exercise analysis information acquisition unit 422. For example, the image information generation unit 428 may generate the image information in which the plurality of pieces of image data are arranged side by side on a time axis or a space axis. Further, the image information generation unit 428 may generate a plurality of pieces of supplement image data for supplementing the plurality of pieces of image data on a time axis or a space axis, and generate image information including moving image data having the plurality of pieces of image data and the plurality of pieces of supplement image data.

In the present embodiment, the image information generation unit 428 generates image information in four modes that are selectable by manipulating the manipulation unit 450.

Mode 1 is a mode in which a time when the foot of the user who is an analysis target lands, a time of mid-stance, and a time of kicking (time of separation from ground) are three types of feature points, still images showing running of the user at the three types of feature points (images of the user object imitating a running state of the user) are displayed sequentially and repeatedly, or the user object is reproduced as a moving image. Any of the still image and the moving image to be displayed can be selected by manipulating the manipulation unit 450.

Mode 2 is a mode in which any one of images of a user object and an image of a comparison object are displayed to be superimposed at the three feature points for each of various exercise indexes of the user who is an analysis target.

Mode 3 is a mode in which the image of the user object at the three types of feature points and the image of the comparison object at the three types of feature points are displayed side-by-side on a time axis, like time-based continuous photos, or a moving image in which the user object and the comparison object move on the time axis is reproduced. Any of the time-based continuous photos and the moving image to be displayed can be selected by manipulating the manipulation unit 450.

Mode 4 is a mode in which the image of the user object at the three types of feature points and the image of the comparison object at the three types of feature points are displayed side-by-side on a spatial axis, like location-based continuous photos, or a moving image in which the user object and the comparison object move on the space axis is reproduced. Any of the location-based continuous photos and the moving image to be displayed can be selected by manipulating the manipulation unit 450.

In mode 1 to mode 4, the image information generation unit 428 repeatedly generates image data of three types of user objects indicating the running state at the three types of feature points (image data at the time of landing, the image data at the time of mid-stance, and image data at the time of kicking) in time series.

Also, in mode 1, the image information generation unit 428 displays the generated image data of the user object on the display unit 470. Alternatively, the image information generation unit 428 estimates a shape of the user object at any time between the two types of feature points from shapes of two types of user objects at any two types of consecutive feature points through linear supplement, generates image data of the user object, and reproduces a moving image.

Also, in mode 2 to mode 4, the image information generation unit 428 also repeatedly generates image data of three types of comparison objects at the three types of feature points (image data at the time of landing, image data of mid-stance, and image data at the time of kicking) in time series.

Also, in mode 2, the image information generation unit 428 generates image data in which the user object and the comparison object of any one of the three types are superimposed, for each of the various exercise indexes, and displays the image data on the display unit 470.

Further, in mode 3, the image information generation unit 428 generates image data (time-based continuous photos) in which the three types of user objects are arranged in a place corresponding to a time difference at the three types of feature points on the time axis, and the three types of comparison objects are arranged in a place corresponding to a time difference at the three types of feature points on the time axis, and displays image data on the display unit 470. Alternatively, the image information generation unit 428 generates image data of the user object and image data of the comparison object in an arbitrary time between any two types of consecutive feature points, and reproduces a moving image in which the user object and the comparison object move on the time axis.

Further, in mode 4, the image information generation unit 428 generates image data (location-based continuous photos) in which the three types of user objects are arranged in a place corresponding to a difference between the distances in the running direction at the three types of feature points on the axis in the running direction, and the three types of comparison objects are arranged in a place corresponding to a difference between the distances in the running direction at the three types of feature points on the axis in the running direction, and displays image data on the display unit 470. Alternatively, the image information generation unit 428 generates image data of the user object and image data of the comparison object in arbitrary distance in the running direction between any two types of consecutive feature points, and reproduces a moving image in which the user object and the comparison object move on the axis in the running direction.

### 2-5-2. Method of Generating Image Data at the Time of Landing

The image information generation unit 428, for example, can generate image data indicating a running state at the time of landing, using the posture angle (roll angle, pitch angle, and yaw angle) at the time of landing of the user who is an analysis target, and the value of the directly-under landing (directly-under landing rate 3) that is an exercise index. The posture angle or the value of directly-under landing is included in the exercise analysis information acquired by the exercise analysis information acquisition unit 422.

The image information generation unit 428, for example, detects landing at a timing at which the acceleration in the vertical direction included in the exercise analysis information changes from a positive value to a negative value, and selects a posture angle at the time of landing and a value of directly-under landing from the exercise analysis information. The image information generation unit 428 can identify whether the detected landing is landing of the right foot or landing of the left foot using the right and left leg flag included in the exercise analysis information.

Also, the image information generation unit 428 determines a slope of the torso of the user from the posture angle (roll angle, pitch angle, and yaw angle) at the time of landing. Further, the image information generation unit 428 determines a distance from a center of gravity to a landing leg from the value of directly-under landing. Further, the image information generation unit 428 determines the position of a pulling leg (rear leg) from the yaw angle at the time of landing. Further, the image information generation unit 428 determines the position or the angle of a head and an arm according to the determined information.

FIGS. 36A, 36B, and 36C illustrate examples of image data indicating the running state when the user who is an analysis target lands with the right foot, and show image data of images when the user who is an analysis target is viewed from a right side, a back, and a top, respectively. In the examples of FIGS. 36A, 36B, and 36C, the roll angle, the pitch angle, and the yaw angle at the time of landing are 3°, 0°, and 20°, and the directly-under landing is 30 cm.

Further, the image information generation unit 428 generates the image data for comparison, similar to the image data of the user who is an analysis target, using the posture angle (roll angle, pitch angle, and yaw angle) at the time of landing of the user who is a comparison target, and the value of the directly-under landing (directly-under landing rate 3) or using ideal values thereof.

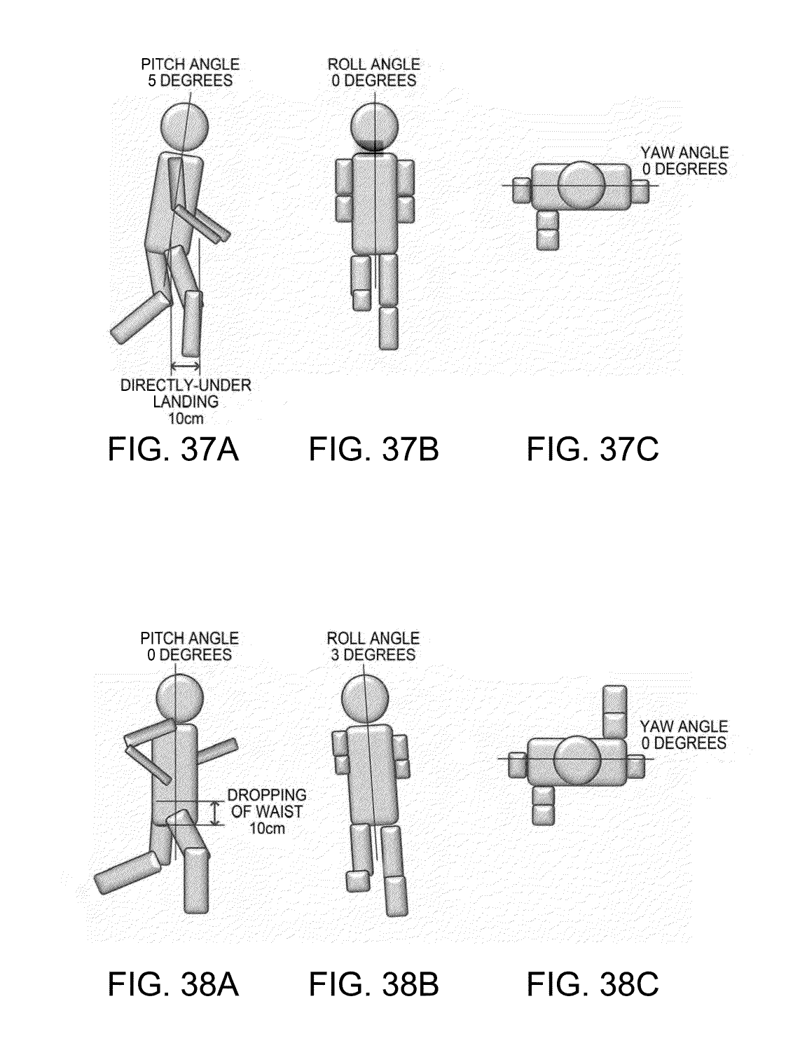

FIGS. 37A, 37B, and 37C illustrate examples of image data for comparison with the image data of the user who is an analysis target illustrated in FIGS. 36A, 36B, and 36C, and show image data of images when the user who is a comparison target is viewed from a right side, a back, and a top, respectively. In the examples of FIGS. 37A, 37B, and 37C, the roll angle, the pitch angle, and the yaw angle at the time of landing are 0°, 5°, and 0°, and the directly-under landing is 10 cm.

Also, FIGS. 36A, 36B, and 36C or FIGS. 37A, 37B, and 37C illustrate three-dimensional image data, the image information generation unit 428 may generate, for example, only two-dimensional image data of FIG. 36A or 37A.

### 2-5-3. Method of Generating Image Data of Mid-Stance

The image information generation unit 428, for example, can generate image data indicating a running state at the time of landing, using the posture angle (roll angle, pitch angle, and yaw angle) of mid-stance of the user who is an analysis target, and a value of dropping of the waist that is an exercise index. The value of this posture angle is included in the exercise analysis information acquired by the exercise analysis information acquisition unit 422, but the value of the dropping of the waist is not included in the exercise analysis information. The dropping of the waist is an exercise index calculated as a difference between a height of the waist at the time of landing and a height of the waist of mid-stance, and the image information generation unit 428 can calculate the value of the dropping of the waist using the value of the distance in the vertical direction in the exercise analysis information.

The image information generation unit 428 detects landing, detects, for example, mid-stance at a timing at which the acceleration in the vertical direction included in the exercise analysis information is maximized, and selects the posture angle and the distance in the vertical direction at the time of landing, and the distance in the vertical direction of the mid-stance from the exercise analysis information. The image information generation unit 428 can calculate a difference between the distance in the vertical direction at the time of landing and the distance in the vertical direction of mid-stance, and sets the difference as the value of the dropping of the waist.

Also, the image information generation unit 428 determines a slope of the torso of the user from the posture angle (roll angle, pitch angle, and yaw angle) of the mid-stance. Further, the image information generation unit 428 determines a bending state of a knee or a decrease state of the center of gravity from the value of the dropping of the waist. Further, the image information generation unit 428 determines the position of a pulling leg (rear leg) from the yaw angle at the time of landing. Further, the image information generation unit 428 determines the position or the angle of the head and the arm according to the determined information.

FIGS. 38A, 38B, and 38C illustrate examples of image data indicating the running state of the mid-stance when the right foot of the user who is an analysis target is grounded, and show image data of images when the user who is an analysis target is viewed from a right side, a back, and a top, respectively. In the examples of FIGS. 38A, 38B, and 38C, the roll angle, the pitch angle, and the yaw angle at the time of landing are 3°, 0°, and 0°, and the dropping of the waist is 10 cm.

Further, the image information generation unit 428 generates the image data for comparison, similar to the image data of the user who is an analysis target, using the posture angle (roll angle, pitch angle, and yaw angle) of the mid-stance of the user who is a comparison target, and the value of the dropping of the waist or using ideal values thereof.

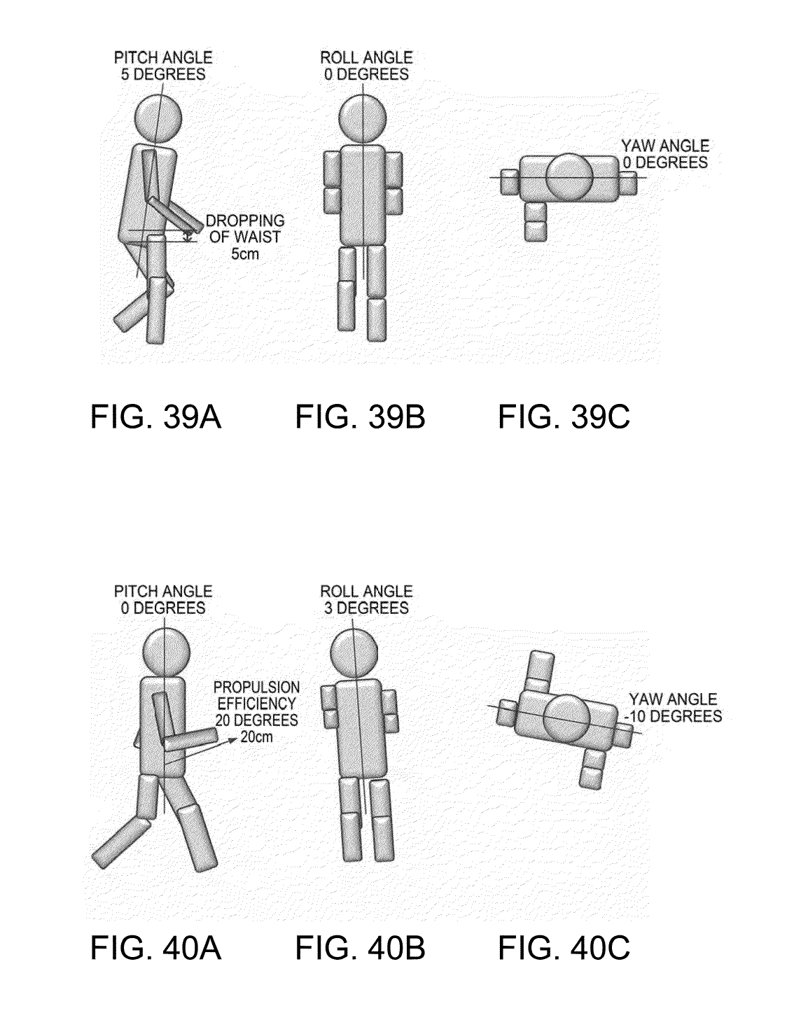

FIGS. 39A, 39B, and 39C illustrate examples of image data for comparison with the image data of the user who is an analysis target illustrated in FIGS. 38A, 38B, and 38C, and show image data of images when the user who is a comparison target is viewed from a right side, a back, and a top, respectively. In the examples of FIGS. 39A, 39B, and 39C, the roll angle, the pitch angle, and the yaw angle at the time of landing are 0°, 5°, and 0°, and dropping of the waist is 5 cm.

Also, FIGS. 38A, 38B, and 38C or FIGS. 39A, 39B, and 39C illustrate three-dimensional image, and the data image information generation unit 428 may generate, for example, only two-dimensional image data of FIG. 38A or 39A.

### 2-5-4. Method of Generating Image Data at the Time of Kicking

The image information generation unit 428, for example, can generate image data indicating a running state at the time of kicking, using the posture angle (roll angle, pitch angle, and yaw angle) at the time of kicking of the user who is an analysis target, and the value of the propulsion efficiency (propulsion efficiency 3) that is an exercise index. The posture angle or the value of propulsion efficiency is included in the exercise analysis information acquired by the exercise analysis information acquisition unit 422.

The image information generation unit 428 detects kicking at a timing at which the acceleration in the vertical direction included in the exercise analysis information changes from a negative value to a positive value, and selects the posture angle and the value of the propulsion efficiency at the time of kicking from the exercise analysis information.

Also, the image information generation unit 428 determines a slope of the torso of the user from the posture angle (roll angle, pitch angle, and yaw angle) at the time of kicking. Further, the image information generation unit 428 determines an angle of the kicking leg from the value of the propulsion efficiency. Further, the image information generation unit 428 determines the position of a front leg from the yaw angle at the time of kicking. Further, the image information generation unit 428 determines the position or the angle of a head and an arm according to the determined information.

FIGS. 40A, 40B, and 40C illustrate examples of image data indicating the running state when the user who is an analysis target kicks with the right foot, and show image data of images when the user who is an analysis target is viewed from a right side, a back, and a top, respectively. In the examples of FIGS. 40A, 40B, and 40C, the roll angle, the pitch angle, and the yaw angle at the time of landing are 3°, 0°, and −10°, and the propulsion efficiency is 20° and 20 cm.

Further, the image information generation unit 428 generates the image data for comparison, similar to the image data of the user who is an analysis target, using the posture angle (roll angle, pitch angle, and yaw angle) at the time of kicking of the user who is a comparison target, and the value of the propulsion efficiency or using ideal values thereof.

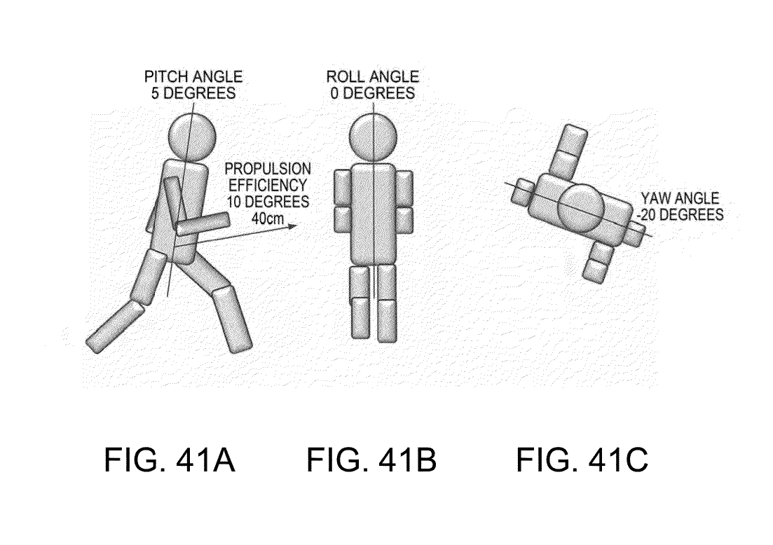

FIGS. 41A, 41B, and 41C illustrate examples of image data for comparison with the image data of the user who is an analysis target illustrated in FIGS. 40A, 40B, and 40C, and show image data of images when the user who is a comparison target is viewed from a right side, a back, and a top, respectively. In the examples of FIGS. 41A, 41B, and 41C, the roll angle, the pitch angle, and the yaw angle at the time of kicking are 0°, 5°, and −20°, and the propulsion efficiency is 10° and 40 cm.

Also, while FIGS. 40A, 40B, and 40C or FIGS. 41A, 41B, and 41C illustrate three-dimensional image data, the image information generation unit 428 may generate, for example, only two-dimensional image data of FIG. 40A or 41A.

### 2-5-5. Image Display Example

In mode 1, for example, images of the user object at the time of landing (FIG. 36A), the user object of mid-stance (FIG. 38A), and the user object at the time of kicking (FIG. 40A), viewed from the side, are sequentially and repeatedly displayed frame by frame in the same place. Further, when the manipulation unit 450 is manipulated, displaying is changed to displaying of frame by frame images (FIGS. 36B, 38B, and 40B) when the user object is viewed from the back, frame by frame images (FIGS. 36C, 38C, and 40C) when the user object is viewed from the top, or frame by frame images when the user object is viewed from any direction on a three-dimensional space. A shape of each user object at the time of landing, mid-stance, and kicking is changed every moment according to the data of the exercise analysis information of the user that is an analysis target. Alternatively, in mode 1, supplement is performed between frame-by-frame images, and a moving image in which the user object runs is displayed.

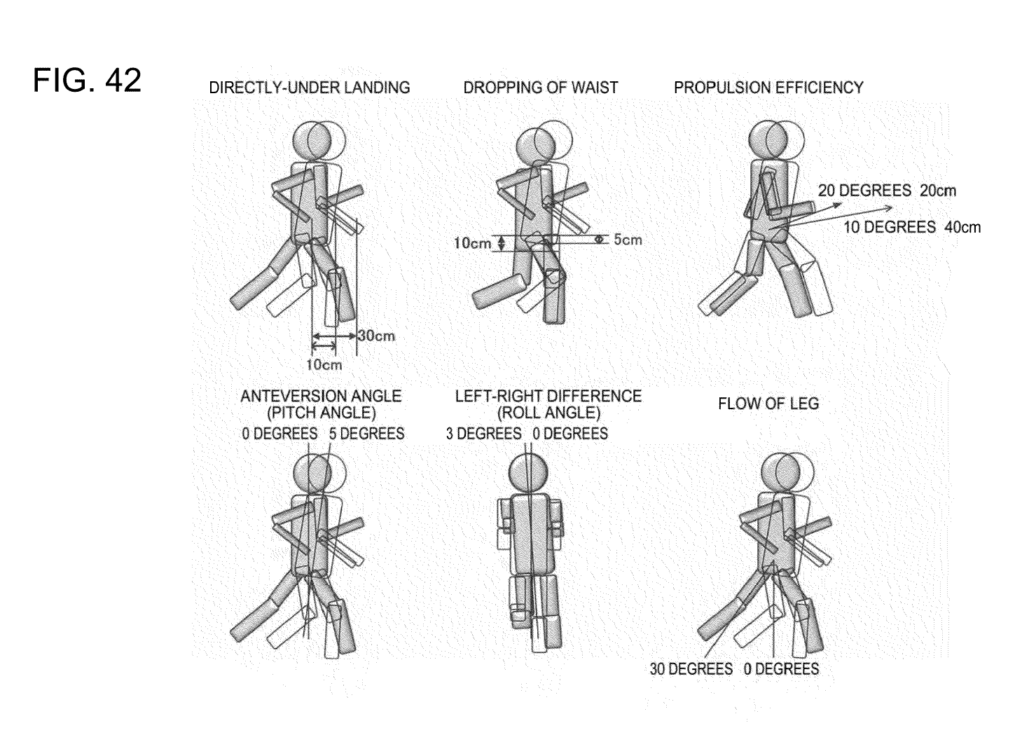

In mode 2, as illustrated in FIG. 42, an image in which the user object and the comparison object are superimposed, and numerical values are indicated to be easy understood for six exercise indexes of the directly-under landing, the dropping of the waist, the propulsion efficiency, the anteversion angle (pitch angle), left and right shake (roll angle), and the flow of the leg is displayed. In FIG. 42, a gray object is the user object, and a white object is the comparison object. For example, the user object (FIG. 36A) and the comparison object (FIG. 37A) at the time of landing as viewed from the side are used as the user object and the comparison object in the directly-under landing and the flow of the leg. The user object (FIG. 38A) and the comparison object (FIG. 39A) of mid-instance as viewed from the side are used as the user object and the comparison object in the dropping of the waist. The user object (FIG. 40A) and the comparison object (FIG. 41A) at the time of kicking viewed from the side are used as the user object and the comparison object in the propulsion efficiency. The user object (FIG. 36A) and the comparison object (FIG. 37A) at the time of landing, the user object (FIG. 38A) and the comparison object (FIG. 39A) of mid-instance, and the user object (FIG. 40A) and the comparison object (FIG. 41A) at the time of kicking, viewed from the side, are sequentially and repeatedly used as the user object and the comparison object in the anteversion angle (pitch angle). The user object (FIG. 36B) and the comparison object (FIG. 37B) at the time of landing, the user object (FIG. 38B) and the comparison object (FIG. 39B) of mid-instance, and the user object (FIG. 40B) and the comparison object (FIG. 41B) at the time of kicking, viewed from the back, are sequentially and repeatedly used as the user object and the comparison object in the left and right shake (roll angle). A shape of each user object is changed every moment according to the data of the exercise analysis information of the user that is an analysis target. When each comparison object is generated from the ideal values of the various exercise indexes, a shape of the comparison object is not changed, but when each comparison object is generated using the exercise analysis information of the user who is a comparison target, the shape of the comparison object is changed every moment according to the data of the exercise analysis information.

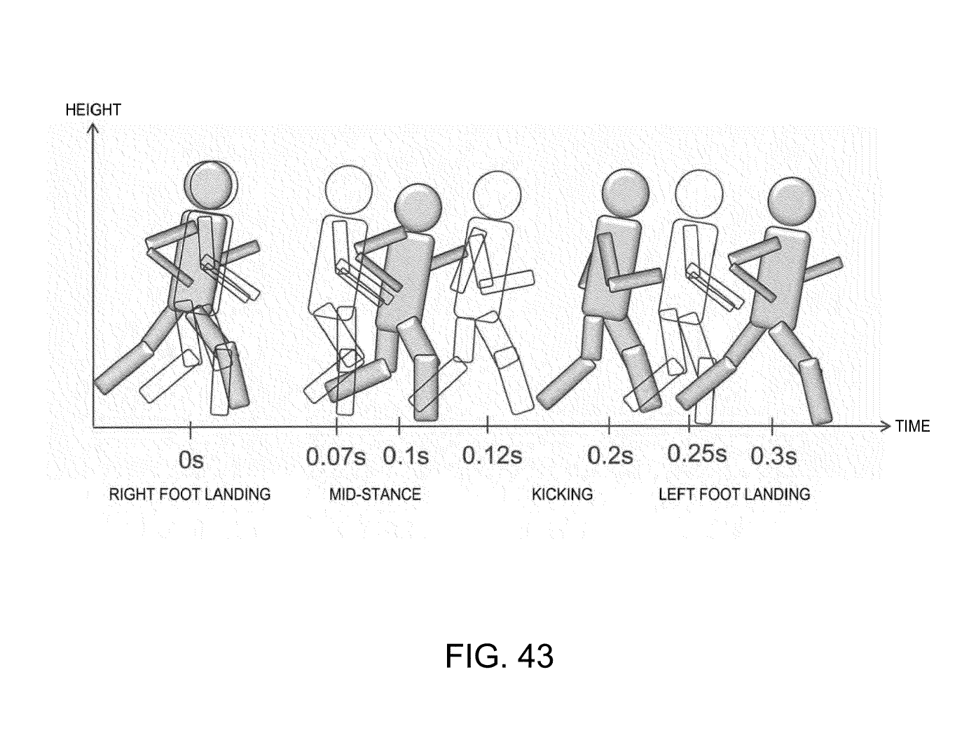

In mode 3, as illustrated in FIG. 43, for example, an image of time-based consecutive photos in which respective images of the user object (FIG. 36A) and the comparison object (FIG. 37A) at the time of landing, the user object (FIG. 38A) and the comparison object (FIG. 39A) of mid-stance, and the user object (FIG. 40A) and the comparison object (FIG. 41A) at the time of kicking, viewed from the side, are arranged on the time axis is displayed. In FIG. 43, a gray object is the user object, a white object is the comparison object, and the user object and the comparison object at the time of right foot landing are arranged in a position of 0 second on the time axis. Also, when respective user objects and respective comparison objects of mid-stance, at the time of kicking, and at the time of left foot landing are arranged in positions on the time axis according to a time taken from right foot landing. A shape or a position on the time axis of each user object is changed every moment according to the data of the exercise analysis information of the user that is an analysis target. When each comparison object is generated from the ideal values of the various exercise indexes, a shape or a position on the time axis of the comparison object is not changed, but when each comparison object is generated using the exercise analysis information of the user who is a comparison target, the shape or the position on the time axis of the comparison object is changed every moment according to the data of the exercise analysis information. Alternatively, in mode 3, a moving image in which the user object and the comparison object move on the time axis is displayed.

In mode 4, as illustrated in FIG. 44, for example, an image of position-based consecutive photos in which respective images of the user object (FIG. 36A) and the comparison object (FIG. 37A) at the time of landing, the user object (FIG. 38A) and the comparison object (FIG. 39A) of mid-stance, and the user object (FIG. 40A) and the comparison object (FIG. 41A) at the time of kicking, viewed from the side, are arranged on the axis in the running direction is displayed. In FIG. 44, a gray object is the user object, a white object is the comparison object, and the user object and the comparison object at the time of right foot landing are arranged in a position of 0 cm on the axis in the running direction. Also, when respective user objects and respective comparison objects of mid-stance, at the time of kicking, and at the time of left foot landing are arranged in positions on the axis in the running direction according to a movement distance in the running direction from right foot landing. A shape or a position on the axis in the running direction of each user object is changed every moment according to the data of the exercise analysis information of the user that is an analysis target. When each comparison object is generated from the ideal values of the various exercise indexes, a shape or a position on the axis in the running direction of the comparison object is not changed, but when each comparison object is generated using the exercise analysis information of the user who is a comparison target, the shape or the position on the axis in the running direction of the comparison object is changed every moment according to the data of the exercise analysis information. Alternatively, in mode 4, a moving image in which the user object and the comparison object move on the axis in the running direction is displayed.

### 2-5-6. Procedure of the Process

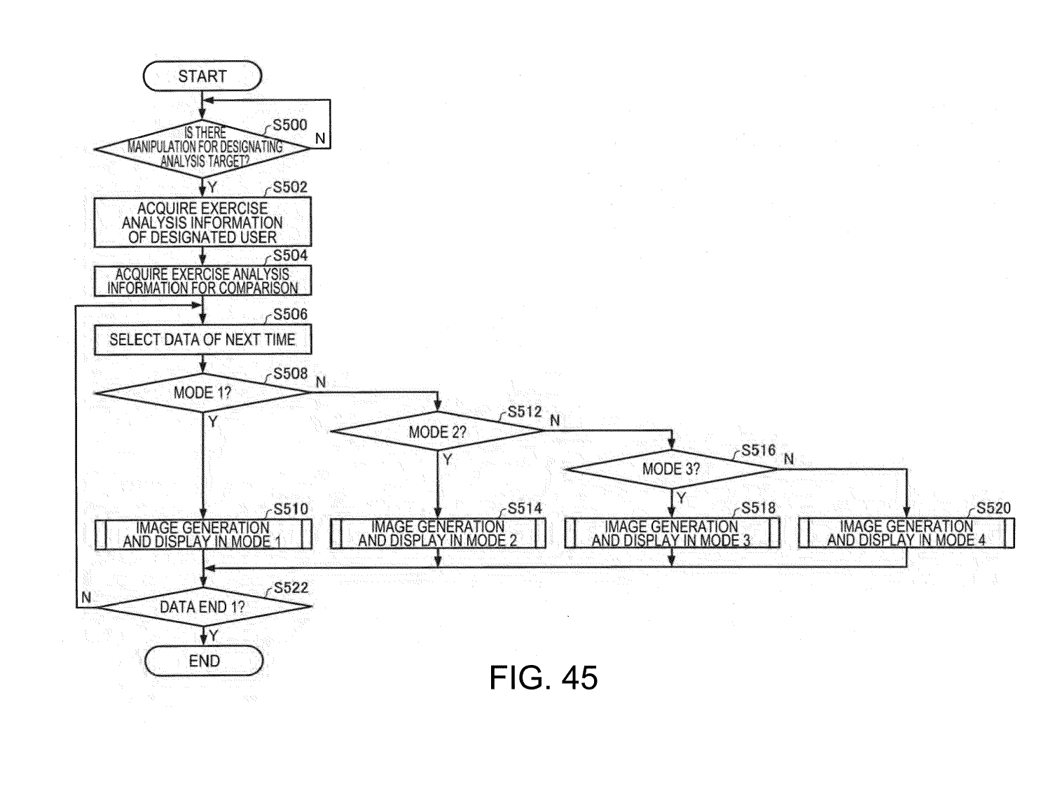

FIG. 45 is a flowchart diagram illustrating an example of a procedure of the image generation process performed by the processing unit 420 of the information analysis device 4A. The processing unit 420 of the information analysis device 4A (an example of a computer) executes the image generation program 434 stored in the storage unit 430 to execute, for example, the image generation process in the procedure of the flowchart in FIG. 45.

First, the processing unit 420 waits until the processing unit 420 acquires manipulation data for designating the analysis target (N in S500). When the processing unit 420 acquires the manipulation data for designating the analysis target (Y in S500), the processing unit 420 acquires exercise analysis information (specifically, running data) in the designated running of the user (user that is an analysis target) designated in the manipulation data from the database of the server 5 via the communication unit 460, and stores the exercise analysis information in the storage unit 430 (S502).

Then, the processing unit 420 acquires the exercise analysis information for comparison (for example, running data of the user who is a comparison target) from the database of the server 5 via the communication unit 460, and stores the exercise analysis information for comparison in the storage unit 430 (S504). When the image data for comparison is generated using an ideal value of each exercise index determined in advance, the processing unit 420 may not perform the process of S504.

Then, the processing unit 420 selects data (the user data and the comparison use data) of the next time (initial time) from each of the exercise analysis information (running data) acquired in S502 and the exercise analysis information (running data) acquired in S504 (S506).

Also, when mode 1 is selected (Y in S508), the processing unit 420 performs an image generation and display process of mode 1 (S510). An example of a procedure of this image generation and display process of mode 1 will be described below.

Further, when mode 2 is selected (N in S508 and Y in S512), the processing unit 420 performs an image generation and display process of mode 2 (S514). An example of a procedure of this image generation and display process of mode 2 will be described below.

Further, when mode 3 is selected (N in S512 and Y in S516), the processing unit 420 performs an image generation and display process in mode 3 (S518). An example of a procedure of this image generation and display process in mode 3 will be described below.

Further, when mode 4 is selected (N in S516), the processing unit 420 performs an image generation and display process of mode 4 (S520). An example of a procedure of this image generation and display process of mode 4 will be described below.

Also, when the processing unit 420 does not acquire manipulation data for image generation end (N in S522), the processing unit 420 selects data of the next time from each of the exercise analysis information acquired in S502 and the exercise analysis information acquired in S504 (S506), and performs any one of S510, S514, S518, and S520 again according to the selected mode. Further, when the processing unit 420 acquires the manipulation data for image generation end (Y in S522), and ends the image generation process.

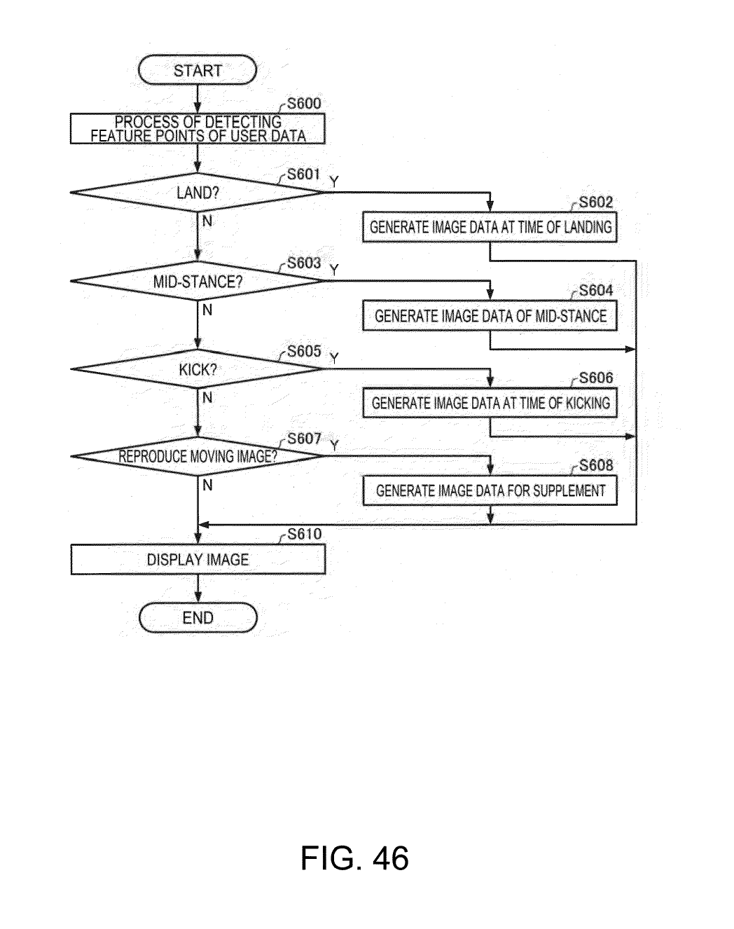

FIG. 46 is a flowchart diagram illustrating an example of a procedure of the image generation and display process in mode 1 (the process of S510 of FIG. 45). The processing unit 20 (image information generation unit 428) performs, for example, the image generation and display process in mode 1 in the procedure of the flowchart of FIG. 46.

First, the processing unit 420 performs a process of detecting feature points (landing, mid-stance, and kicking) using the user data selected in S506 of FIG. 45 (for example, the value of the acceleration in the vertical direction) (S600). When the processing unit 420 detects landing (Y in S601), the processing unit 420 generates image data at the time of landing (user object at the time of landing) (S602).

Further, when the processing unit 420 detects the mid-stance (N in S601 and Y in S603), the processing unit 420 generates image data of mid-stance (user object of the mid-stance) (S604).

Further, when the processing unit 420 detects kicking (N in S603 and Y in S605), the processing unit 420 generates image data of kicking (user object at the time of kicking) (S606).

Further, when the processing unit 420 does not detect any of landing, mid-stance, and kicking (N in S605), the processing unit 420 generates image data for supplement (user object for supplement) (S608) when moving image reproduction is selected (Y in S607), and does not perform the process in S608 when the moving image reproduction is not selected (N in S607).

Then, the processing unit 420 displays an image corresponding to the image data (user object) generated in S602, S604, S606, and S608 on the display unit 470 (S610), and ends the image generation and display process in mode 1 at the time. Also, when the processing unit 420 does not generate the image data in any of S602, S604, S606, and S608, the processing unit 420 continues to display the current image on the display unit 470 in S610, and ends the image generation and display process in mode 1 at the time.

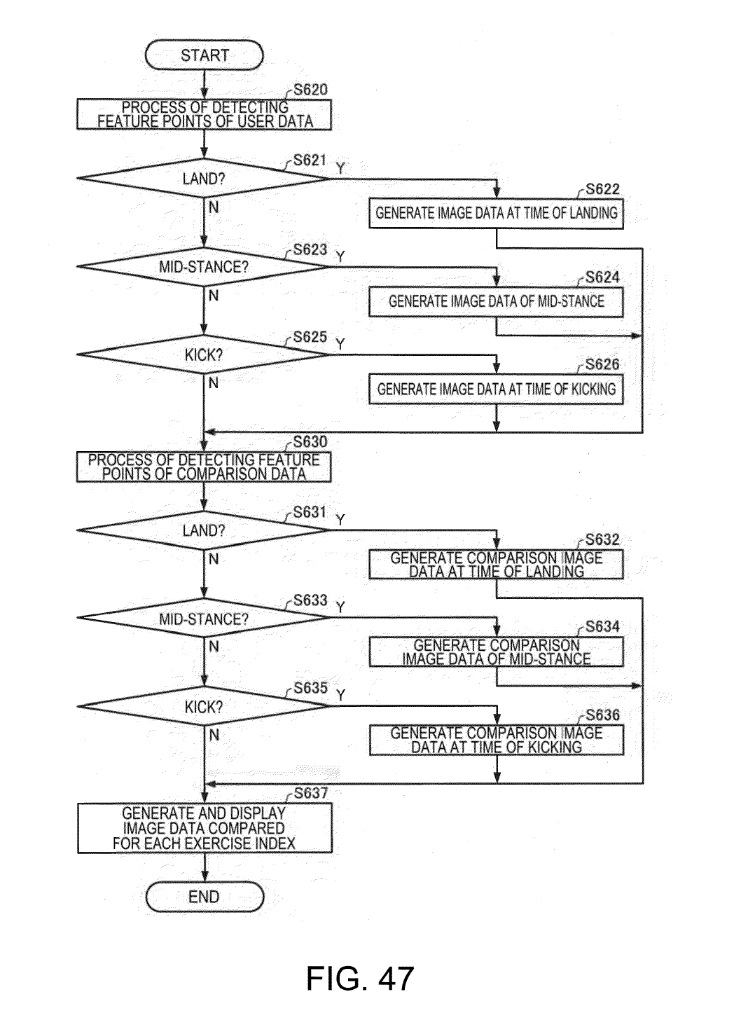

FIG. 47 is a flowchart diagram illustrating an example of a procedure of the image generation and display process in mode 2 (the process of S514 of FIG. 45). The processing unit 20 (image information generation unit 428) performs, for example, the image generation and display process in mode 2 in the procedure of the flowchart of FIG. 47.

First, the processing unit 420 performs the same process as S600 to S606 in the image generation and display process in the first mode (FIG. 46). When the processing unit 420 detects landing, mid-stance, or kicking, the processing unit 420 generates image data thereof (user object) (S620 to S626).

Then, the processing unit 420 performs a process of detecting feature points (landing, mid-stance, and kicking) using the comparison data (for example, the value of the acceleration in the vertical direction) selected in S506 of FIG. 45 (S630). When the processing unit 420 detects landing (Y in S631), the processing unit 420 generates comparison image data at the time of landing (comparison object at the time of landing) (S632).

Further, when the processing unit 420 detects mid-stance (N in S631 and Y in S633), the processing unit 420 generates comparison image data of mid-stance (comparison object at mid-stance) (S634).

Further, when the processing unit 420 detects kicking (N in S633 and Y in S635), the processing unit 420 generates comparison image data of kicking (comparison object at the time of kicking) (S636).

Also, the processing unit 420 generates image data in which the user object and the comparison object are compared for each exercise index using the image data (user object) generated in S622, S624, and S626, or the image data (comparison object) generated in S632, S634, and S636, displays an image corresponding to the image data on the display unit 470 (S637), and ends the image generation and display process in mode 2 at the time. Also, when the processing unit 420 does not generate the image data in any of S622, S624, S626, S632, S634, and S636, the processing unit 420 continues to display the current image on the display unit 470 in S637 and ends the image generation and display process in mode 2 at the time.

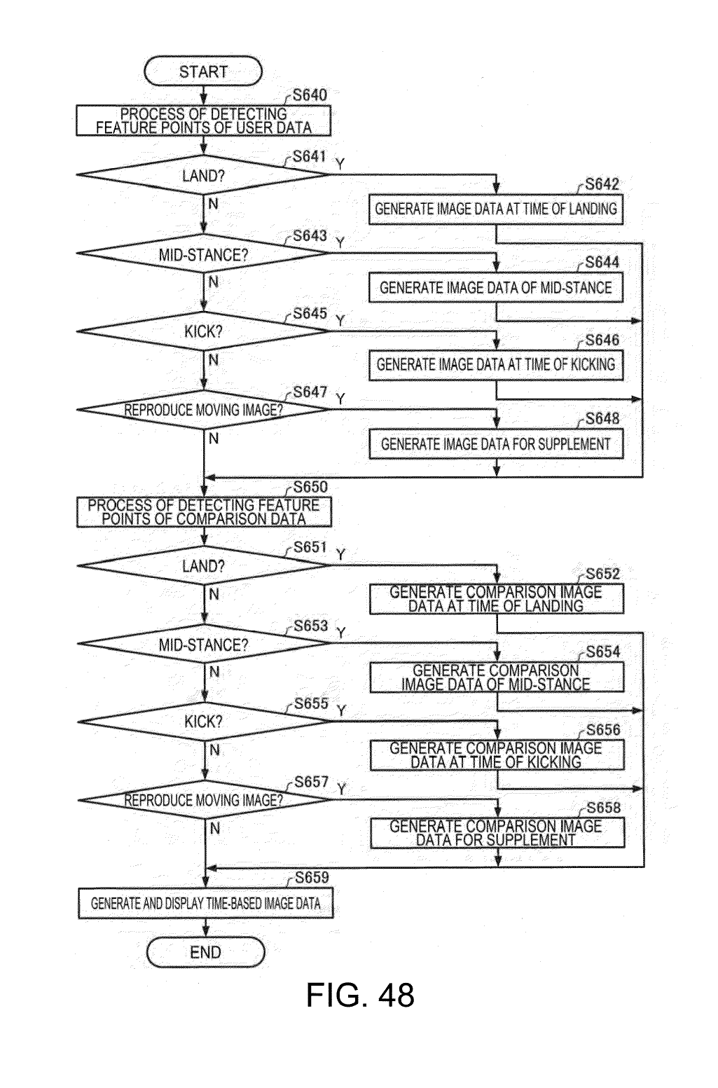

FIG. 48 is a flowchart diagram illustrating an example of a procedure of the image generation and display process in mode 3 (process of S518 in FIG. 45). The processing unit 20 (image information generation unit 428) executes the image generation and display process in mode 3, for example, in the procedure of the flowchart of FIG. 48.

First, the processing unit 420 performs the same process as S600 to S608 of the image generation and display process in the first mode (FIG. 46). When the processing unit 420 detects landing, mid-stance, or kicking, the processing unit 420 generates image data (user object) thereof, and when the processing unit 420 does not detect any of landing, mid-stance, or kicking, the processing unit 420 generates image data for complement (user object) if moving image reproduction is selected (S640 to S648).

Then, the processing unit 420 performs the same process as S630 to S636 in the image generation and display process in the second mode (FIG. 47). When the processing unit 420 detects landing, mid-stance, or kicking, the processing unit 420 generates comparison image data thereof (comparison user object) (S650 to S656).

Further, when the processing unit 420 does not detect any of landing, mid-stance, and kicking (N in S655), the processing unit 420 generates the comparison image data for supplement (comparison object for supplement) (S658) if the moving image reproduction is selected (Y in S657), and does not perform the process in S658 if moving image reproduction is not selected (N in S657).

Also, the processing unit 420 generates time-based image data using the image data (user object) generated in S642, S644, S646, and S648 or the image data (comparison object) generated in S652, S654, S656, and S658, displays an image corresponding to the time-based image data on the display unit 470 (S659), and ends the image generation and display process in mode 3 at the time. When the processing unit 420 does not generate the image data in any of S642, S644, S646, S648, S652, S654, S656, and S658, the processing unit 420 continues to display the current image on the display unit 470 in S659 and ends the image generation and display process in mode 3 at the time.

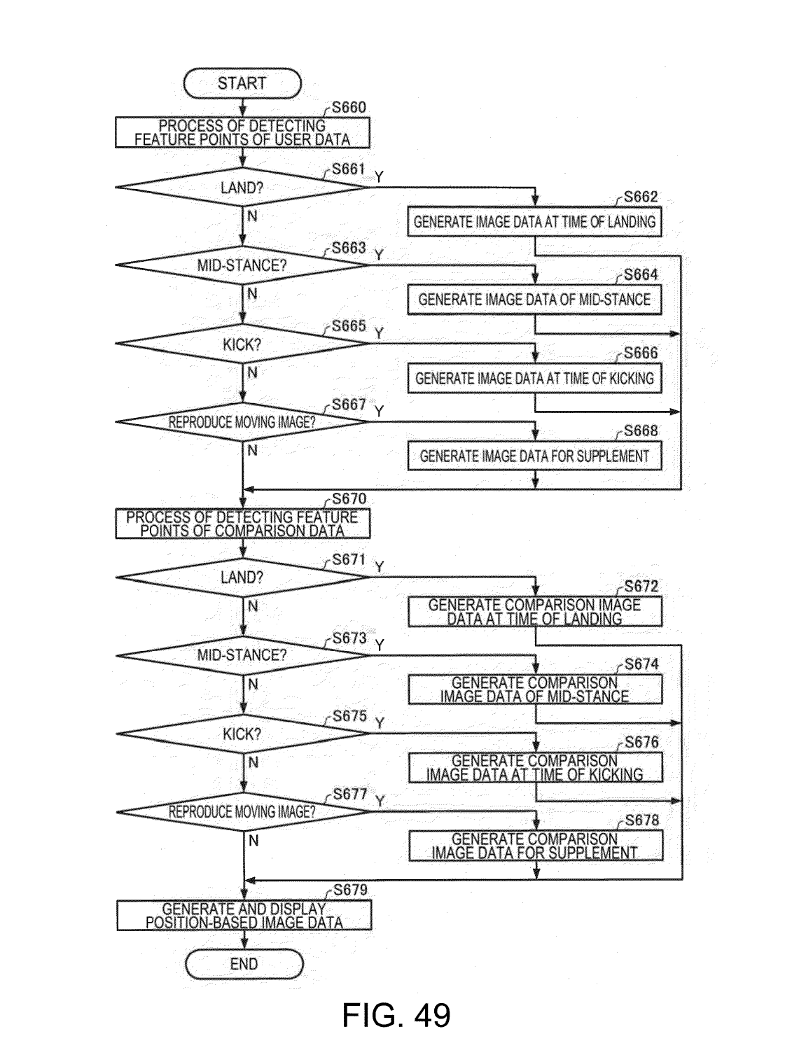

FIG. 49 is a flowchart diagram illustrating an example of a procedure of the image generation and display process in mode 4 (the process of S522 in FIG. 45). The processing unit 20 (image information generation unit 428) performs, for example, the image generation and display process in mode 4 in the procedure of the flowchart in FIG. 49.

First, the processing unit 420 performs the same process as S640 to S648 of the image generation and display process in the third mode (FIG. 48). When the processing unit 420 detects landing, mid-stance, or kicking, the processing unit 420 generates image data (user object) thereof, and when the processing unit 420 does not detect any of landing, mid-stance, or kicking, the processing unit 420 generates image data for complement (user object) if moving image reproduction is selected (S660 to S668).

Then, the processing unit 420 performs the same process as S650 to S658 of the image generation and display process in the third mode (FIG. 48). When the processing unit 420 detects landing, mid-stance, or kicking, the processing unit 420 generates comparison image data (comparison user object) thereof, and when the processing unit 420 does not detect any of landing, mid-stance, or kicking, the processing unit 420 generates image data for complement (user object) when moving image reproduction is selected (S670 to S678).

Also, the processing unit 420 generates position-based image data using the image data (user object) generated in S662, S664, S666, and S668 or the image data (comparison object) generated in S672, S674, S676, and S678, displays an image corresponding to the position-based image data on the display unit 470 (S679), and ends the image generation and display process in mode 4 at the time. Also, when the processing unit 420 does not generate the image data in any of S662, S664, S666, S668, S672, S674, S676, and S678, the processing unit 420 continues to display the current image on the display unit 470 in S679 and ends the image generation and display process in mode 4 at the time.

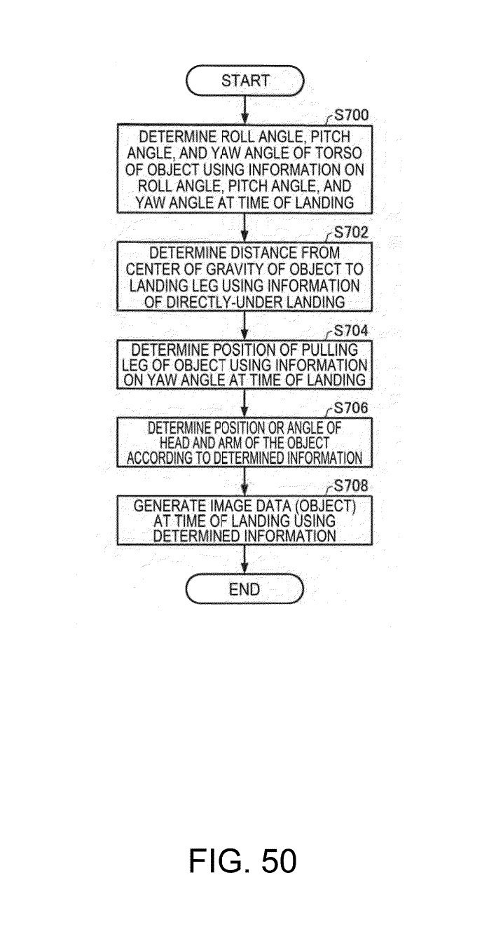

FIG. 50 is a flowchart diagram illustrating an example of a procedure of a process of generating the image data (user object or comparison object) at the time of landing (process in S602 of FIG. 46, process of S622 and S632 in FIG. 47, process of S642 and S652 in FIG. 48, and process of S662 and S672 in FIG. 49). The processing unit 20 (image information generation unit 428) executes, for example, a process of generating image data at the time of landing in the procedure of the flowchart of FIG. 50.

First, the processing unit 420 determines the roll angle, the pitch angle, and the yaw angle of the torso of the object (user object or comparison object) using information on the roll angle, the pitch angle, and the yaw angle at the time of landing (S700).

Then, the processing unit 420 determines the distance from the center of gravity of the object to the landing leg using the information of the directly-under landing (S702).

The processing unit 420 then determines the location of the pulling leg (rear leg) of the object using the information on the yaw angle at the time of landing (S704).

The processing unit 420 then determines the position or the angle of the head and the arm of the object according to the information determined in S700, S702, and S704 (S706).

Finally, the processing unit 420 generates image data (user object or comparison object) at the time of landing using the information determined in S700, S702, S704, and S706 (S708), and ends the process of generating the image data at the time of landing.

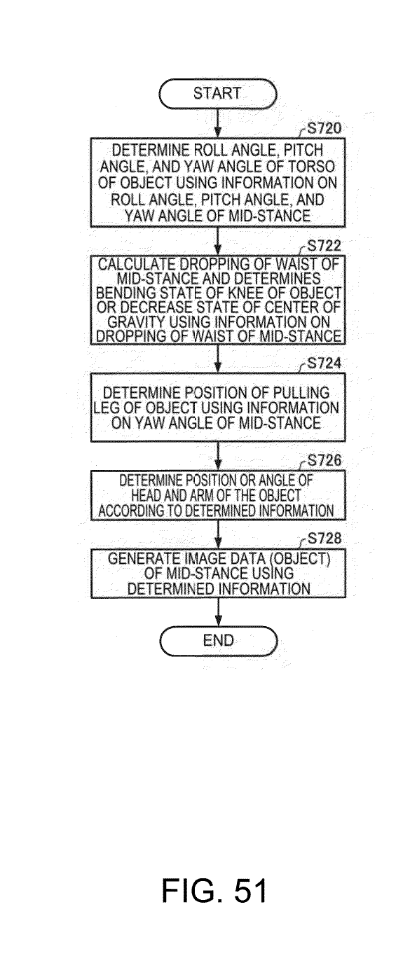

FIG. 51 is a flowchart diagram illustrating an example of a procedure of a process of generating the image data (user object or comparison object) of the mid-stance (process in S604 of FIG. 46, process of S624 and S634 in FIG. 47, process of S644 and S654 in FIG. 48, and process of S664 and S674 in FIG. 49). The processing unit 20 (image information generation unit 428) executes, for example, a process of generating image data of mid-stance in the procedure of the flowchart of FIG. 51.

First, the processing unit 420 determines the roll angle, the pitch angle, and the yaw angle of the torso of the object (user object or comparison object) using information on the roll angle, the pitch angle, and the yaw angle of the mid-stance (S720).

Then, the processing unit 420 calculates the dropping of the waist of the mid-stance, and determines a bending state of a knee of the object or a decrease state of the center of gravity using information on the dropping of the waist (S722).

The processing unit 420 then determines the location of the pulling leg (rear leg) of the object using the information on the yaw angle of the mid-stance (S724).

The processing unit 420 then determines the position or the angle of the head and the arm of the object according to the information determined in S720, S722, and S724 (S726).

Finally, the processing unit 420 generates image data (user object or comparison object) of mid-stance using the information determined in S720, S722, S724, and S726 (S728), and ends the process of generating the image data of the mid-stance.

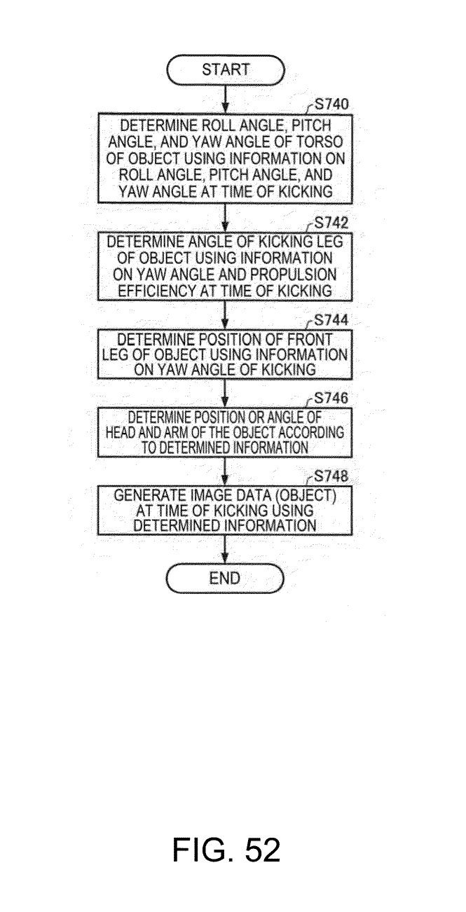

FIG. 52 is a flowchart diagram illustrating an example of a procedure of a process of generating the image data (user object or comparison object) at the time of kicking (process in S606 of FIG. 46, process of S626 and S636 in FIG. 47, process of S646 and S656 in FIG. 48, and process of S666 and S676 in FIG. 49). The processing unit 20 (image information generation unit 428) executes, for example, a process of generating image data at the time of kicking in the procedure of the flowchart of FIG. 52.

First, the processing unit 420 determines the roll angle, the pitch angle, and the yaw angle of the torso of the object (user object or comparison object) using information on the roll angle, the pitch angle, and the yaw angle at the time of kicking (S740).

The processing unit 420 then determines the angle of the kicking leg of the object using the information on the yaw angle and the propulsion efficiency at the time of kicking (S742).

The processing unit 420 then determines the position of the front leg of the object using the information on the yaw angle of kicking (S744).

The processing unit 420 then determines the position or the angle of the head and the arm of the object according to the information determined in S740, S742, and S744 (S746).

Finally, the processing unit 420 generates image data (user object or comparison object) at the time of kicking using the information determined in S740, S742, S744, and S746 (S748), and ends the process of generating the image data at the time of kicking.

### 2-6. Effects

According to the second embodiment, since the inertial measurement unit 10 can detect a fine motion of the user using the 3-axis acceleration sensor 12 and the 3-axis angular speed sensor 14, the exercise analysis device 2 can perform the inertial navigation operation using the detection result of the inertial measurement unit 10 during running of the user and can accurately calculate the values of various exercise indexes related to the running capability using the result of the inertial navigation operation. Thus, the image generation device 4A can generate the image information for accurately reproducing the state of the portion closely related to the running capability using the values of various exercise indexes calculated by the exercise analysis device 2. Therefore, the user can visually and clearly recognize the state of the most desired portion using the image information although the user does not accurately recognize the motion of the entire body.

In particular, in the second embodiment, since the exercise analysis device 2 (inertial measurement unit 10) is amounted on a torso portion (for example, waist) of the user, the image generation device 4A can generate image information for accurately reproducing a torso state closely related to the running capability and accurately reproducing a state of the leg from the state of the torso.

Further, according to the second embodiment, since the image generation device 4A sequentially and repeatedly displays the user object at three feature points landing, mid-stance, and kicking in mode 1, the user can recognize the running state during grounding in detail.

Further, according to the second embodiment, since the image generation device 4A displays the user object and the comparison object in a superimposing manner for various exercise indexes closely related to the running capability in mode 2, the user can easily perform the comparison and objectively evaluate the running capability of the user.

Further, according to the second embodiment, since the image generation device 4A displays the user object and the comparison object at three feature points of landing, mid-stance, and kicking side by side on the time axis in mode 3, the user can easily perform both comparison of the running state for each feature point and comparison of the time difference, and can evaluate the running capability of the user more accurately.

Further, according to the second embodiment, since the image generation device 4A displays the user object and the comparison object at three feature points of landing, mid-stance, and kicking side by side on the running direction axis in mode 4, the user can easily perform both comparison of the running state for each feature point and comparison of the movement distance, and can evaluate the running capability of the user more accurately.

### 3. Third Embodiment

In a third embodiment, the same components as those in the first embodiment or the second embodiment are denoted with the same reference numerals, and description thereof will be omitted or simplified. Different content from those in the first embodiment and the second embodiment will be described.

### 3-1. Configuration of Information Display System

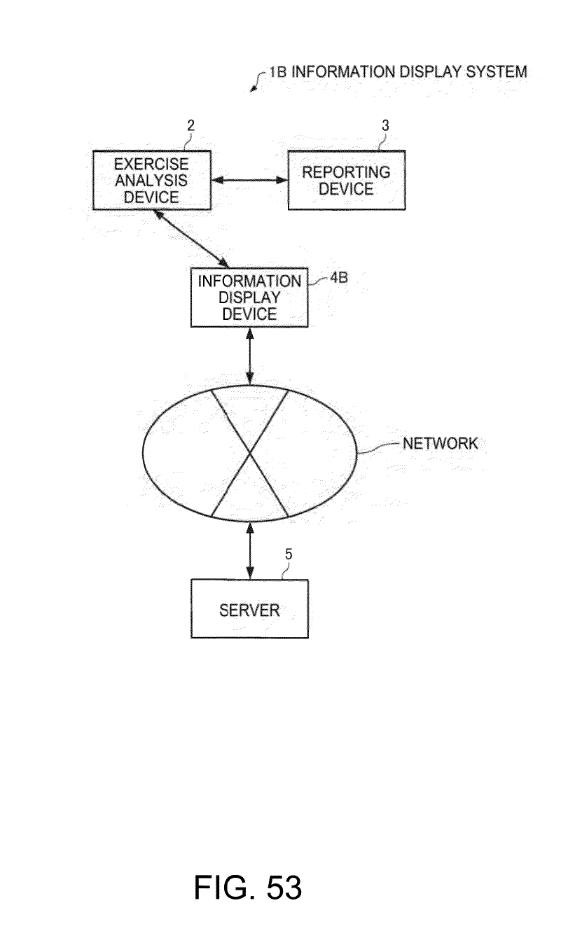

Hereinafter, an information display system that analyzes exercise in running (including walking) of a user will be described by way of example, but an information display system of a third embodiment may be an information display system that analyzes exercise other than running. FIG. 53 is a diagram illustrating an example of a configuration of an information display system 1B of the third embodiment. As illustrated in FIG. 53, the information display system 1B of the third embodiment includes an exercise analysis device 2, a reporting device 3, and an information display device 4B. The exercise analysis device 2 is a device that analyzes exercise during running of the user, and the reporting device 3 is a device that notifies the user of information on a state during running of the user or a running result, similar to the first or second embodiment. The information display device 4B is a device that analyzes and presents the running result after running of the user ends. In the third embodiment, as illustrated in FIG. 2, the exercise analysis device 2 includes an inertial measurement unit (IMU) 10, and is mounted to a torso portion (for example, a right waist, a left waist, or a central portion of a waist) of the user so that one detection axis (hereinafter referred to as a z axis) of the inertial measurement unit (IMU) 10 substantially matches a gravitational acceleration direction (vertically downward) in a state in which the user is at rest, similar to the first or second embodiment. Further, the reporting device 3 is a wrist type (wristwatch type) portable information device, and is mounted on, for example, the wrist of the user. However, the reporting device 3 may be a portable information device, such as a head mount display (HMD) or a smartphone.

The user operates the reporting device 3 at the time of running start to instruct the exercise analysis device 2 to start measurement (inertial navigation operation process and exercise analysis process to be described below), and operates the reporting device 3 at the time of running end to instruct to end the measurement in the exercise analysis device 2, similar to the first and second embodiments. The reporting device 3 transmits a command for instructing start or end of the measurement to the exercise analysis device 2 in response to the operation of the user.

Similar to the first or second embodiment, when the exercise analysis device 2 receives the measurement start command, the exercise analysis device 2 starts the measurement using an inertial measurement unit (IMU) 10, calculates values for various exercise indexes which are indexes regarding running capability (an example of exercise capability) of the user using a measurement result, and generates exercise analysis information including the values of the various exercise indexes as information on the analysis result of the running exercise of the user. The exercise analysis device 2 generates information to be output during running of the user (output information during running) using the generated exercise analysis information, and transmits the information to the reporting device 3. The reporting device 3 receives the output information during running from the exercise analysis device 2, compares the values of various exercise indexes included in the output information during running with respective previously set reference values, and reports goodness or badness of the exercise indexes to the user through sound or vibration. Thus, the user can run while recognizing the goodness or badness of each exercise index.

Further, similar to the first or second embodiment, when the exercise analysis device 2 receives the measurement end command, the exercise analysis device 2 ends the measurement of the inertial measurement unit (IMU) 10, generates user running result information (running result information: running distance and running speed), and transmits the user running result information to the reporting device 3. The reporting device 3 receives the running result information from the exercise analysis device 2, and notifies the user the running result information as a text or an image. Accordingly, the user can recognize the running result information immediately after the running end.

Also, data communication between the exercise analysis device 2 and the reporting device 3 may be wireless communication or may be wired communication.

Further, in the third embodiment, the exercise analysis system 1B includes a server 5 connected to a network, such as the Internet or local area network (LAN), as illustrated in FIG. 53, similar to the first or second embodiment. An information display device 4B is, for example, an information device such as a personal computer or a smart phone, and can perform data communication with the server 5 over the network. The information display device 4B acquires the exercise analysis information in past running of the user from the exercise analysis device 2, and transmits the exercise analysis information to the server 5 over the network. However, a device different from the information display device 4B may acquire the exercise analysis information from the exercise analysis device 2 and transmit the exercise analysis information to the server 5 or the exercise analysis device 2 may directly transmit the exercise analysis information to the server 5. The server 5 receives this exercise analysis information and stores the exercise analysis information in a database built in a storage unit (not illustrated). In the present embodiment, a plurality of users wear the same or different exercise analysis devices 2 and perform running, and the exercise analysis information of each user is stored in the database of the server 5.

The information display device 4B displays running state information that is information on at least one of the running speed and the running environment of the user, and the index regarding the running of the user calculated using the measurement result of the inertial measurement unit (IMU) 10 (detection result of the inertial sensor) in association with each other. Specifically, the information display device 4B acquires the exercise analysis information of the user from the database of the server 5 over the network, and displays the running state information and the index regarding running of the user, using the running state information included in the acquired exercise analysis information and the values of various exercise indexes, on the display unit (not illustrated in FIG. 53) in association with each other.

In the information display system 1B, the exercise analysis device 2, the reporting device 3, and the information display device 4B may be separately provided, the exercise analysis device 2 and the reporting device 3 may be integrally provided and the information display device 4B may be separately provided, the reporting device 3 and the information display device 4B may be integrally provided and the exercise analysis device 2 may be separately provided, the exercise analysis device 2 and the information display device 4B may be integrally provided and the reporting device 3 may be separately provided, or the exercise analysis device 2, the reporting device 3, and the information display device 4B may be integrally provided. The exercise analysis device 2, the reporting device 3, and the information display device 4B may be any combination.

### 3-2. Coordinate System

A coordinate system required in the following description is defined similarly to “1-2. Coordinate system” in the first embodiment.

### 3-3. Exercise Analysis Device

### 3-3-1. Configuration of the Exercise Analysis Device

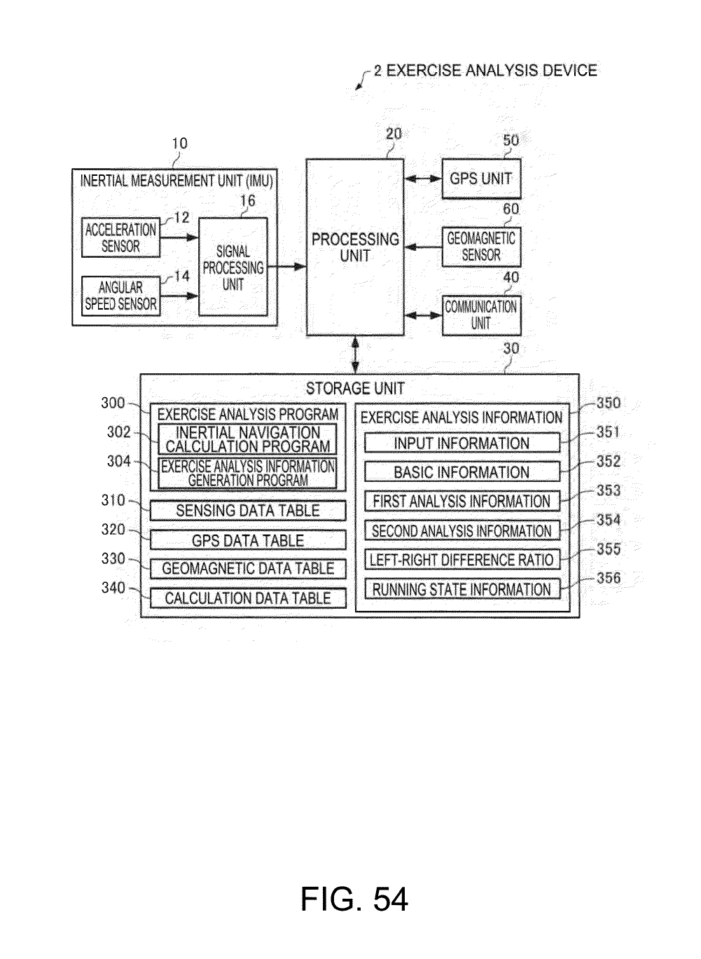

FIG. 54 is a functional block diagram illustrating an example of a configuration of an exercise analysis device 2 in the third embodiment. As illustrated in FIG. 54, the exercise analysis device 2 in the third embodiment includes an inertial measurement unit (IMU) 10, a processing unit 20, a storage unit 30, a communication unit 40, a GPS unit 50, and a geomagnetic sensor 60, similar to the first embodiment. However, in the exercise analysis device 2 of the present embodiment, some of these components may be removed or changed, or other components may be added. Since respective functions of the inertial measurement unit (IMU) 10, the GPS unit 50, and the geomagnetic sensor 60 are the same as those in the first embodiment, description thereof will be omitted.

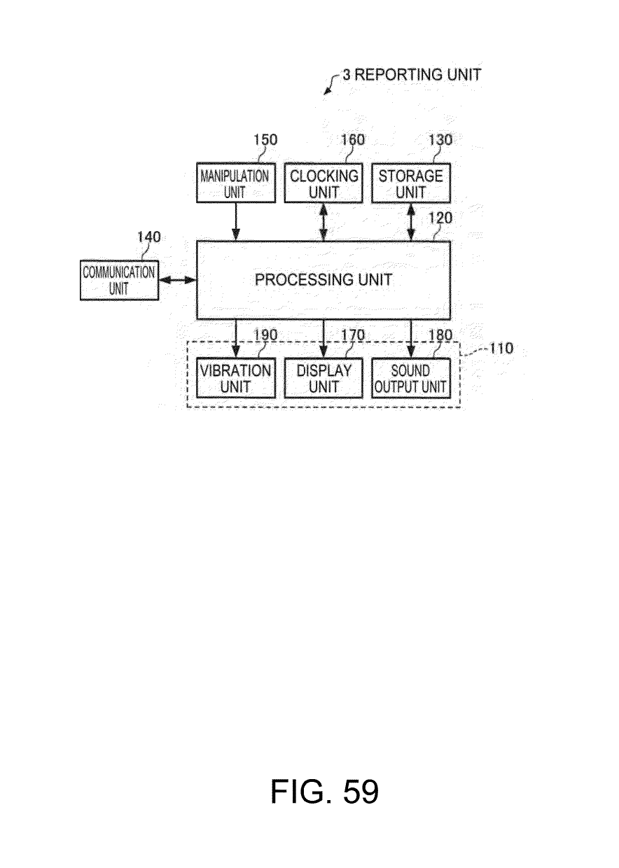

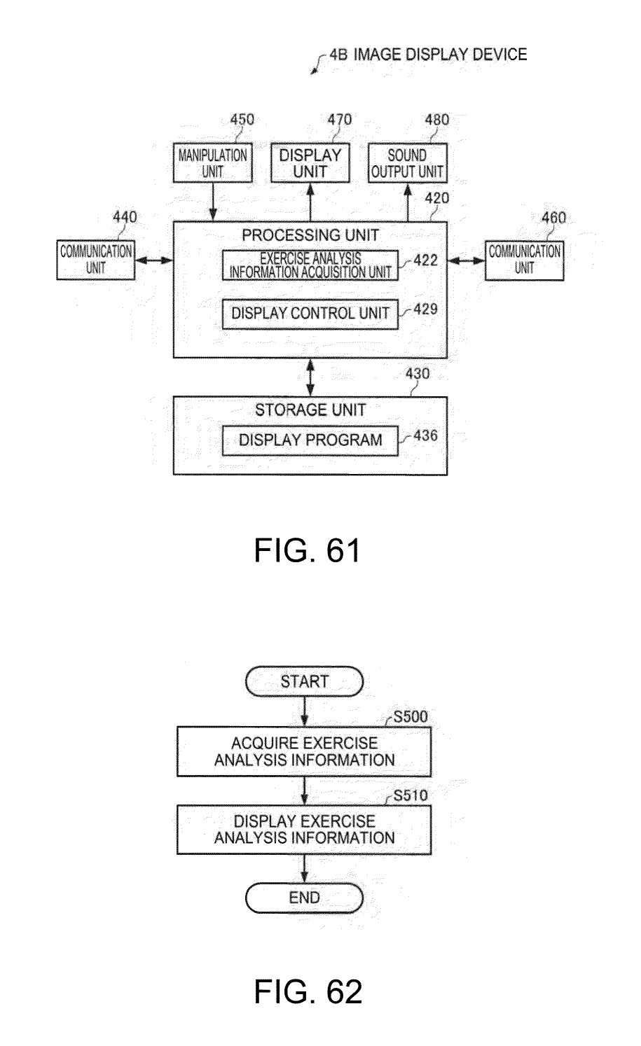

The communication unit 40 is a communication unit that performs data communication with the communication unit 140 of the reporting device 3 (see FIG. 59) or the communication unit 440 of the information display device 4B (see FIG. 61), and performs, for example, a process of receiving a command (for example, measurement start/measurement end command) transmitted from the communication unit 140 of the reporting device 3 and sending the command to the processing unit 20, a process of receiving the output information during running or the running result information generated by the processing unit 20 and transmitting the information to the communication unit 140 of the reporting device 3, or a process of receiving a transmission request command for exercise analysis information from the communication unit 440 of the information display device 4B, sending the transmission request command to the processing unit 20, receiving the exercise analysis information from the processing unit 20, and transmitting the exercise analysis information to the communication unit 440 of the information display device 4B.

The processing unit 20 includes, for example, a CPU, a DSP, and an ASIC, and performs various operation processes or control processes according to various programs stored in the storage unit 30 (recording medium), similar to the first embodiment.

Further, when the processing unit 20 receives the transmission request command for the exercise analysis information from the information display device 4B via the communication unit 40, the processing unit 20 performs a process of reading the exercise analysis information designated by the transmission request command from the storage unit 30, and sending the exercise analysis information to the communication unit 440 of the information display device 4B via the communication unit 40.

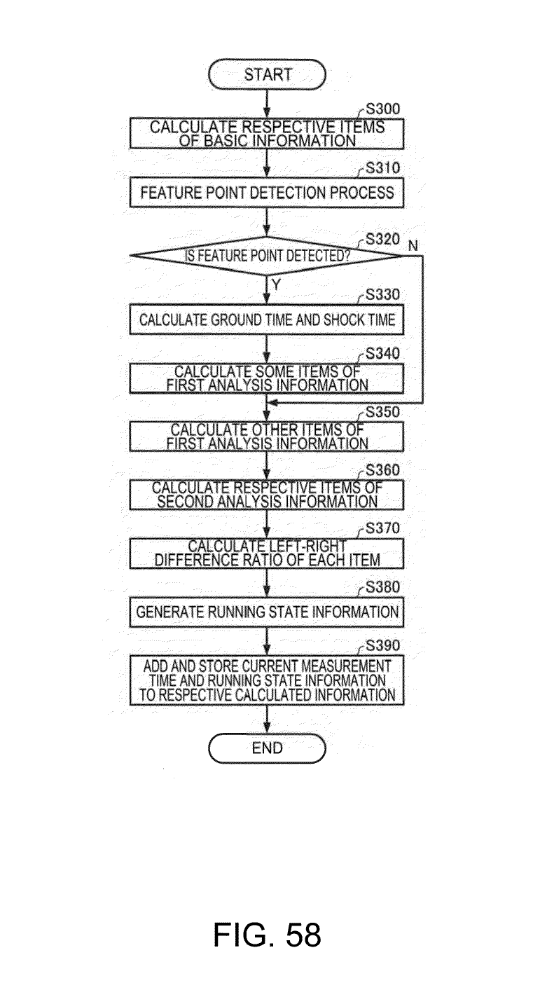

The storage unit 30 includes, for example, a recording medium that stores a program or data, such as a ROM, a flash ROM, a hard disk, or a memory card, or a RAM that is a work area of the processing unit 20. A exercise analysis program 300 read by the processing unit 20, for executing the exercise analysis process (see FIG. 14) is stored in the storage unit 30 (one of the recording media). The exercise analysis program 300 includes an inertial navigation operation program 302 for executing an inertial navigation operation process (see FIG. 15), and an exercise analysis information generation program 304 for executing the exercise analysis information generation process (see FIG. 58) as subroutines.

Further, for example, a sensing data table 310, a GPS data table 320, a geomagnetic data table 330, an operation data table 340, and exercise analysis information 350 are stored in the storage unit 30, similar to the first embodiment. Since configurations of the sensing data table 310, the GPS data table 320, the geomagnetic data table 330, and the operation data table 340 are the same as those in the first embodiment (FIGS. 4 to 7), the configurations will not be illustrated and described.

The exercise analysis information 350 is a variety of information on the exercise of the user, and includes, for example, each item of input information 351, each item of basic information 352, each item of first analysis information 353, each item of second analysis information 354, each item of a left-right difference ratio 355, and each item of running state information 356 generated by the processing unit 20.

### 3-3-2. Functional Configuration of the Processing Unit

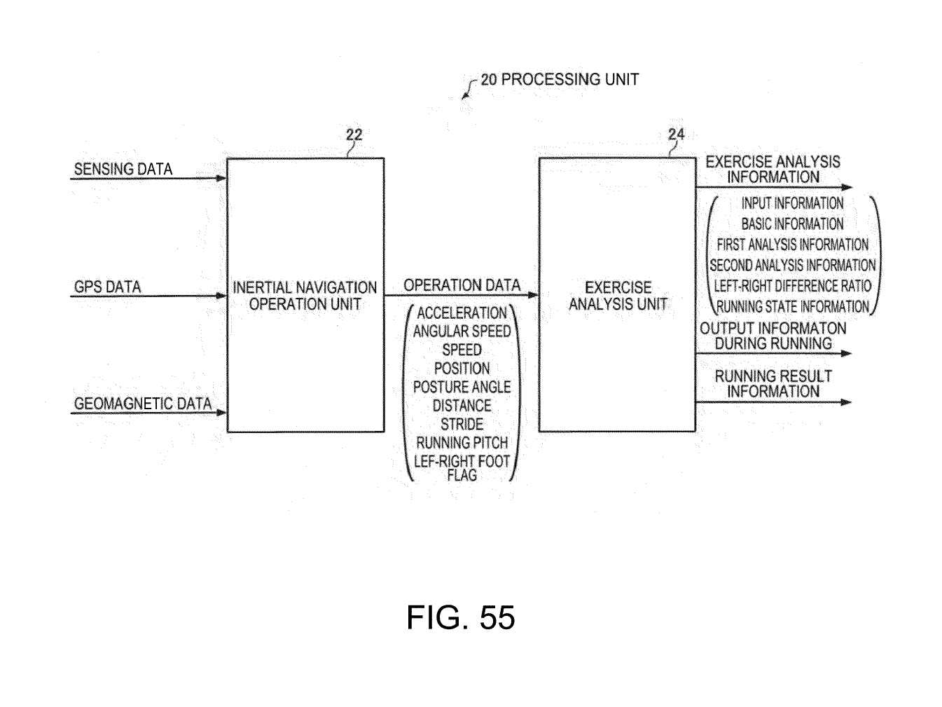

FIG. 55 is a functional block diagram illustrating an example of a configuration of the processing unit 20 of the exercise analysis device 2 in the third embodiment. In the third embodiment, the processing unit 20 executes the exercise analysis program 300 stored in the storage unit 30 to function as an inertial navigation operation unit 22 and an exercise analysis unit 24, similar to the first embodiment. However, the processing unit 20 may receive the exercise analysis program 300 stored in an arbitrary storage device (recording medium) via a network or the like and execute the exercise analysis program 300.

The inertial navigation operation unit 22 performs inertial navigation operation using the sensing data (detection result of the inertial measurement unit 10), the GPS data (detection result of the GPS unit 50), and geomagnetic data (detection result of the geomagnetic sensor 60) to calculate the acceleration, the angular speed, the speed, the position, the posture angle, the distance, the stride, and running pitch, and outputs operation data including these calculation results, similar to the first embodiment. The operation data output by the inertial navigation operation unit 22 is stored in a chronological order in the storage unit 30.

The exercise analysis unit 24 analyzes the exercise during running of the user using the operation data (operation data stored in the storage unit 30) output by the inertial navigation operation unit 22, and generates exercise analysis information (for example, input information, basic information, first analysis information, second analysis information, a left-right difference ratio, and running state information) that is information on an analysis result. The exercise analysis information generated by the exercise analysis unit 24 is stored in chronological order in the storage unit 30 during running of the user.

Further, the exercise analysis unit 24 generates output information during running that is information output during running of the user (specifically, between start and end of measurement in the inertial measurement unit 10) using the generated exercise analysis information. The output information during running generated by the exercise analysis unit 24 is transmitted to the reporting device 3 via the communication unit 40.

Further, the exercise analysis unit 24 generates the running result information that is information on the running result at the time of running end of the user (specifically, at the time of measurement end of the inertial measurement unit 10) using the exercise analysis information generated during running. The running result information generated by the exercise analysis unit 24 is transmitted to the reporting device 3 via the communication unit 40.

### 3-3-3. Functional Configuration of the Inertial Navigation Operation Unit

Since an example of a configuration of the inertial navigation operation unit 22 in the third embodiment is the same as that in the first embodiment (FIG. 9), the example is not illustrated. In the third embodiment, the inertial navigation operation unit 22 includes a bias removal unit 210, an integration processing unit 220, an error estimation unit 230, a running processing unit 240, and a coordinate transformation unit 250, similar to the first embodiment. Respective functions of these components are the same as those in the first embodiment, description thereof is omitted.

### 3-3-4. Functional Configuration of the Exercise Analysis Device

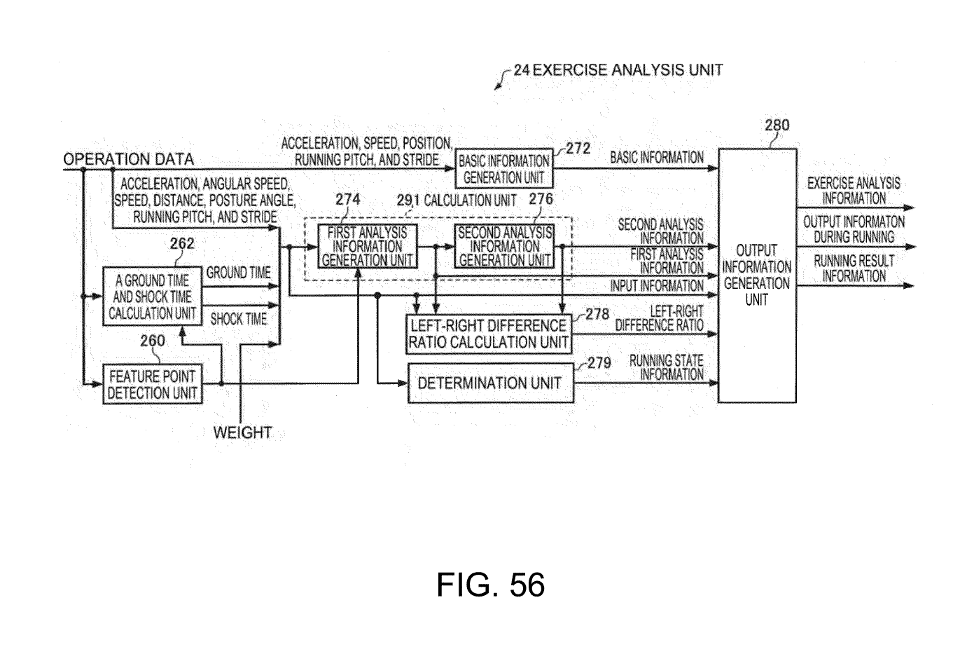

FIG. 56 is a functional block diagram illustrating an example of a configuration of the exercise analysis unit 24 in the third embodiment. In the third embodiment, the exercise analysis unit 24 includes a feature point detection unit 260, a ground time and shock time calculation unit 262, a basic information generation unit 272, a calculation unit 291, a left-right difference ratio calculation unit 278, a determination unit 279, and an output information generation unit 280. However, in the exercise analysis unit 24 of the present embodiment, some of these components may be removed or changed, or other components may be added. Since respective functions of the feature point detection unit 260, the ground time and shock time calculation unit 262, the basic information generation unit 272, and the left-right difference ratio calculation unit 278 are the same as those in the first embodiment, description thereof will be omitted.

The calculation unit 291 calculates an index regarding the running of the user using the measurement result of the inertial measurement unit 10 (an example of the detection result of the inertial sensor). In the example illustrated in FIG. 56, the calculation unit 291 includes a first analysis information generation unit 274 and a second analysis information generation unit 276. Respective functions of the first analysis information generation unit 274 and the second analysis information generation unit 276 are the same as those in the first embodiment, description thereof will be omitted.

The determination unit 279 measures a running state of the user. The running state may be at least one of the running speed and the running environment. The running environment may be, for example, a state of a slope of a running road, a state of a curve of the running road, weather, and temperature. In the present embodiment, the running speed, and the state of the slope of the running road are adopted as the running state. For example, the determination unit 279 may determine whether the running speed is “fast”, “intermediate speed”, or “slow” based on the operation data output by the inertial navigation operation unit 22. Further, for example, the determination unit 279 may determine whether the state of the slope of the running road is “ascent”, “substantially flat”, or “descent” based on the operation data output by the inertial navigation operation unit 22. The determination unit 279 may determine, for example, the state of the slope of the running road based on the data of the posture angle (pitch angle) included in the operation data. The determination unit 279 outputs the running state information that is information on the running state of the user to the output information generation unit 280.

The output information generation unit 280 performs a process of generating output information during running that is information output during running of the user using, for example, the basic information, the input information, the first analysis information, the second analysis information, the left-right difference ratio, and the running state information. Further, the output information generation unit 280 associates the above-described exercise index with the running state information to generate the output information during running.

Further, the output information generation unit 280 generates the running result information that is information of the running result of the user using, for example, the basic information, the input information, the first analysis information, the second analysis information, the left-right difference ratio, and the running state information. Further, the output information generation unit 280 associates the above-described exercise index with the running state information to generate the running result information.

Further, the output information generation unit 280 transmits the output information during running to the reporting device 3 via the communication unit 40 during running of the user, and transmits the running result information to the reporting device 3 and the information display device 4B at the time of running end of the user. Further, the output information generation unit 280 may transmit, for example, the basic information, the input information, the first analysis information, the second analysis information, the left-right difference ratio, and the running state information to the information display device 4B.

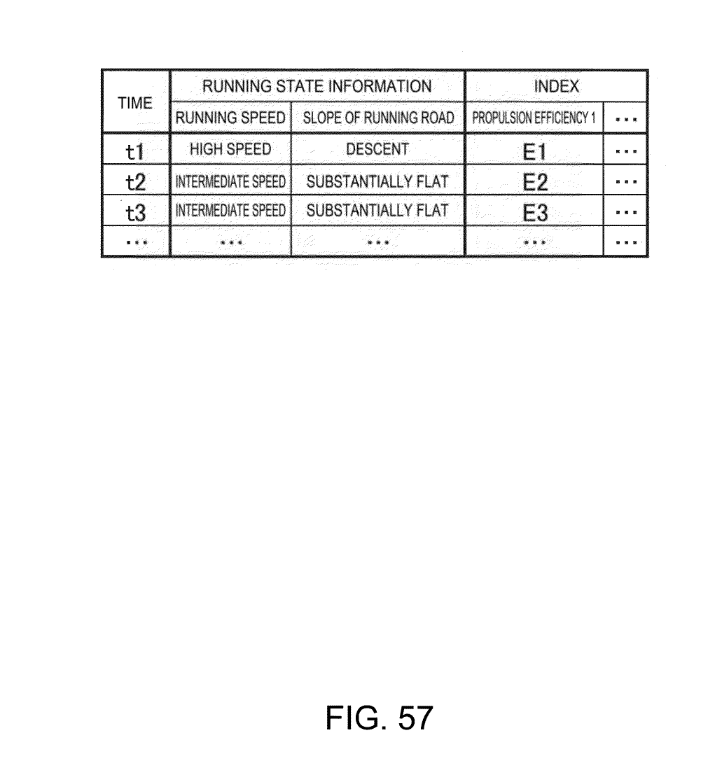

FIG. 57 is a diagram illustrating an example of a configuration of a data table of the running result information and the exercise analysis information. As illustrated in FIG. 57, in the data table of the running result information and the exercise analysis information, the time, the running state information (the running speed and the slope of running road), and the index (for example, propulsion efficiency 1) are arranged in chronological time in association with each other.

### 3-3-5. Input Information

Since each item of input information has been described in detail in “1-3-5. Input information” in the first embodiment, a description thereof will be omitted here.

### 3-3-6. First Analysis Information

Since each item of the first analysis information calculated by the first analysis information generation unit 274 has been described in detail in “1-3-6. First analysis information” in the first embodiment, a description thereof will be omitted here. Each item of the first analysis information is an item indicating the running state of the user (an example of the exercise state).

### 3-3-7. Second Analysis Information

Since each item of the second analysis information calculated by the second analysis information generation unit 276 has been described in detail in “1-3-7. Second analysis information” in the first embodiment, a description thereof will be omitted here.

### 3-3-8. Left-Right Difference Ratio (Left-Right Balance)

Since the left-right difference ratio calculated by the left-right difference ratio calculation unit 278 has been described in detail in “1-3-8. Left-right difference ratio (left-right balance)” in the first embodiment, the description thereof will be omitted here.

### 3-3-9. Procedure of the Process

Since a flowchart illustrating an example of a procedure of the exercise analysis process performed by the processing unit 20 in the third embodiment is the same as that in the first embodiment (FIG. 14), the flowchart will not be illustrated and described. Also, in the third embodiment, the exercise analysis program 300 executed by the processing unit 20 may be a portion of an information display program according to the invention. Further, a portion of the exercise analysis process corresponds to a calculation process of the information display method according to the invention (a process of calculating an index regarding running of the user using the detection result of the inertial sensor) or a determination process (process of measuring at least one of running speed and a running environment of the user).

Further, since a flowchart diagram illustrating an example of a procedure of the inertial navigation operation process (process of S40 in FIG. 14) in the third embodiment is the same as that in the first embodiment (FIG. 15), the flowchart will not be illustrated and described.

Further, since a flowchart diagram illustrating an example of a procedure of the running detection process (the process of S120 in FIG. 15) in the third embodiment is the same as that in the first embodiment (FIG. 16), the flowchart will not be illustrated and described.

FIG. 58 is a flowchart diagram illustrating an example of a procedure of an exercise analysis information generation process (the process of S50 in FIG. 14) in the third embodiment. The processing unit 20 (the exercise analysis unit 24) executes the exercise analysis information generation program 304 stored in the storage unit 30 to execute, for example, the exercise analysis information generation process in the procedure of the flowchart of FIG. 58.

An exercise analysis method illustrated in FIG. 58 includes a calculation process (S350 and S360) of calculating a measurement result of the inertial measurement unit 10, and an index regarding the running of the user.

As illustrated in FIG. 58, first, the processing unit 20 performs a process of S300 to S370, similar to the first embodiment (FIG. 17).

The processing unit 20 then generates running state information (S380).

The processing unit 20 then adds the current measurement time and the running state information to the respective information calculated in S300 to S380, stores the information in the storage unit 30 (S390), and ends the exercise analysis information generation process.

### 3-4. Reporting Device

### 3-4-1. Configuration of the Reporting Device

FIG. 59 is a functional block diagram illustrating an example of a configuration of the reporting device 3 in the second embodiment. As illustrated in FIG. 59, the reporting device 3 includes an output unit 110, a processing unit 120, a storage unit 130, a communication unit 140, a manipulation unit 150, and a clocking unit 160. However, in the reporting device 3 of the present embodiment, some of these components may be removed or changed, or other components may be added. Respective functions of the storage unit 130, the manipulation unit 150, and the clocking unit 160 are the same as those in the first embodiment, description thereof will be omitted.

The communication unit 140 is a communication unit that performs data communication with the communication unit 40 of the exercise analysis device 2 (see FIG. 54), and performs, for example, a process of receiving a command (for example, measurement start/measurement end command) according to manipulation data from the processing unit 120 and transmitting the command to the communication unit 40 of the exercise analysis device 2, or a process of receiving the output information during running or the running result information transmitted from the communication unit 40 of the exercise analysis device 2 and sending the information to the processing unit 120.

The output unit 110 outputs a variety of information sent from the processing unit 120. In the example illustrated in FIG. 59, the output unit 110 includes a display unit 170, a sound output unit 180, and a vibration unit 190. Since respective functions of the display unit 170, the sound output unit 180, and the vibration unit 190 are the same as those in the first embodiment, description thereof will be omitted.

The processing unit 120 includes, for example, a CPU, a DSP, and an ASIC, and executes a program stored in the storage unit 130 (recording medium) to perform various operation processes or control processes. For example, the processing unit 120 performs various processes according to the manipulation data received from the manipulation unit 150 (for example, a process of sending a measurement start/measurement end command to the communication unit 140, or a display process or a sound output process according to the manipulation data), a process of receiving the output information during running from the communication unit 140, generating text data or image data according to the exercise analysis information, and sending the data to the display unit 170, a process of generating sound data according to the exercise analysis information and sending the sound data to the sound output unit 180, and a process of generating vibration data according to the exercise analysis information and sending the vibration data to the vibration unit 190. Further, the processing unit 120 performs, for example, a process of generating time image data according to the time information received from the clocking unit 160 and sending the time image data to the display unit 170.

For example, when there is a worse exercise index than the reference value, the processing unit 120 reports the worse exercise index through sound or vibration, and displays the value of the worse exercise index than the reference value on the display unit 170. The processing unit 120 may generate a different type of sound or vibration according to a type of worse exercise index than the reference value, or may change the type of sound or vibration according to a degree of being worse than the reference value for each exercise index. When there are a plurality of worse exercise indexes than the reference values, the processing unit 120 may generate sound or vibration of the type according to the worst exercise index and may display information on the values of all the worse exercise indexes than the reference values, and the reference values on the display unit 170, for example, as illustrated in FIG. 19A.

The exercise index to be compared with the reference value may be all exercise indexes included in the output information during running, or may be only a specific exercise index that is determined in advance, and the user may manipulate the manipulation unit 150 or the like to select the exercise index.

The user can continue to run while recognizing which skill specification is worst and how much the skill specification is worse from a type of sound or vibration without viewing the information displayed on the display unit 170. Further, the user can accurately recognize a difference between the values of all worse exercise indexes than the reference values and the reference values when viewing the information displayed on the display unit 170.

Further, the exercise index that is a target for which sound or vibration is generated may be selected from among the exercise indexes to be compared with reference values by the user manipulating the manipulation unit 150 or the like. In this case, for example, information on the values of all the worse exercise indexes than the reference values, and the reference values may be displayed on the display unit 170.

Further, the user may perform setup of a reporting period (for example, setup such as generation of sound or vibration for 5 seconds every one minute) through the manipulation unit 150, and the processing unit 120 may perform reporting to the user according to the set reporting period.

Further, in the present embodiment, the processing unit 120 acquires the running result information transmitted from the exercise analysis device 2 via the communication unit 140, and displays the running result information on the display unit 170. For example, as illustrated in FIG. 19B, the processing unit 120 displays an average value of each exercise index during running of the user, which is included in the running result information, on the display unit 170. When the user views the display unit 170 after the running end (after the measurement end manipulation), the user can immediately recognize the goodness or badness of each exercise index.

### 3-4-2. Procedure of the Process

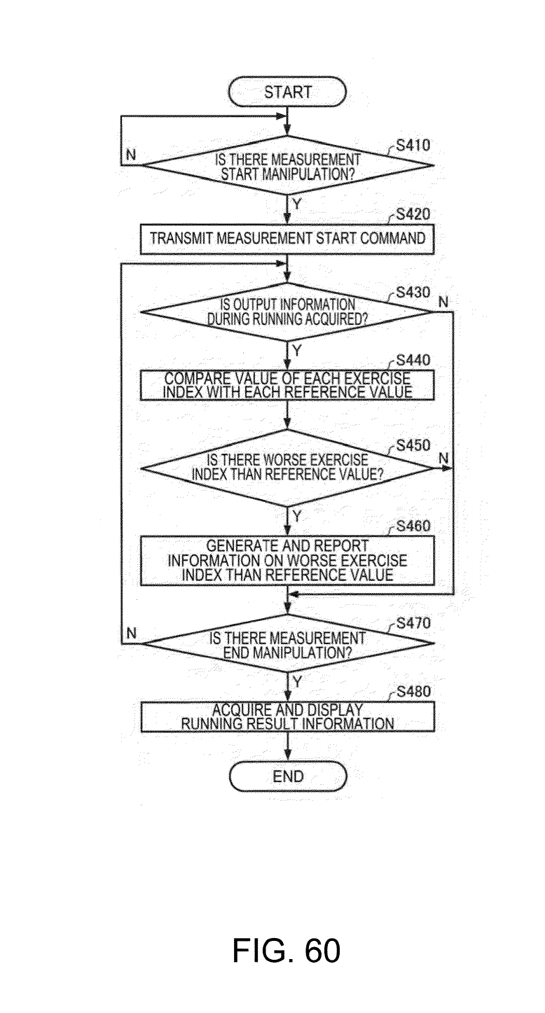

FIG. 60 is a flowchart diagram illustrating an example of a procedure of a reporting process performed by the processing unit 120 in the third embodiment. The processing unit 120 executes the program stored in the storage unit 130, for example, to execute the reporting process in the procedure of the flowchart of FIG. 60.

As illustrated in FIG. 60, the processing unit 120 first waits until the processing unit 120 acquires the manipulation data of measurement start from the manipulation unit 150 (N in S410). When the processing unit 120 acquires the manipulation data of measurement start (Y in S410), the processing unit 120 transmits the measurement start command to the exercise analysis device 2 via the communication unit 140 (S420).

Then, the processing unit 120 compares the value of each exercise index included in the acquired output information during running with each reference value acquired in S400 (S440) each time the processing unit 120 acquires the output information during running from the exercise analysis device 2 via the communication unit 140 (Y in S430) until the processing unit 120 acquires the manipulation data of the measurement end from the manipulation unit 150 (N in S470).

When there is a worse exercise index than the reference value (Y in S450), the processing unit 120 generates information on the worse exercise index than the target value and reports the information to the user using sound, vibration, text, or the like via the sound output unit 180, the vibration unit 190, and the display unit 170 (S460).

On the other hand, when there is no worse exercise index than the reference value (N in S450), the processing unit 120 does not perform the process of S460.

Also, when the processing unit 120 acquires the manipulation data of the measurement end from the manipulation unit 150 (Y in S470), the processing unit 120 acquires the running result information from the exercise analysis device 2 via the communication unit 140, displays the running result information on the display unit 170 (S480), and ends the reporting process.

Thus, the user can run while recognizing the running state based on the information reported in S450. Further, the user can immediately recognize the running result after running end, based on the information displayed in S480.

### 3-5-1. Configuration of the Information Display Device

FIG. 61 is a functional block diagram illustrating an example of a configuration of the information display device 4B. As illustrated in FIG. 61, the information display device 4B includes a processing unit 420, a storage unit 430, a communication unit 440, a manipulation unit 450, a communication unit 460, a display unit 470, and a sound output unit 480, similar to the exercise analysis device 2 in the first embodiment. However, in the information display device 4B of the present embodiment, some of these components may be removed or changed, or other components may be added.

The communication unit 440 is a communication unit that performs data communication with the communication unit 40 of the exercise analysis device 2 (see FIG. 54) or the communication unit 140 of the reporting device 3 (see FIG. 140). The communication unit 440 performs, for example, a process of receiving the transmission request command for requesting transmission of the exercise analysis information designated according to the manipulation data (exercise analysis information included in the running data that is a registration target) from the processing unit 420, transmitting the transmission request command to the communication unit 40 of the exercise analysis device 2, receiving the exercise analysis information from the communication unit 40 of the exercise analysis device 2, and sending the exercise analysis information to the processing unit 420.

The communication unit 460 is a communication unit that performs data communication with the server 5, and performs, for example, a process of receiving running data that is a registration target from the processing unit 420 and transmitting the running data to the server 5 (running data registration process), and a process of receiving management information corresponding to manipulation data of editing, deletion, and replacement of the running data from the processing unit 420 and transmitting the management information to the server 5.

The manipulation unit 450 performs a process of acquiring manipulation data from the user (for example, manipulation data of registration, editing, deletion, replacement of the running data), and sending the manipulation data to processing unit 420. The manipulation unit 450 may be, for example, a touch panel display, a button, a key, or a microphone.

The display unit 470 displays image data or text data sent from the processing unit 420 as a text, a graph, a table, animation, or other images. The display unit 470 is implemented by, for example, a display such as an LCD, an organic EL display, or an EPD, and may be a touch panel display. Also, functions of the manipulation unit 450 and the display unit 470 may be implemented by one touch panel display. The display unit 470 in the present embodiment displays the running state information that is information on the running state of the user (at least one of the running speed and the running environment of the user) and the index regarding the running of the user in association with each other.

The sound output unit 480 outputs sound data sent from the processing unit 420 as sound such as voice or buzzer sound. The sound output unit 480 is implemented by, for example, a speaker or a buzzer.

The storage unit 430 includes, for example, a recording medium that stores a program or data, such as a ROM, a flash ROM, a hard disk, or a memory card, or a RAM that is a work area of the processing unit 420. A display program 436 read by the processing unit 420, for executing the display process (see FIG. 62) is stored in the storage unit 430 (one of the recording media).

The processing unit 420 includes, for example, a CPU, a DSP, and an ASIC, and executes various programs stored in the storage unit 430 (recording medium) to perform various operation processes or control processes. For example, the processing unit 420 performs a process of transmitting a transmission request command for requesting transmission of the exercise analysis information designated according to the manipulation data received from the manipulation unit 450 to the exercise analysis device 2 via the communication unit 440, and receiving the exercise analysis information from the exercise analysis device 2 via the communication unit 440, or a process of generating running data including the exercise analysis information received from the exercise analysis device 2 according to the manipulation data received from the manipulation unit 450, and transmitting the running data to the server 5 via the communication unit 460. Further, the processing unit 420 performs a process of transmitting management information according to the manipulation data received from the manipulation unit 450 to the server 5 via the communication unit 460.

In particular, in the present embodiment, the processing unit 420 executes the display program 436 stored in the storage unit 430 to function as an exercise analysis information acquisition unit 422 and a display control unit 429. However, the processing unit 420 may receive and execute the display program 436 stored in any storage device (recording medium) via a network or the like.

The exercise analysis information acquisition unit 422 performs a process of acquiring exercise analysis information that is information on the analysis result of the exercise of the user who is an analysis target from the database of the server 5 (or the exercise analysis device 2). The exercise analysis information acquired by the exercise analysis information acquisition unit 422 is stored in the storage unit 430. This exercise analysis information may be generated by the same exercise analysis device 2 or may be generated by any one of a plurality of different exercise analysis devices 2. The plurality of pieces of exercise analysis information acquired by the exercise analysis information acquisition unit 422 include various exercise indexes of the user (for example, various exercise indexes described above) and the running state information are association with each other.

The display control unit 429 performs a display process of controlling the display unit 470 based on the exercise analysis information acquired by the exercise analysis information acquisition unit 422.

### 3-5-2. Procedure of the Process

FIG. 62 is a flowchart diagram illustrating an example of a procedure of a display process performed by the processing unit 420. The processing unit 420 executes the display program 436 stored in the storage unit 430, for example, to execute a display process in the procedure of the flowchart of FIG. 62. The display program 436 may be a portion of the information display program according to the invention. Further, a portion of the display process corresponds to a display process of the information display method according to the invention (in which the running state information that is information on the running state of the user (at least one of the running speed and the running environment), and the index regarding running of the user are displaced in association with each other).

First, the processing unit 420 acquires the exercise analysis information (S500). In the present embodiment, the exercise analysis information acquisition unit 422 of the processing unit 420 acquires the exercise analysis information via the communication unit 440.

Then, the processing unit 420 displays the exercise analysis information (S510). In the present embodiment, the display control unit 429 of the processing unit 420 displays the exercise analysis information based on the exercise capability information acquired by the exercise analysis information acquisition unit 422 of the processing unit 420.

Through the process, the display unit 470 displays the running state information that is information on the running state of the user (at least one of the running speed and the running environment), and the index regarding running of the user in association with each other.

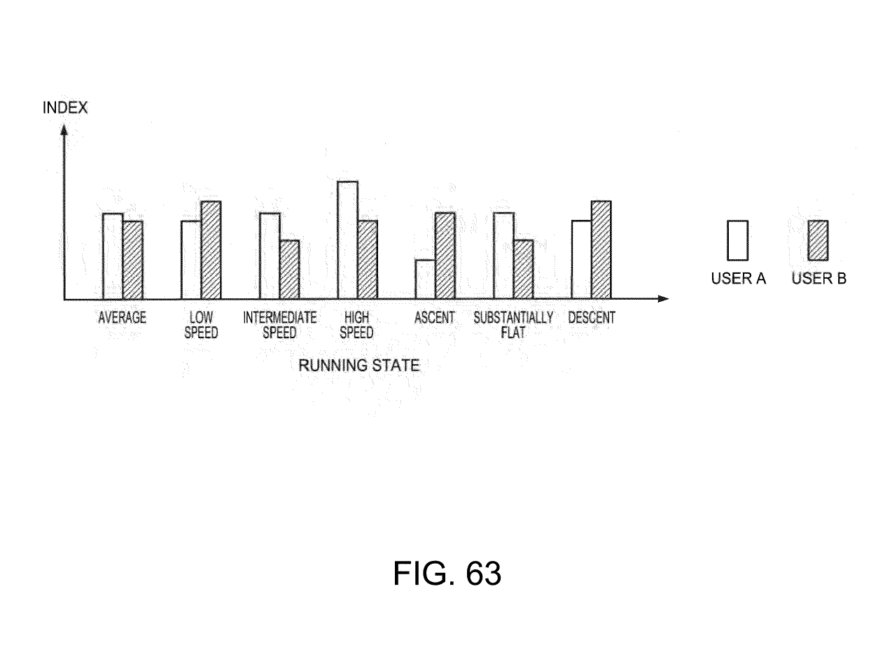

FIG. 63 is a diagram illustrating an example of exercise analysis information displayed on the display unit 470. In the example of FIG. 63, the exercise analysis information displayed on the display unit 470 includes a bar graph in which one exercise index (for example, the above-described propulsion efficiency 1) in running of a period of the analysis target of two users (user A and user B) is relatively evaluated. A horizontal axis in FIG. 63 indicates a running state, and a vertical axis indicates a relative evaluation value of the index.

In the example of FIG. 63, a good running state or a weak running state of each user can be seen. For example, it can be seen that user A is weak when the running state is an ascent. Thus, it can be seen that for user A, a total running time is highly likely to be shortened by intensively improving the index of the ascent. Accordingly, efficient training is possible.

### 3-6. Effects

According to the third embodiment, since the running state information and the index are displayed in association with each other, indexes of different forms primarily caused by a difference in the running state can be divided and displayed. Therefore, it is possible to implement the information system 1B capable of accurately recognizing indexes regarding the running of the user.

Further, according to the third embodiment, since the determination unit 279 determines the running state, it is possible to implement the information display system 1B capable of reducing input manipulations of the user.

Further, according to the third embodiment, the indexes of different forms primarily caused by a difference in the running state can be divided and displayed by adopting the running speed or a state of a slope of a running road that easily affects the form, as a running state. Therefore, it is possible to implement an information display system 1B capable of accurately recognizing indexes regarding the running of the user.

### 4. Modification Example

The invention is not limited to the present embodiment, and various modifications can be made within the scope of the invention. Hereinafter, a modification example will be described. Also, the same components as those in the above embodiment are denoted with the same reference numerals, and repeated description will be omitted.

### 4-1. Sensor

While the acceleration sensor 12 and the angular speed sensor 14 are integrally formed as the inertial measurement unit 10 and embedded in the exercise analysis device 2 in each embodiment, the acceleration sensor 12 and the angular speed sensor 14 may not be integrally formed. Alternatively, the acceleration sensor 12 and the angular speed sensor 14 may be directly mounted on the user instead of being embedded in the exercise analysis device 2. In either case, for example, one of sensor coordinate systems may be the b frame in the embodiment, the other sensor coordinate system may be converted into the b frame, and the embodiment may be applied.

Further, while a portion of the user on which the sensor (exercise analysis device 2 (IMU10)) is amounted has been described as the waist in each embodiment, the sensor may be mounted on a portion other than the waist. However, a suitable amounting portion is a trunk (a portion other than a limb) of the user. However, the amounting portion is not limited to the trunk, and the sensor may be mounted on, for example, a head or a foot other than the arm. Further, the number of sensors is not limited to one, and an additional sensor may be mounted on another portion of the body. For example, sensors may be mounted on a waist and a leg or a waist and an arm.

### 4-2. Inertial Navigation Operation

While the integration processing unit 220 calculates speed, a position, a posture angle, and a distance of the e frame, and the coordinate transformation unit 250 coordinate-transforms the speed, the position, the posture angle, and the distance of the e frame into speed, a position, a posture angle, and a distance of the m frame in each embodiment, the integration processing unit 220 may calculate the speed, the position, the posture angle, and the distance of the m frame. In this case, since the exercise analysis unit 24 may perform the exercise analysis process using the speed, the position, the posture angle, and the distance of the m frame calculated by the integration processing unit 220, the coordinate transformation of the speed, the position, the posture angle, and the distance in the coordinate transformation unit 250 is unnecessary. Further, the error estimation unit 230 may perform error estimation using an extended Kalman filter, using the speed, the position, and the posture angle of the m frame.

Further, while the inertial navigation operation unit 22 performs a portion of the inertial navigation operation using a signal from a GPS satellite in each embodiment, a signal from a position measurement satellite of a global navigation satellite system (GNSS) other than the GPS or a position measurement satellite other than the GNSS may be used. For example, one or two or more of satellite position measurement systems: a wide area augmentation system (WAAS), a quasi zenith satellite system (QZSS), a GLObal NAvigation Satellite System (GLONASS), a GALILEO, and a BeiDou Navigation Satellite System (BeiDou) may be used. Further, an indoor messaging system (IMES) or the like may be used.

Further, the running detection unit 242 detects the running period at a timing at which the acceleration of the vertical movement of the user (z-axis acceleration) is equal to or greater than a threshold value and becomes a maximum value in each embodiment, but the invention is not limited thereto. For example, the running detection unit 242 may detect the running period at a timing at which the acceleration of the vertical movement of the user (z-axis acceleration) is changed from positive to negative (or a timing at which the acceleration is changed from negative to positive). Alternatively, the running detection unit 242 may integrate acceleration of a vertical movement (z-axis acceleration) to calculate speed of the vertical movement (z-axis speed), and detect the running period using the speed of the vertical movement (z-axis speed). In this case, for example, the running detection unit 242 may detect the running period at a timing at which the speed crosses a threshold value near a center value of a maximum value and a minimum value according to an increase in the value or according to a decrease in the value. Further, for example, the running detection unit 242 may calculate a resultant acceleration of the x axis, the y axis, and the z axis and detect the running period using the calculated resultant acceleration. In this case, for example, the running detection unit 242 may detect the running period at a timing at which the resultant acceleration crosses a threshold value near a center value of a maximum value and a minimum value according to an increase in the value or according to a decrease in the value.

Further, while the error estimation unit 230 uses the speed, the posture angle, the acceleration, the angular speed, and the position as state variables, and estimates an error thereof using the extended Kalman filter in each embodiment, the error estimation unit 230 may use some of the speed, the posture angle, the acceleration, the angular speed, and the position as the state variables, and estimate an error thereof. Alternatively, the error estimation unit 230 may use something (for example, movement distance) other than the speed, the posture angle, the acceleration, the angular speed, and the position as state variables, and estimate an error thereof.

Further, while the extended Kalman filter is used for the error estimation unit 230 to estimate the error in each embodiment, such a filter may be replaced with another estimation means, such as a particle filter or an Hoc (H Infinity) filer.

### 4-3. Exercise Analysis Process

While in each embodiment, the exercise analysis device 2 performs the process of generating the exercise analysis information (exercise index), the exercise analysis device 2 may transmit measurement data of the inertial measurement unit 10 or the operation result (operation data) of the inertial navigation operation to the server 5, and the server 5 may perform the process of generating the exercise analysis information (exercise index) (function as the exercise analysis device) using the measurement data or the operation data, and store the exercise analysis information in the database.

Further, for example, the exercise analysis device 2 may generate the exercise analysis information (exercise index) using the biometric information of the user. For example, skin temperature, central portion temperature, an amount of oxygen consumption, a change in pulsation, a heart rate, a pulse rate, a respiratory rate, skin temperature, a central portion body temperature, a heat flow, a galvanic skin response, an electromyogram (EMG), an electroencephalogram (EEG), an electrooculogram (EOG), blood pressure, an amount of oxygen consumption, activity, a change in pulsation, or a galvanic skin response is considered as the biological information. The exercise analysis device 2 may include a device that measures biological information, and the exercise analysis device 2 may receive biological information measured by the measuring device. For example, the user may wear a wristwatch type pulse meter or a heart rate sensor wound from a belt to a chest, and run, and the exercise analysis device 2 may calculate the heart rate during running of the user using a measurement value of the pulse meter or the heart rate sensor.

While the exercise indexes included in the exercise analysis information are indexes regarding skill power of users in each embodiment, the exercise analysis information may include exercise indexes regarding endurance power. For example, the exercise analysis information may include a heart rate reserved (HRR) calculated as (heart rate−heart rate at rest)/(maximum heart rate−heart rate at rest)×100 as the exercise index regarding endurance. For example, each player may operate the reporting device 3 to input the heart rate, the maximum heart rate, and the heart rate at rest each time the player runs, or the player may wear a heart rate meter and run, and the exercise analysis device 2 may acquire values of the heart rate, the maximum heart rate, and the heart rate at rest from the reporting device 3 or the heart rate meter, and calculate a value of the heart rate reserve (HRR).

While the exercise analysis in running of a person is a target in each embodiment, the invention is not limited thereto and can be similarly applied to exercise analysis in walking or running of a moving body, such as an animal or a walking robot. Further, the invention is not limited to the running, and can be applied to a wide variety of exercises such as ascent, trail running, skiing (including cross-country and ski jumping), snowboarding, swimming, running of a bicycle, skating, golf, tennis, baseball, and rehabilitation. When the invention is applied to the skiing, for example, a determination may be performed as to whether carving is clearly generated or the ski plate is shifted from a difference in acceleration in the vertical direction when a ski plate is pressed, or the right foot and left foot or sliding capability may be determined from a locus of a change the acceleration in the vertical direction when a ski plate is pressed and unweighted. Alternatively, analysis may be performed as to how much a locus of a change in the angular speed in the yaw direction is close to a sine wave to determine whether the user skies, or analysis may be performed as to how much a locus of a change in the angular speed in the roll direction is close to a sine wave to determine whether smooth sliding is possible.

### 4-4. Reporting Process

While in each embodiment, the reporting device 3 reports the exercise index worse than the target value or the reference value to the user through sound or vibration when there is such an exercise index, the reporting device 3 may report an exercise index better than the target value or the reference value to the user through sound or vibration when there is such an exercise index.

Further, while the reporting device 3 performs the comparison process between the value of each exercise index and the target value or the reference value in each embodiment, the exercise analysis device 2 may perform this comparison process and control output or display of the sound or vibration of the reporting device 3 according to a comparison result.

Further, while the reporting device 3 may be a wristwatch type device in each embodiment, the invention is not limited thereto, and the reporting device 3 may be a non-wristwatch type portable device mounted on a user (head-mounted display (HMD), a device mounted on a waist of a user (which may be the exercise analysis device 2), or a non-mounting type portable device (for example, a smart phone). When the reporting device 3 is the head-mounted display (HMD), the display unit is sufficiently larger and has higher visibility than a display unit of a wristwatch type reporting device 3, and thus, the user viewing the display unit does not obstruct the running. Accordingly, for example, information on a running transition of the user up to the current time (information as illustrated in FIG. 29) may be displayed, or an image in which a virtual runner created based on a time (for example, a time set by the user, a record of the user, a record of a famous person, or a world record) runs may be displayed.

### 4-5. Others

While in the first embodiment, the information analysis device 4 performs the analysis process, the server 5 may perform the analysis process (function as the information analysis device) and the display server 5 may transmit the analysis information to the display device over the network.

Further, while in the second embodiment, the image generation device 4A performs the image generation process, the server 5 may perform the image generating process (function as the image generation device) and the server 5 may transmit the image information to the display device over the network. Alternatively, the exercise analysis device 2 may perform the image generation process (function as the image generation device) and transmit image information to the reporting device 3 or any display device. Alternatively, the reporting device 3 may perform the image generation process (function as the image generation device) and display the generated image information on the display unit 170. The exercise analysis device 2 or the reporting device 3 functioning as the image generation device 4A or the image generation device may perform image processing after running of the user ends (after the measurement ends). Alternatively, the exercise analysis device 2 or the reporting device 3 functioning as the image generation device 4A or the image generation device may perform the image generation process in the running of the user, and the generated image may be displayed in real time during running of the user.

Further, in the second embodiment described above, the processing unit 420 (image information generation unit 428) of the image generation device 4A generates the image data in each step and updates displaying, but the invention is not limited thereto. For example, the processing unit 420 may calculate the average value of each exercise index for each feature points at arbitrary intervals (for example, 10 minutes), and generate the image data using the average value of each exercise index of a calculation result. Alternatively, the processing unit 420 (image information generation unit 428) of the image generation device 4A may calculate an average value of each exercise index for each feature point from start of running of the user to end (from measurement start to measurement end), and generate each pieces of image data using the average value of each exercise index of a calculation result.

Further, while in the second embodiment, the processing unit 420 (image information generation unit 428) of the image generation device 4A calculates the value of the dropping of the waist that is an exercise index using the value of the distance in the vertical direction included in the exercise analysis information when generating the image data of the mid-stance, the processing unit 20 (the exercise analysis unit 24) of the exercise analysis device 2 may generate exercise analysis information also including the value of dropping of the waist as an exercise index.

Further, while in the second embodiment, the processing unit 420 (image information generation unit 428) of the image generation device 4A detects the feature point of the exercise of the user using the exercise analysis information, the processing unit 20 of the exercise analysis device 2 may detect the feature point necessary for the image generation process, and generate the exercise analysis information including information on the detected feature point. For example, the processing unit 20 of the exercise analysis device 2 may add a detection flag different for each type of feature point to data of a time at which the feature point is detected, to generate exercise analysis information including information on the feature point. Also, the processing unit 420 (image information generation unit 428) of the image generation device 4A may perform the image generation process using the information on the feature point included in the exercise information.

Further, while the running data (exercise analysis information) of the user is stored in the database of the server 5 in each embodiment, the running data may be stored in a database built in the storage unit 430 of the information analysis device 4, the image generation device 4A, or the information display device 4B. That is, the server 5 may be removed.

For example, the exercise analysis device 2 or the reporting device 3 may calculate a score of the user from the input information or the analysis information, and report the score during running or after running. For example, the numerical value of each exercise index may be divided into a plurality of steps (for example, 5 steps or 10 steps), and the score may be determined for each step. Further, for example, the exercise analysis device 2 or the reporting device 3 may assign a score according to a type or the number of the exercise index of a good record, or the total score may be calculated and displayed.

Further, while the GPS unit 50 is provided in the exercise analysis device 2 in each embodiment, the GPS unit 50 may be provided in the reporting device 3. In this case, the processing unit 120 of the reporting device 3 may receive GPS data from the GPS unit 50, and transmit the GPS data to the exercise analysis device 2 via the communication unit 140, and the processing unit 20 of the exercise analysis device 2 may receive the GPS data via the communication unit 40, and add the received GPS data to the GPS data table 320.

Further, while the exercise analysis device 2 and the reporting device 3 are separate bodies in each embodiment, the exercise analysis device 2 and the reporting device 3 may integrated for an exercise analysis device.

Further, while in the third embodiment described above, the exercise analysis device 2 and the information display device 4B are separate bodies, the exercise analysis device 2 and the information display device 4B may be integrated for and information display device.

Further, while in the each embodiment described above, the exercise analysis device 2 is mounted on the user, the invention is not limited thereto, and the inertial measurement unit (inertial sensor) or the GPS unit may be mounted in, for example, the torso of the user, the inertial measurement unit (inertial sensor) or the GPS unit may transmit a detection result to a portable information device such as a smart phone, an installation type of information device such as a personal computer, or a server over a network, and such a device may analyze the exercise of the user using the received detection result. Alternatively, an inertial measurement unit (inertial sensor) or the GPS unit mounted on, for example, the torso of the user may record the detection result in a recording medium such as a memory card, and the information device such as a smart phone or a personal computer may read the detection result from the recording medium and perform the exercise analysis process.

Each embodiment and each modification example described above are examples, and the invention is not limited thereto. For example, each embodiment and each modification example can be appropriately combined.

The invention includes substantially the same configuration (for example, a configuration having the same function, method, and result or a configuration having the same purpose and effects) as the configuration described in the embodiment. Further, the invention includes a configuration in which a non-essential portion in the configuration described in the embodiment is replaced. Further, the invention includes a configuration having the same effects as the configuration described in the embodiment or a configuration that can achieve the same purpose. Further, the invention includes a configuration in which a known technology is added to the configuration described in the embodiment.

The entire disclosure of Japanese Patent Application No. 2014-157206, filed Jul. 31, 2014 and No. 2014-157209, filed Jul. 31, 2014 and No. 2014-157210, filed Jul. 31, 2014 and No. 2015-115212, filed Jun. 5, 2015 are expressly incorporated by reference herein.

What is claimed is:

1. An information analysis device comprising:
an exercise analysis information acquisition unit that acquires a plurality of pieces of exercise analysis information that are results of analyzing exercise of a plurality of users; and
an analysis information generation unit that generates analysis information from which exercise capabilities of the plurality of users can be compared, using the plurality of pieces of exercise analysis information.

2. The information analysis device according to claim 1,
wherein the analysis information generation unit generates the analysis information from which exercise capabilities of the plurality of users are comparable each time the plurality of users perform the exercise.

3. The information analysis device according to claim 1,
wherein the plurality of users are classified into a plurality of groups, and
the analysis information generation unit generates the analysis information from which exercise capabilities of the plurality of users are comparable for each group.

4. The information analysis device according to claim 1,
wherein each of the plurality of pieces of exercise analysis information includes a value of an index regarding exercise capability of each of the plurality of users, and
the analysis information generation unit generates the analysis information from which exercise capability of the first user included in the plurality of users is relatively evaluable, using the values of the indexes of the plurality of users.

5. The information analysis device according to claim 1,
wherein each of the plurality of pieces of exercise analysis information includes a value of an index regarding exercise capability of each the plurality of users,
the information analysis device includes a target value acquisition unit that acquires a target value of the index of the first user included in the plurality of users, and
the analysis information generation unit generates the analysis information from which the value of the index of the first user is comparable with the target value.

6. The exercise analysis device according to claim 4,
wherein the index is at least one of ground time, stride, energy, a directly-under landing rate, propulsion efficiency, a flow of a leg, an amount of brake at the time of landing, and landing shock.

7. The exercise analysis device according to 1,
wherein the exercise capability is skill power or endurance power.

8. An exercise analysis system, comprising:
an exercise analysis device that analyzes exercise of a user using a detection result of an inertial sensor and generates exercise analysis information that is information on an analysis result; and
the information analysis device according to claim 5.

9. The exercise analysis system according to claim 8, comprising:
a reporting device that reports information on an exercise state during exercise of a first user included in the plurality of users,
the information analysis device transmits the target value to the reporting device,
the exercise analysis device transmits a value of the index to the reporting device during exercise of the first user, and
the reporting device receives the target value and the value of the index, compares the value of the index with the target value, and reports information on the exercise state according to a comparison result.

10. The movement analysis system according to claim 9,
wherein the reporting device reports information on the exercise state through sound or vibration.

11. An information analysis method, comprising:
acquiring a plurality of pieces of exercise analysis information that is a result of analyzing exercises of a plurality of users using a detection result of an inertial sensor; and
generating analysis information from which exercise capabilities of the plurality of users can be compared, using the plurality of pieces of exercise analysis information.

12. An analysis program causing a computer to execute:
acquisition of a plurality of pieces of exercise analysis information that is a result of analyzing exercises of a plurality of users using a detection result of an inertial sensor; and
generation of analysis information from which exercise capabilities of the plurality of users can be compared, using the plurality of pieces of exercise analysis information.

13. An image generation device, comprising:
an exercise analysis information acquisition unit that acquires exercise analysis information of a user at the time of running, the exercise analysis information being generated using a detection result of an inertial sensor; and
an image information generation unit that generates image information in which the exercise analysis information is associated with image data of a user object indicating running of the user.

14. The image generation device according to claim 13,
wherein the exercise analysis information includes a value of at least one index regarding exercise capability of the user.

15. The image generation device according to claim 13,
wherein the image information generation unit calculates a value of at least one index regarding exercise capability of the user using the exercise analysis information.

16. The image generation device according to claim 14,
wherein the exercise analysis information includes information on the posture angle of the user, and
the image information generation unit generates the image information using the value of the index and the information on the posture angle.

17. The image generation device according to claim 13,
wherein the image information generation unit generates comparison image data for comparison with the image data, and generates the image information including the image data and the comparison image data.

18. The image generation device according to claim 13,
wherein the image data is image data indicating an exercise state at a feature point of the exercise of the user.

19. The image generation device according to claim 18,
wherein the feature point is a time at when a foot of the user lands, a time of mid-stance, or a time of kicking.

20. The image generation device according to claim 13,
wherein the image information generation unit generates the image information including a plurality of pieces of image data respectively indicating exercise states at multiple types of feature points of the exercise of the user.

21. The image generation device according to claim 20,
wherein at least one of multiple types of feature points is a time at when a foot of the user lands, a time of mid-stance, or a time of kicking.

22. The image generation device according to claim 20,
wherein, in the image information, the plurality of pieces of image data are arranged side by side on a time axis or a space axis.

23. The image generation device according to claim 22,
wherein the image information generation unit generates a plurality of pieces of supplement image data for supplementing the plurality of pieces of image data on a time axis or on a spatial axis, and generates the image information including moving image data having the plurality of pieces of image data and the plurality of pieces of supplement image data.

24. The image generation device according to claim 13,
wherein the inertial sensor is mounted to a torso of the user.

25. An exercise analysis system, comprising:
the image generation device according to claim 13; and
an exercise analysis device that generates the exercise analysis information.

26. An image generation method, comprising:
acquiring exercise analysis information of a user at the time of running, the exercise analysis information being generated using a detection result of an inertial sensor; and
generating image information in which the exercise analysis information is associated with image data of a user object indicating running of the user.

27. An image generation program causing a computer to execute:
acquisition of exercise analysis information of a user at the time of running, the exercise analysis information being generated using a detection result of an inertial sensor; and
generation of image information in which the exercise analysis information is associated with image data of a user object indicating running of the user.

28. An information display device, comprising:
a display unit that displays running state information that is information on at least one of running speed and a running environment of a user, and an index regarding running of the user calculated using a detection result of an inertial sensors in association with each other.

29. The information display device according to claim 28,
wherein the running environment is a state of a slope of a running road.

30. The information display device according to claim 28,
wherein the index is any one of directly-under landing, propulsion efficiency, a flow of a leg, a running pitch, and landing shock.

31. An information display system, comprising:
a calculation unit that calculates an index regarding running of a user using a detection result of an inertial sensor; and
a display unit that displays running state information that is information on at least one of running speed and a running environment of the user, and the index in association with each other.

32. The information display system according to claim 31, further comprising:
a determination unit that measures at least one of the running speed and the running environment.

33. An information display program causing a computer to execute:
displaying of running state information that is information on at least one of running speed and a running environment of a user, and an index regarding running of the user calculated using a detection result of an inertial sensors in association with each other.

34. An information display method, comprising:
displaying running state information that is information on at least one of running speed and a running environment of a user, and an index regarding running of the user calculated using a detection result of an inertial sensors in association with each other.
